SEQUENCE LISTING (48 sequences)

SEQ ID NO: 1 - DNA, 729 bp, Macaca fascicularis
gagaagtattggtgtaagtggagtaacacaggctgccagaccctgcccagccaagacgaaggccccagcgaggccttcgtaaactgtgacgagaacagccggcttgtctccctgaccctgaacccagtgaccagggcagacgagggctggtactggtgtggagtgaagcagggccacttctatggagagactgcagctgtctatgtggcagttgaagagaagaaggtagcagggtcccgctatgtcagcccagcgaaggcagacgctgctcctgatgagaaggtgctagactctggtgtccgggagattgagaacaaagccattcaggatcccaggctttttgcagaggaaaaggtcgtggcagatacgggagatcaagctggtgggagcagagcatctgtggattccagcagctctgaggaacaaggtgggagctccaaagcgctggtctccactctggtgcccctgggcctggtgctggcactgggagccgtggttgtgggggtggccagagcccggcacaggaagaacgtcgaccgagtttcaatcagaagctacaggacagacattagcatgtcagacttcgagaactccagggaatttggagccaatgacaacatgggagcctcttcgatcactcaggagacatccctcggaggaaaagatgagtttgttgccacccctgagagcaccacggagaccaaagagcccaagaaggcaaaaagatcg

SEQ ID NO: 2 - protein, 243 aa, Macaca fascicularis
EKYWCKWSNTGCQTLPSQDEGPSEAFVNCDENSRLVSLTLNPVTRADEGWYWCGVKQGHFYGETAAVYVAVEEKKVAGSRYVSPAKADAAPDEKVLDSGVREIENKAIQDPRLFAEEKVVADTGDQAGGSRASVDSSSSEEQGGSSKALVSTLVPLGLVLALGAVVVGVARARHRKNVDRVSIRSYRTDISMSDFENSREFGANDNMGASSITQETSLGGKDEFVATPESTTETKEPKKAKRS

SEQ ID NO: 3 - protein, 663 aa, Streptococcus pneumoniae
ENEGSTQAATSSNMAKTEHRKAAKQVVDEYIEKMLREIQLDRRKHTQNVALNIKLSAIKTKYLRELNVLEEKSKDELPSEIKAKLDAAFEKFKKDTLKPGEKVAEAKKKVEEAKKKAEDQKEEDRRNYPTNTYKTLELEIAEFDVKVKEAELELVKEEAKESRNEGTIKQAKEKVESKKAEATRLENIKTDRKKAEEEAKRKADAKLKEANVATSDQGKPKGRAKRGVPGELATPDKKENDAKSSDSSVGEETLPSSSLKSGKKVAEAEKKVEEAEKKAKDQKEEDRRNYPTNTYKTLDLEIAESDVKVKEAELELVKEEAKEPRDEEKIKQAKAKVESKKAEATRLENIKTDRKKAEEEAKRKAAEEDKVKEKPAEQPQPAPATQPEKPAPKPEKPAEQPKAEKTDDQQAEEDYARRSEEEYNRLTQQQPPKTEKPAQPSTPKTGWKQENGMWYFYNTDGSMATGWLQNNGSWYYLNANGAMATGWLQNNGSWYYLNANGSMATGWLQNNGSWYYLNANGAMATGWLQYNGSWYYLNSNGAMATGWLQYNGSWYYLNANGDMATGWLQNNGSWYYLNANGDMATGWLQYNGSWYYLNANGDMATGWVKDGDTWYYLEASGAMKASQWFKVSDKWYYVNGSGALAVNTTVDGYGVNANGEWVN

SEQ ID NO: 4 - protein, 5 aa, Artificial Sequence
  Description of Artificial Sequence Illustrative conserved peptide among pIgR homologs
LRKED

SEQ ID NO: 5 - protein, 7 aa, Artificial Sequence
  Description of Artificial Sequence Illustrative conserved peptide among pIgR homologs
QLFVNEE

SEQ ID NO: 6 - protein, 5 aa, Artificial Sequence
  Description of Artificial Sequence Illustrative conserved peptide among pIgR homologs
LNQLT

SEQ ID NO: 7 - protein, 5 aa, Artificial Sequence
  Description of Artificial Sequence Illustrative conserved peptide among pIgR homologs
YWCKW

SEQ ID NO: 8 - protein, 5 aa, Artificial Sequence
  Description of Artificial Sequence Illustrative conserved peptide among pIgR homologs
GWYWC

SEQ ID NO: 9 - protein, 6 aa, Artificial Sequence
  Description of Artificial Sequence Illustrative conserved peptide among pIgR homologs
STLVPL

SEQ ID NO: 10 - protein, 5 aa, Artificial Sequence
  Description of Artificial Sequence Illustrative conserved peptide among pIgR homologs
SYRTD

SEQ ID NO: 11 - protein, 5 aa, Artificial Sequence
  Description of Artificial Sequence Illustrative conserved peptide among pIgR homologs
KRSSK

SEQ ID NO: 12 - DNA, 37 bp, Artificial Sequence
  Description of Artificial Sequence Primer
cgggaagatctggagtgaagcagggccacttctatgg

SEQ ID NO: 13 - DNA, 58 bp, Artificial Sequence
  Description of Artificial Sequence Primer
cggaattcctagtgatggtgatggtgatgtttggagctcccaccttgttcctcagagc

SEQ ID NO: 14 - protein, 22 aa, Artificial Sequence
  Description of Artificial Sequence PelB leader sequence
MKYLLPTAAAGLLLLAAQPAMA

SEQ ID NO: 15 - protein, 23 aa, Artificial Sequence
  Description of Artificial Sequence Synthetic carboxy terminal peptide
AAAEQKLISEEDLNGAAHHHHHH

SEQ ID NO: 16 - protein, 12 aa, Artificial Sequence
  Description of Artificial Sequence Linker peptide
GGGSGGGSGGGS

SEQ ID NO: 17 - protein, 6 aa, Artificial Sequence
  Description of Artificial Sequence 6-His tag
HHHHHH

SEQ ID NO: 18 - protein, 7 aa, Artificial Sequence
  Description of Artificial Sequence Illustrative pIgR peptide fragment
EKYWCKW

SEQ ID NO: 19 - protein, 8 aa, Artificial Sequence
  Description of Artificial Sequence Illustrative pIgR peptide fragment
DEGWYWCG

SEQ ID NO: 20 - protein, 20 aa, Artificial Sequence
  Description of Artificial Sequence Linker peptide
GGGGSGGGGSGGGGSGGGGS

SEQ ID NO: 21 - protein, 8 aa, Macaca fascicularis
EPKKAKRS

SEQ ID NO: 22 - protein, 6 aa, Macaca fascicularis
EPKKAK

SEQ ID NO: 23 - DNA, 28 bp, Artificial Sequence
  Description of Artificial Sequence Primer
gtatcgatctttttgccttcttgggytc

SEQ ID NO: 24 - DNA, 28 bp, Artificial Sequence
  Description of Artificial Sequence Primer
ggaattcgaraartaytggtgyaartgg

SEQ ID NO: 25 - DNA, 28 bp, Artificial Sequence
  Description of Artificial Sequence Primer
gtatcgatcnrttngcrttrttnggrtc

SEQ ID NO: 26 - protein, 6 aa, Artificial Sequence
  Description of Artificial Sequence Illustrative known epitope
QDPRLF

SEQ ID NO: 27 - protein, 54 aa, Artificial Sequence
  Description of Artificial Sequence Synthetic formula peptide
XXXXXXXXXXCXXXXXCXXXXXXXXXXXXXXXXXXXXCXXXXXCXXXXXXXXXX

SEQ ID NO: 28 - protein, 764 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 29 - protein, 757 aa, Bos taurus
MSRLFLACLLAIFPVVSMKSPIFGPEEVTSVEGRSVSIKCYYPPTSVNRHTRKYWCRQGAQGRCTTLISSEGYVSDDYVGRANLTNFPESGTFVVDISHLTHKDSGRYKCGLGISSRGLNFDVSLEVSQDPAQASHAHVYTVDLGRTVTINCPFTRANSEKRKSLCKKTIQDCFQVVDSTGYVSNSYKDRAHISILGTNTLVFSVVINRVKLSDAGMYVCQAGDDAKADKINIDLQVLEPEPELVYGDLRSSVTFDCSLGPEVANVPKFLCQKKNGGACNVVINTLGKKAQDFQGRIVSVPKDNGVFSVHITSLRKEDAGRYVCGAQPEGEPQDGWPVQAWQLFVNEETAIPASPSVVKGVRGGSVTVSCPYNPKDANSAKYWCHWEEAQNGRCPRLVESRGLIKEQYEGRLALLTEPGNGTYTVILNQLTDQDTGFYWCVTDGDTRWISTVELKVVQGEPSLKVPKNVTAWLGEPLKLSCHFPCKFYSFEKYWCKWSNRGCSALPTQNDGPSQAFVSCDQNSQVVSLNLDTVTKEDEGWYWCGVKEGPRYGETAAVYVAVESRVKGSQGAKQVKAAPAGAAIQSRAGEIQNKALLDPSFFAKESVKDAAGGPGAPADPGRPTGYSGSSKALVSTLVPLALVLVAGVVAIGVVRARHRKNVDRISIRSYRTDISMSDFENSRDFEGRDNMGASPEAQETSLGGKDEFATTTEDTVESKEPKKAKRSSKEEADEAFTTFLLQAKNLASAATQNGPTEA

SEQ ID NO: 30 - protein, 769 aa, Rattus norvegicus
MRLSLFALLVTVFSGVSTQSPIFGPQDVSSIEGNSVSITCYYPDTSVNRHTRKYWCRQGANGYCATLISSNGYLSKEYSGRASLINFPENSTFVINIAHLTQEDTGSYKCGLGTTNRGLFFDVSLEVSQVPEFPNDTHVYTKDIGRTVTIECRFKEGNAHSKKSLCKKRGEACEVVIDSTEYVDPSYKDRAILFMKGTSRDIFYVNISHLIPSDAGLYVCQAGEGPSADKNNADLQVLEPEPELLYKDLRSSVTFECDLGREVANDAKYLCRKNKETCDVIINTLGKRDPAFEGRILLTPRDDNGRFSVLITGLRKEDAGHYQCGAHSSGLPQEGRPVQAWQLFVNEESTIPNSRSVVKGVTGGSVAIVCPYNPKESSSLKYWCHWEADENGRCPVLVGTQALVQEGYEGRLALFEQPGSGAYTVILNQLTTQDSGFYWCLTDGDSRWRTTIELQVAEATKKPDLEVTPQNATAVIGETFTISCHYPCKFYSQEKYWCKWSNDGCHILPSHDEGARQSSVSCDQSSQIVSMTLNPVKKEDEGWYWCGVKEGQVYGETTAIYVAVEERTRGSPHINPTDANARAKDAPEEEAMESSVREDENKANLDPRLFADEREIQNAGDQAQENRASGNAGSAGGQSGSSKVLFSTLVPLGLVLAVGAVAVWVARVRHRKNVDRMSISSYRTDISMGDFRNSRDLGGNDNMGATPDTQETVLEGKDEIETTTECTTEPEESKKAKRSSKEEADMAYSAFLWQSSTIAAQVHDGPQEA

SEQ ID NO: 31 - protein, 771 aa, Mus musculus
MRLYLFTLLVTVFSGVSTKSPIFGPQEVSSIEGDSVSITCYYPDTSVNRHTRKYWCRQGASGMCTTLISSNGYLSKEYSGRANLINFPENNTFVINIEQLTQDDTGSYKCGLGTSNRGLSFDVSLEVSQVPELPSDTHVYTKDIGRNVTIECPFKRENVPSKKSLCKKTNQSCELVIDSTEKVNPSYIGRAKLFMKGTDLTVFYVNISHLTHNDAGLYICQAGEGPSADKKNVDLQVLAPEPELLYKDLRSSVTFECDLGREVANEAKYLCRMNKETCDVIINTLGKRDPDFEGRILITPKDDNGRFSVLITGLRKEDAGHYQCGAHSSGLPQEGWPIQTWQLFVNEESTIPNRRSVVKGVTGGSVAIACPYNPKESSSLKYWCRWEGDGNGHCPALVGTQAQVQEEYEGRLALFDQPGNGTYTVILNQLTTEDAGFYWCLTNGDSRWRTTIELQVAEATREPNLEVTPQNATAVLGETFTVSCHYPCKFYSQEKYWCKWSNKGCHILPSHDEGARQSSVSCDQSSQLVSMTLNPVSKEDEGWYWCGVKQGQTYGETTAIYIAVEERTRGSSHVNPTDANARAKVALEEEVVDSSISEKENKAIPNPGPFANEREIQNVRDQAQENRASGDAGSADGQSRSSSSKVLFSTLVPLGLVLAVGAIAVWVARVRHRKNVDRMSISSYRTDISMADFKNSRDLGGNDNMGASPDTQQTVIEGKDEIVTTTECTAEPEESKKAKRSSKEEADMAYSAFLLQSSTIAAQVHDGPQEA

SEQ ID NO: 32 - protein, 774 aa, Oryctolagus cuniculus
MALFLLTCLLAVFSAATAQSSLLGPSSIFGPGEVNVLEGDSVSITCYYPTTSVTRHSRKFWCREEESGRCVTLASTGYTSQEYSGRGKLTDFPDKGEFVVTVDQLTQNDSGSYKCGVGVNGRGLDFGVNVLVSQKPEPDDVVYKQYESYTVTITCPFTYATRQLKKSFYKVEDGELVLIIDSSSKEAKDPRYKGRITLQIQSTTAKEFTVTIKHLQLNDAGQYVCQSGSDPTAEEQNVDLRLLTPGLLYGNLGGSVTFECALDSEDANAVASLRQVRGGNVVIDSQGTIDPAFEGRILFTKAENGHFSVVIAGLRKEDTGNYLCGVQSNGQSGDGPTQLRQLFVNEEIDVSRSPPVLKGFPGGSVTIRCPYNPKRSDSHLQLYLWEGSQTRHLLVDSGEGLVQKDYTGRLALFEEPGNGTFSVVLNQLTAEDEGFYWCVSDDDESLTTSVKLQIVDGEPSPTIDKFTAVQGEPVEITCHFPCKYFSSEKYWCKWNDHGCEDLPTKLSSSGDLVKCNNNLVLTLTLDSVSEDDEGWYWCGAKDGHEFEEVAAVRVELTEPAKVAVEPAKVPVDPAKAAPAPAEEKAKAAVPSAQEKAVVPIVKEAENKVVQKPRLLAEEVAVQSAEDPASGSRASVDASSASGQSGSAKVLISTLVPLGLVLAAGAMAVAIARARHRRNVDRVSIGSYRTDISMSDLENSREFGAIDNPSACPDARETALGGKDELATATESTVEIEEPKKAKRSSKEEADLAYSAFLLQSNTIAAEHQDGPKEA

SEQ ID NO: 33 - protein, 243 aa, Homo sapiens
EKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRS

SEQ ID NO: 34 - protein, 243 aa, Macaca fascicularis; identical to SEQ ID NO: 2

SEQ ID NO: 35 - protein, 257 aa, Oryctolagus cuniculus
EKYWCKWNDHGCEDLPTKLSSSGDLVKCNNNLVLTLTLDSVSEDDEGWYWCGAKDGHEFEEVAAVRVELTEPAKVAVEPAKVPVDPAKAAPAPAEEKAKAAVPSAQEKAVVPIVKEAENKVVQKPRLLAEEVAVQSAEDPASGSRASVDASSASGQSGSAKVLISTLVPLGLVLAAGAMAVAIARARHRRNVDRVSIGSYRTDISMSDLENSREFGAIDNPSACPDARETALGGKDELATATESTVEIEEPKKAKRS

SEQ ID NO: 36 - protein, 94 aa, Homo sapiens
GVKQGHFYGETAAVYVAVEERKAAGSRDVSLAKADAAPDEKVLDSGFREIENKAIQDPRLFAEEKAVADTRDQADGSRASVDSGSSEEQGGSSR

SEQ ID NO: 37 - protein, 94 aa, Macaca fascicularis
GVKQGHFYGETAAVYVAVEEKKVAGSRYVSPAKADAAPDEKVLDSGVQEIENKAIQDPRLFAEEKVVADTGDQAGGSRASVDSSSSEEQGGSSK

SEQ ID NO: 38 - protein, 110 aa, Oryctolagus cuniculus
GAKDGHEFEEVAAVRVELTEPAKVAVEPAKVPVDPAKAAPAPAEEKAKAAVPSAQEKAVVPIVKEAENKVVQKPRLLAEEVAVQSAEDPASGSRASVDASSASGQSGSAK

SEQ ID NO: 39 - protein, 243 aa, Homo sapiens; identical to SEQ ID NO: 33

SEQ ID NO: 40 - protein, 243 aa, Macaca fascicularis; identical to SEQ ID NO: 2

SEQ ID NO: 41 - protein, 243 aa, Macaca fascicularis
EKYWCKWSNTGCQTLPSQDEGPSEAFVNCDEDSRLVSLTLNPVTRADEGWYWCGVKQGHFYGETAAVYVAVEEKKVAGSRYVSPAKADAAPDEKVLDSGVREIENKAIQDPRLFAEEKVVADTGDQAGGSRASVDSSSSEEQGGSSKALVSTLVPLGLVLALGAVVVGVARARHRKNVDRVSIRSYRTDISMSDFENSREFGANDNMGASSITQETSLGGKDEFVATPESTTETKEPKKAKRS

SEQ ID NO: 42 - protein, 290 aa, Artificial Sequence
  Description of Artificial Sequence Synthetic Pelb/5AF/myc/6His construct
MKYLLPTAAAGLLLLAAQPAMADYKAKQVQLVQSGGGVVQPGRSLRLSCAASGFTFSSYAMHWVRQAPGKGLEWVSAISGSGGSTYYADSVKGRFTISRDNAKNSLYLQMNSLRAEDTAVYYCARDTRGYFDLWGRGTLVTVSSGGGGSGGGGSGGGGSSELTQDPAMSVALGQTVRITCQGDSLRKYHASWYQQRPRQAPRLVVYGKNERPSGIPERFSGSTSGDTASLTISGLQAEDEADYYCHSRDSNADLVVFGGGTKVTVLGAAAEQKLISEEDLNGAAHHHHHH

SEQ ID NO: 43 - protein, 243 aa, Artificial Sequence
  Description of Artificial Sequence pIgR consensus sequence
EKYWCKWNNTGCQXLPSQDEGPSXAFVNCDENSRLVSLTLNXVTRADEGWYWCGVKQGHFYGETAAVYVAVEEXKVAGSRXVSPAKADAAPDEKVLDSGVREIENKAIQDPRLFAEEKAVADTXDQAXGSRASVDSSSSEEQGGSSKALVSTLVPLGLVLAXGAVAVGVARARHRKNVDRVSIRSYRTDISMSDFENSREFGANDNMGASSITQETSLGGKDEFVATTESTTETKEPKKAKRS

SEQ ID NO: 44 - protein, 94 aa, Artificial Sequence
  Description of Artificial Sequence pIgR stalk consensus sequence
GVKQGHFYGETAAVYVAVEEXKVAGSRXVSPAKADAAPDEKVLDSGVXEIENKAIQDPRLFAEEKAVADTXDQAXGSRASVDSSSSEEQGGSSK

SEQ ID NO: 45 - protein, 243 aa, Artificial Sequence; identical to SEQ ID NO: 2
  Description of Artificial Sequence pIgR consensus sequence

SEQ ID NO: 46 - protein, 250 aa, Artificial Sequence
  Description of Artificial Sequence Consensus sequence
EXXXXXXXXXXXXXXXXXXXXXXXXILVQEHGLPGPAQPRRRPQQGLRELRGQPACPDPEPGDQGRRGLVLVWSEAGPLLWRDCSLCGSREEGSGVPLCQPSEGRRCSEGALWFSGDEQSHSGSQAFCRGKGGGRYGRSRWEQSICGLQQLGTRWELQSAGLHSGAPPGAGSGSRGCGGGQSPAQEERRPSFNQXLDGHHVRLRELQGIWSQQHGSLFDHSGRPSEEKXEFXXPPXESTTXDQRTQXRQK

SEQ ID NO: 47 - protein, 248 aa, Homo sapiens
  MOD_RES (2)..(25): Variable amino acid
EXXXXXXXXXXXXXXXXXXXXXXXXILVQEHGLPGPAQPRRRPQQGLRELREQPACPDPEPGDQGGLVLVWSEAGPLLWRDCSLCGSREEGSGVPRCQPSEGRRCSEGALWFSGDEQSHSGSQAFCRGKGGGRYKRSRWEQSICGFRQLGTRWKLQSAGLHPGAPPGAGSGSRGCGGGQSPAQEERRPSFNQKLDRHHVRLRELQGIWSQQHGSLFDHSGRPSEEKXEFXXPPXESTTXDQRTQXRQK

SEQ ID NO: 48 - protein, 247 aa, Macaca fascicularis
  MOD_RES (192)..(192): Variable amino acid
EGGLELLERPPVWISAEFALGIRKILVQEHRLPDPAQPRRRPQRGLRKLRGQPACPDPEPSDQGRRGLVLVWSEAGPLLWRDCSLCGSREEGSRVPLCQPSEGRRCSEGALWCPGDEQSHSGSQAFCRGKGRGRYGRSWWEQSICGLQQLGTRWELQSAGLHSGAPPGAGTGSRGCGGGQSPEERRPSFNQXLDGHHVRLRELQGIWSQQHGSLFDHSGRPSEEKDEFCWPPPESTTGDQRTQVRQK

(19) United States
(12) Patent Application Publication
(10) Pub. No.: US 2004/0219542 A1
(43) Pub. Date: Nov. 4, 2004

(54) Compositions and methods for identifying, characterizing, optimizing and using ligands to transcytotic molecules

(76) Inventors: L.L. Houston, Bainbridge, WA (US); Philip L. Sheridan, San Diego, CA (US)

Correspondence Address: FOLEY & LARDNER, P.O. BOX 80278, SAN DIEGO, CA, 92138-0278, US

(21) Appl. No.: 10/470,987

(22) PCT Filed: Feb. 1, 2002
(86) PCT No.: PCT/US02/03059

(30) Foreign Application Priority Data
Feb. 2, 2001 (US) 60266182

(51) Int. Cl. C12Q 1/68

(52) U.S. Cl. 435/6

(57) ABSTRACT

The invention provides compositions and methods, including screening assays, for obtaining ligands (targeting elements) directed to a target molecule that undergoes apical endocytosis, reverse transcytosis, and/or basolateral exocytosis. Further provided are methods of identifying molecules that specifically bind a transcytotic molecule. Further provided are methods of identifying phage displaying a polypeptide, and determining the sequence of the polypeptide, that gives phage the ability to penetrate a layer of epithelial cells. Such polypeptides may confer paracellular transporting properties and/or transcytotic properties.

### FIELD OF THE INVENTION

The invention is drawn to compositions and methods for identifying, characterizing, distinguishing, derivitizing, optimizing and using compounds that are or comprise a ligand that binds a polyimmunoglubulin receptor (pIgR) molecule, a secretory component (SC) molecule, or a pIgR stalk molecule.

### BACKGROUND OF THE INVENTION

The following description of the background of the invention is provided simply as an aid in understanding the invention and is not admitted to describe or constitute prior art to the invention.

Therapeutic drugs can be introduced into the body using a variety of formulations and by various of routes of administration. For many reasons, a preferred route of administration is one that is non-invasive, i.e, does not involve any physical damage to the body. Generally, physical damage of this type results from the use of a medical device, such as a needle, to penetrate or breach a dermal surface or other external surface of an animal. Invasive routes of administration include, for example, surgical implants and injections. Injections can be intravascular, intrathecal or subcutaneous, all of which have undesirable features. Non-invasive routes of administration include uptake from the gastrointestinal tract as well as non-invasive parenteral (i.e, other than gastrointestinal) routes such as, e.g., inhalation therapy.

Presently, there are few, if any, formulations for the administration of proteins, a relatively new type of therapeutic drug. This is especially true in the case of non-invasive routes of administration and formulations therefor. Despite the enormous potential of therapeutic proteins, the lack of compositions and methods for the non-invasive administration of proteins has, depending on the particular protein in question, limited or prevented the medical use thereof.

Compounds are trafficked into, out from and within a cell by various molecules. “Endocytosis” is a general term for the process of cellular internalization of molecules, i.e, processes in which cells takes in molecules from their environment, either passively or actively. “Exocytosis” is a general term for processes in which molecules are passively or actively moved from the interior of a cell into the medium surrounding the cell. “Transcytosis” is a general term for processes in which molecules are transported from one surface of a cell to another.

Active endocytosis, exocytosis and transcytosis typically involve or are mediated by receptors, molecules that are at least partially displayed on the surface of cells. Receptors have varying degrees of specificity; some are specific for a single molecule (e.g., a receptor specific for epidermal growth factor; or a receptor that specifically recognizes Ca⁺⁺); some are semi-specific (e.g., a receptor that mediates the cellular internalization of many members of a family of cellular growth factors, or a receptor that recognizes Ca⁺⁺, Mg⁺⁺ and Zn⁺⁺); or of limited specificity (e.g., a receptor that mediates the cellular internalization of any phosphorylated protein, or a receptor that recognizes any divalent cation). Other types of molecules that can cause or influence the entry of molecules into cells include, e.g., cellular pores, pumps, and coated pits. Pores such as gated channels and ionophores form a channel that extends through the cellular membrane and through which certain molecules can pass. Cellular pumps exchange one type of molecule within a cell for another type of molecule in the cell's environment. Coated pits are depressions in the cellular surface that are “coated” with bristlelike structures and which condense to surround external molecules; the condensed coated pits then “pinch off” to form membrane-bound, coated vesicles within the cell.

Molecules that cause, influence or undergo endocytosis, exocytosis and/or transcytosis can do so constitutively, i.e, at all times, or regulated, i.e, for example, only under certain conditions or at specific times. Some such molecules can only mediate and/or undergo endocytosis, whereas some mediate and/or undergo transcytosis as well as endocytosis. Moreover, some such molecules are present in all or most cells (i.e, are ubiquitous), or are present mostly or only in certain tissues (i.e, are tissue-specific) or particular cell types.

The lack of compositions and methods causing, enhancing, mediating or regulating the endocytosis of therapeutic, diagnostic or analytical compounds and compositions hinders or prevents various uses of such compounds. In particular, the full therapeutic potential of many compounds could be realized if they were taken up by cells lining the gastrointestinal tract, as one could then formulate pills or tablets for the administration of therapeutic agents to patients. Typically, pills and other formulations for the oral delivery, and suppositories for the rectal delivery, of therapeutic agents to the gastrointestinal tract result in better patient compliance, and less use of medical resources, as opposed to other delivery modalities such as, e.g., intravenous administration. Similarly, the therapeutic potential of many compounds could be realized if they were taken up by cells lining the respiratory tract, including the nasal cavity; cells lining the gastrointestinal tract; vaginal surfaces; on dermal surfaces; and ocular surfaces and buccal surfaces (see Sayani et al., Crit. Rev. Ther. Drug Carrier Systems 13:85-184, 1996). Attempts to develop oral delivery formulations for proteins are discussed by Wang (J. Drug Targeting 4:195-232, 1996), Sinko et al. (Pharm. Res. 16:527, 1999) and Stoll et al. (J. Controlled Release 64:217-228, 2000).

In addition to the need for compositions and methods for the entry of biologically active molecules into cells, there is a further need for compositions and methods for causing, enhancing, mediating or regulating, or that control the direction of, transcytosis. Transcytosis is the general term given for processes whereby molecules, including biologically active molecules, move from one side or surface of a cell to another.

Furthermore, degradation and inefficient absorption of compounds delivered by conventional means further reduces the efficacy of those compounds. The ability to utilize alternative delivery pathways, target particular cells and tissues for delivery, improve the retention and absorption of compounds to be delivered, and protect the effective compound during delivery would be of significant import to the pharmaceutical and biopharmaceutical industries.

The above limitations vis-à-vis cellular transport of molecules are present both in vitro (e.g., in cellular cultures) and in vivo (e.g., in animals). Such limitations prevent or limit the therapeutic, diagnostic and/or analytical uses as of various compounds and compositions in an animal, including a mammal which may be a human. Such uses are described herein.

One example of a molecule that undergoes or mediates endocytosis, exocytosis as well as forward and reverse transcytosis is the polymeric immunoglobulin receptor (pIgR). The following information regarding pIgR is provided to assist in understanding the background of the invention.

Typically, pIgR molecules are displayed on epithelial cells. Epithelial cells line the interior of organs that have enclosed, semi-enclosed or compartmentalized spaces. The interior (e.g., canals, ducts, cavities, etc.) of such organs is genericaly referred to as the lumen. The lumen of a particular organ may have a specific name, e.g., the gastrointestinal lumen, pulmonary lumen, nasal lumen, nasopharyngeal lumen, pharyngeal lumen, buccal (within the mouth) lumen, sublingual (under the tongue) lumen, vaginal lumen, urogenital lumen, ocular lumen, or tympanic lumen. See, for example, Fahey et al., Immunol. Invest. 27:167-180, 1998; Brandtzaeg, J. Reprod. Immunol. 36:23-50, 1997; Kaushic et al., Biol. Reprod. 57:958-966, 1997; Richardson et al., J. Reprod. Immunol. 33:95-112; Kaushic et al., Endocrinology 136:2836-2844, 1995. Some of these might also be characterized as surfaces, e.g., the ocular surface.

Adjacent epithelial cells are connected by tight junctions. Disruption of tight junctions allows agents within the lumen, which often has an opening to the external environment of an animal, to penetrate into the body. Although such agents might include therapeutic agents, entry into the body via a disrupted tight junction is not specific; undesirable agents (e.g., bacteria, viruses, toxins and the like) will also be taken into the body. Due to this lack of specificity, as well as other factors, disruption of tight junctions for drug delivery purposes is generally not feasible and would, in any event, have many potential undesirable side effects.

Epithelial cells have two distinct surfaces: the apical side, which faces the lumen and is exposed to the aqueous or gaseous medium present therein; and an opposing basolateral (a.k.a. basal lateral) side that rests upon and is supported by an underlying basement membrane. The tight junctions between adjacent epithelial cells separate the apical and basolateral sides of an individual epithelial cell.

Epithelial cells are said to have polarity, that is, they are capable of generating gradients between the compartments they separate (for reviews, see Knust, Curr. Op. Genet. Develop. 10:471-475, 2000; Matter, Curr. Op. Genet. Develop. 10:R39-R42, 2000; Yeaman et al., Physiol. Rev. 79:73-98, 1999). This polarity reflects that fact that the cell has distinct plasma membrane domains (apical and basolateral) having distinct transport and permeability characteristics. For example, the apical side often contains microvilli for the adsorption of substances from the lumen, and, in ciliated cells, cilia are found on the apical membrane. As another example, the Na⁺/K⁺-ATPase pump is characteristically found only on the basolateral membrane.

FIG. 1 shows the pathways of cellular transport involving the pIgR protein, which undergoes or mediates endocytosis, exocytosis as well as forward and reverse transcytosis, in epithelial cells. Molecules of pIgR are typically displayed on the surfaces of epithelial cells and direct the trafficking of immunoglobulin (IgA) molecules. Other classes and species of immunoglobulins may also be trafficked. The right side of FIG. 1 illustrates the “forward” (i.e, basolateral to apical) transcytosis of pIgR molecules, whereas “reverse” (apical to basolateral) transeytosis is shown on the left side of the figure.

Forward transcytosis is the best characterized biological function of pIgR, and serves to convey protective antibodies (IgA and IgM immunoglobulins) from the circulatory system to the lumen of an organ. In forward transcytosis, pIgR molecules displayed on the basolateral side of the cell bind IgA molecules in the bloodstream, and pIgR:IgA complexes are then endocytosed, i.e, taken up into the cell and into a vesicle. The pIgR:IgA complexes are transported to the apical side of the cell, where they are displayed on the cell surface. Delivery of IgA into the lumen occurs when the pIgR portion of a pIgR:IgA complex is cleaved, i.e, undergo proteolysis. This event separates the pIgR molecule into two components: the “secretory component” (SC), which is released into the lumen, and which remains bound to IgA in order to protect IgA from degradation, and the “stalk,” which remains displayed, at least temporarily, on the apical surface of the cell.

Surprisingly, ligands bound to stalks displayed on the apical side of a cell can undergo reverse transcytosis, i.e, transcytosis in the opposite direction of forward transcytosis, i.e, from the apical side of a cell to its basolateral side. In reverse transcytosis, pIgR molecules or portions thereof move from the apical surfaces of cells that line the lumen of an organ to the basolateral surfaces of these cells. In theory, pIgR-mediated reverse transcytosis could be used to deliver agents from a lumen (e.g., the interior of the gut or the airways of the lung) to the circulatory system or some other interior system, organ, tissue, portion or fluid of the body including by way of non-limiting example the lymphatic system, the vitreous humor, etc. For example, as is shown in FIG. 1, a compound having a element that binds to a portion of pIgR that undergoes reverse transcytosis could, due to its association with the pIgR stalk, be carried to the basolateral side of a cell, where it would be contacted with and/or released into the bloodstream.

Evidence has been presented that forward transcytosis is mediated by a vesicular process (Apodaca et al., J. Cell Biol. 125:67-86, 1994; Mostov, Annu. Rev. Immunol. 12:63-84, 1994). Although not wishing to be bound by any particular theory, FIG. 1 shows a similar vesicular mediated transport mechanism for reverse transcytosis. FIG. 1 is not intended to imply that such a mechanism actually exists because evidence to this fact is not available; the vesicular nature of reverse transcytosis is only a hypothesis based on what is known about forward transcytosis.

The polyimmunoglobulin receptor (pIgR) is reviewed by Mostov and Kaetzel, Chapter 12 in: Mucosal Immunology, Academic Press, 1999, pages 181-211 (1999).

U.S. Pat. No. 6,020,161 to Wu et al. is drawn to pIgR polypeptides and polynucleotides that encode pIgR polypeptides.

U.S. Pat. No. 5,484,707 to Goldblum et al. is drawn to methods for monitoring organ rejection in an animal based on the concentration of the free secretory component of (SC) pIgR.

Published PCT patent applications WO 98/30592 and WO 99/20310, both to Hein et al., and U.S. Pat. No. 6,045,774 to Hiatt et al., are drawn to synthetic proteins that mimic IgA molecules and are thus associated with the proteolytically generated secretory component (SC) of pIgR.

U.S. Pat. No. 6,072,041 to Davis et al. is drawn to fusion proteins that are directed to the secretory component of pIgR. The compositions of Davis et al. are stated to be transported specifically from the basolateral surface of epithelial cells to the apical surface.

Ferkol et al., Am. J.Respir. Crit. Care Med. 161:944-951, 2000, is stated to describe a fusion protein consisting of a sFv directed to the secretory component (SC) of human pIgR and an human alpha (1)—antitrypsin.

U.S. Pat. No. 6,042,833 to Mostov et al. is drawn to a method by which a ligand that binds to a portion of a pIgR molecule is thereby internalized into, or transported across, a cell expressing or displaying pIgR. Mostov et al. describes “genetic fusions” and “fusion proteins” that include ricin A, poly-(L)-Lys, or a phage surface protein.

U.S. patent application Ser. No. 60/199,423 (attorney docket no. 030854.0008 entitled “Compositions Comprising Carriers and Transportable Complexes” by Houston, L. L.), filed Apr. 23, 2000, describes various pharmaceutical compositions that may be applied to compositions and methods of the present invention.

U.S. patent application Ser. No. 09/818,247 (attorney docket no. 18062E-000900 entitled “Ligands Directed to the Non-Secretory Component, Non-Stalk Region of pIgR and Methods of Use Thereof” by Mostov, Keith E., and Chapin, Steven J.), filed Mar. 27, 2000, describes the B region of pIgR and ligands directed to the B region of pIgR.

U.S. patent application Ser. No. 60/192,198 (attorney docket no. 18062E-003000US entitled “Anti-pIgR Antibodies With Improved Transcytosis by Mostov, Keith E., Chapin, Steven J., and Richman-Eisenstat, Janice), filed Mar. 27, 2000, is drawn to single chain Fv antibody fragments (sFv or scFv) directed to a specific epitope in the B region of pIgR. The application describes two single chain antibodies directed to pIgR named “sFv-5A” and “sFv-5AF,” the latter of which is used in the studies described in the Examples.

Zhang et al. (Cell 102:827-837, 2000) states that pIgR translocates bacteria (specifically, Streptococcus pneumoniae) across nasopharyngeal epithelial cells. The bacterial translocation is reported to occur in the apical to basolateral (reverse) direction.

U.S. patent application Ser. No. 60/237,929 (attorney docket No. 030854.0009 entitled “Genetic Fusions of pIgR Ligands and Biologically Active Polypeptides for the Delivery of Therapeutic and Diagnostic Proteins” by Houston, L. L., Glynn, Jacqueline M., and Sheridan, Philip L.), filed Oct. 2, 2000, is drawn to fusion proteins comprising pIgR ligands and biologically active polypeptides.

U.S. patent application Ser. Nos. 60/248,478 and 60/248,819 (attorney docket Nos. 030854.0009.PRV2 and 030854.0009.PRV3 entitled “Protein Conjugates of pIgR Ligands for the Delivery of Therapeutic and Diagnostic Proteins” by Houston, L. L., and Hawley, Stephen), filed Nov. 13, 2000 and Nov. 14, 2000 respectively, are drawn to protein conjugates comprising pIgR ligands and biologically active polypeptides.

### SUMMARY OF THE INVENTION

The invention provides compositions and methods, including screening assays, for obtaining ligands (targeting elements) directed to a target molecule that undergoes apical endocytosis, reverse transcytosis, and/or basolateral exocytosis, preferably in an epithelial cell. The ligands of the invention may be any type of molecular species, including but not limited to small molecules, nucleic acids, polypeptides, and derivatives and conjugates thereof. By “directed to” it is meant that the ligand specifically binds the target molecule. As is understood in the art, a ligand molecule specifically binds its target molecule when in preferably associates with its target molecule to such an extent that its association with other molecules in a collection will occur to an insignificant degree (e.g., less than about 5%, preferably less 1%, of non-target molecules will be bound to the ligand.

A “screening assay” is a selective assay designed to identify, isolate, and/or determine the structure of, compounds within a collection that have a preselected, typically desirable attribute, which may be a biological activity. Such assays include automated, semi-automated assays and HTS (high throughput screening) assays. By “identifying” it is meant that a compound having a desirable attribute is isolated, its chemical structure is determined (including without limitation determining the nucleotide and amino acid sequences of nucelic acids and polypeptides, respectively) the structure of and, additionally or alternatively, purifying).

A “target molecule” is any molecule or moiety that is desired to obtain ligands directed thereto. Preferred target molecules of the invention are called “pIgR targets” and include the pIgR, fragments thereof, preferably the pIgR stalk molecule, as well as oligopeptides containing amino acid sequences from pIgR, and pIgR domains or regions that undergo apical endocytosis, reverse transcytosis, and/or basolateral exocytosis. The term “pIgR target” includes any pIgR isoform, pIgR homolog, pIgR-like protein, as well as fragments, derivatives or conjugates of any of the preceding molecules that undergo apical endocytosis, reverse transcytosis, and/or basolateral exocytosis. Non-limiting examples of pIgR targets include oligopeptides of from 4, 5 or 6 to about 50 amino acids in length.

A “ligand” or “targeting element” is any molecule, compound or moiety that is directed to (specifically binds) a molecule to which it is targeted. As used herein, the terms “ligand” and “targeting element” are synonymous. A ligand may be any type of molecule that is capable of binding to a preselected target molecule. By way of non-limiting example, a ligand may be a small molecule, nucleic acid, a polypeptide, and derivatives and conjugates thereof.

In one aspect, the invention provides a method of identifying molecules that specifically bind a transcytotic molecule, comprising contacting a diverse collection of molecules with at least one transcytotic molecule under conditions where complexes comprising a transcytotic molecule and a ligand thereof can form, and identifying the molecules present in the complexes. In a related aspect, the invention provides a method of identifying biologically active molecules that specifically bind a transcytotic molecule, comprising contacting a diverse collection of molecules with at least one transcytotic molecule under conditions where complexes comprising a transcytotic molecule and a ligand thereof can form, and identifying biologically active small molecules present in the complexes.

A “collection” may be a “mixture” (2 to 47 members), a “pool” (48 to 5,000 members) or a “library” (48 or more members) of compounds. By “diverse” it is meant that greater than 50% of the compounds in a collection have chemical structures that are not identical to any other member of the collection. Preferably, greater than 75% of the compounds in a collection have chemical structures that are not identical to any other member of the collection, more preferably greater than 90% and most preferably greater than about 99%. In this and other methods of the invention,the act of contacting a diverse collection of potential ligands with at least one transcytotic molecule under conditions where complexes comprising a transcytotic molecule and a ligand can form can be repeated at least once and as many as about 50, preferably less than 20 times. Similarly, in this and other methods disclosed herein, complexes can be separated from unbound members of a collection of compounds prior to being identified or characterized.

In one aspect, the invention provides a method of identifying molecules that specifically bind a pIgR target molecule, comprising contacting a diverse collection of molecules with at least one pIgR target molecule under conditions where complexes comprising said pIgR target molecule and a ligand thereof can form, and identifying molecules present in the complexes. In a related aspect, the invention provides a method of identifying biologically active molecules that specifically bind a pIgR target molecule, comprising contacting a diverse collection of molecules with a pIgR target molecule under conditions where complexes comprising said pIgR target molecule and a ligand thereof can form, and identifying biologically active molecules present in the complexes.

The term “biologically active” (synonymous with “bioactive”) as it is used herein indicates that a molecule or moiety itself has a biological effect, or that it modifies, causes, promotes, enhances, blocks, reduces, limits the production or activity of, or reacts with or binds to an endogenous molecule that has a biological effect.

A “biological effect” may be but is not limited to one that stimulates or causes an immunreactive response; one that impacts a biological process in an animal; one that impacts a biological process in a pathogen or parasite; one that generates or causes to be generated a detectable signal; and the like. Biologically active conjugates may be used in therapeutic, prophylactic and diagnostic methods and compositions. Biologically active compounds act to cause or stimulate a desired effect upon an animal. Non-limiting examples of desired effects include, for example, preventing, treating or curing a disease or condition in an animal suffering therefrom; limiting the growth of or killing a pathogen in an animal infected thereby; augmenting the phenotype or genotype of an animal; stimulating a prophylactic immunoreactive response in an animal; or diagnosing a disease or disorder in an animal.

In one aspect, the invention provides methods for identifying compounds capable of undergoing apical endocytosis, apical to basolateral transcytosis, basolateral exocytosis and, additionally or alternatively, apical to basolateral transcytosis. Such compounds may additionally be capable of being delivered to an intercellular location.

In one aspect, the invention provides methods of identifying phage displaying a polypeptide, and determining the sequence of the polypeptide, that gives phage the ability to penetrate a layer of epithelial cells from the apical side thereof, comprising contacting a diverse collection of phage to the apical side of an epithelial cell layer, and recovering phage on the basolateral side thereof.

In one aspect, the invention provides methods of screening for a phage displaying a polypeptide that gives phage paracellular transporting properties, comprising contacting a diverse collection of phage to the apical side of an epithelial cell layer, and recovering phage on the basolateral side of the layer. A “paracellular transporting property” is an attribute that causes, promotes paracellular transport including, by way of non-limiting example, transport through the tight gap junctions found in epithelial cell layers.

In one aspect, the invention provides methods of screening for a phage displaying a polypeptide that gives said phage transcytotic properties, comprising contacting a diverse collection of phage to the apical side of an epithelial cell layer, and recovering phage on the basolateral side of the layer. A “transcytotic property” is an attribute that causes, promotes or enhances transcytosis.

In one version of this aspect, the apical side is in contact with a first medium that has a different composition than that of a second medium in contact with the basolateral side thereof; for example, the second fluid is serum or blood. In a related aspect, the invention provides a method of generating a collection of phage that comprise a focused library of polypeptides that gives the phage the ability to penetrate a layer of epithelial cells from the apical side thereof, comprising mutagenizing one or more phage identified according to the method of this aspect of the invention.

In the context of therapeutic applications of the invention, the term “biologically active” indicates that a molecule has an activity that impacts an animal suffering from a disease or disorder in a positive sense and/or impacts a pathogen or parasite in a negative sense. Thus, a biologically active molecule may cause or promote a biological or biochemical activity within an animal that is detrimental to the growth and/or maintenance of a pathogen or parasite; or of cells, tissues or organs of an animal that have abnormal growth or biochemical characteristics, such as cancer cells.

In the context of diagnostic applications of the invention, the term “biologically active” indicates that a molecule can be used for in vivo or ex vivo diagnostic methods and in diagnostic compositons and kits. For diagnostic purposes, a preferred biologically active protein is one that can detected, typically (but not necessarily) by virtue of comprising a detectable polypeptide. Antibodies to a fusion protein may also be used for its detection. In the latter instance, a preferred antibody is one that recognizes an epitope not present in either of the parent polypeptides from which the fusion protein is derived, e.g., an epitope corresponding to amino acid sequences present at the junction of the parent polypeptides.

In the context of prophylactic applications of the invention, the term “biologically active” indicates that a molecule induces or stimluates an immunoreactive response. In some preferred embodiments, the immunoreactive response is designed to be prophylactic, i.e, prevents infection by a pathogen. In other preferred embodiments, the immunoreactive response is designed to cause the immune system of an animal to react to the detriment of cells of an animal, such as cancer cells, that have abnormal growth or biochemical characteristics. In this application of the invention, a compositon comprising a fusion protein will be generally be formulated as a vaccine.

It will be understood by those skilled in the art that a given molecule may be biologically active in therapeutic, diagnostic and prophylactic applications. An agent that is described as being “biologically active in a cell” is one that has biological activity in vitro (i.e, in a cell culture) or in vivo (i.e, in the cells of an animal). A “biologically active component” of a fusion protein is a portion of a fusion protein that is biologically active once it is liberated from a fusion protein it being understood, however, that such a component may also be biologically active in the context of the fusion protein.

Specific examples of members that are not biologically active include elements that have no effect on biological functions but which are incorporated for ease of manipulation of the conjugate or member thereof such as, e.g., poly-(L)-lysine for the in vitro chemical conjugation of the fusion protein to another molecule; a polypeptide derived from a phage surface protein intended for fusion proteins to be used in vitro in phage display libraries; or a protein that serves as a carrier for another protein such as, e.g., KLH (keyhole limpet hemocyanin), which serves as a carrier for immunogenic proteins.

The compounds identified by the methods of the invention may be chemically linked or otherwise associated with any other molecule for a variety of purposes. For example, a ligand have transcytotic or paracellular transporting properties may be linked to a biologically active molecule in order to create a therapeutic agent, or a candidate or lead compound for the development of diagnostic or therapeutic agents. Thus, compounds comprising the ligands of the invention are within the scope of the invention. Such compounds are formulated into pharmaceutical compositions that, when present in the lumen of an organ, are capable of being apically endocytosed, apically to basolaterally transcytosed, basolaterally exocytosed, or delivered to an intra cellular location, or which otherwise penetrate epithelial barriers lining the lumen. Preferred organs are the gastrointestinal tract and the lungs. Thus, the invention also encompasses pharmaceutical compositions and medical devices comprising the compounds of the invention. Such compounds, compositions and devices may be used in methods to delivery a variety of therapeutic agents in the course of treating a disease, and such methods are within the scope of the invention.

Biologically active polypeptides are one preferred class of biologically active molecules. Preferred polypytides for incorporation into a composition according to the invention include by way of non-limiting example hormones, cytokines, antibodies, antibody fragments, T-cell receptors, enzymes, complement components, blood coagulation proteins, soluble receptor fragments, growth factors or polypeptide epitopes that may be used in vaccines. Preferred peptide hormones include insulin, growth hormone, luteinizing hormone, any follicle stimulating hormone, calcitonins, parathyroid hormone, enzymes, enzyme inhibitors and the like, although it is understood that any peptide or biologically active peptide can be employed in practicing the invention. Cytokines are proteins involved in signaling between cells during an immune response or involved in an inflammatory response. Lymphokines are a class of cytokines produced by lymphocytes. Representative cytokines and growth factors include, for example, interferons (IFNs; e.g., IFNα, IFNβ, and IFNγ); interleukins (including IL-1 through IL-15); and colony stimulating factors (e.g., those involved in the division and differentiation of bone marrow stem cells and their progeny, for example, stem cell factor (SCF), granulocyte colony stimulating factor (G-CSF), erythropoietin (EPO), granulocyte macrophage colony stimulating factor (GM-SCF), fibroblast growth factors (e.g. FGF1 and FGF2), PDGF, EDGF, various species of VEGF, NT3, and NGF, BDNF; factor VIII, factor IX and insulin-like growth factor.

The invention provides compositions and methods for assaying and characterizing compounds that are directed to a pIgR target or a transcytotic target. By “derivitizating” it is meant to prepare one or more derivatives of a compound or set of compounds, such as a mixture or library. By “optimizing” it is meant to identify compounds and derivatives thereof that are enhanced (or diminished) with regard to one or more preselected attributes. An “attribute” is any desirable or undesirable characteristic, activity or property of a molecule that can be quantitatively or qualitatively measured or estimated. Non-limiting examples of molecular attributes include absolute or relative molecular weight, complete or partial sequences (amino acid or nucleotide) of polypeptides and nucleic acids, enzymatic activity, ligand binding activity, protease resistance, serum stability, pI, antigenicity, ability to be formulated into various pharmaceutical compositions, ease of production, associated side effects, specificity and the like. Molecular attributes of particular interest include but are nor limited to parameters associated with one or more biological activities, and those associated with ligand:pIgR binding; antigenicity; histocompatibility; protease resistance; stability in serum or other bodily fluids or portions ex vivo, in vivo or in pharmaceutical compositions; half-life in vitro (e.g., in a pharmaceutical composition), ex vivo or in vivo; and the like. Such assaying and characterization may, but need not, be carried out in the course of identifying, derivitizing or optimizing compounds that are or comprise pIgR ligands.

The invention provides compositions and methods for chemically modifying (e.g., derivitizing, conjugating and the like) and optimizing compounds that are or comprise pIgR ligands. By “derivitizating” it is meant to prepare one or more derivatives of a compound or set of compounds, such as a mixture or library. By “optimizing” it is meant to identify compounds and derivatives thereof that are enhanced (or diminished) with regard to one or more desirable (or undesirable) attributes. As is explained in more detail herein, a preferred type of optimization process is designed to optimize the biological activity of lead compounds and drugs.

The summary of the invention described above is not limiting and other features and advantages of the invention will be apparent from the following detailed description of the preferred embodiments, as well as from the claims.

### BRIEF DESCRIPTION OF THE DRAWINGS

FIG. 1 shows forward and reverse transcytotic pathways of the polyimmunoglobulin receptor (pIgR) in epithelial cells.

FIG. 2 shows alignments of the amino acid sequences of pIgR homologs. FIG. 2A, alignment of human, bovine, rat, mouse and rabbit pIgR molecules; FIG. 2B, alignment of human, rabbit and simian pIgR molecules.; FIG. 2C, alignment of amino acid sequences from the pIgR stalk regions of human, rabbit and simian pIgR moelcules; 2D, alignment of human pIgR amino acid sequences with 2 clones of a simian pIgR molecule.

FIG. 3 shows the amino acid sequence (SEQ ID NO:2) of the secreted form of the ScFv 5AF encoded by pSyn5AF. Symbols: Pelb leader, a leader sequence that directs secretion from E. coli; FLAG, FLAG epitope; linker, amino acid sequence (GGGS)₃; myc, c-myc epitope; 6 HIS, 6×His tag; CDR, complementarity-determining region; FR, framework element; and the heavy and light chains of the scFv are indicated. The sequence of ScFv 5A is identical to that of Sc 5A with the exception that the 5th residue in the ScFv sequence is glutamine (Q) in 5A and leucine (L) in 5AF. The amino-terminal Pelb leader sequence is MKYLLPTAAAGLLLLAAQPAMA, and the carboxy terminal sequence is AAAEQKLISEEDLNGAAHHHHHH.

FIG. 4 shows nucleotide (SEQ ID NO:1) and amino acid (SEQ ID NO:2) sequences of a simian pIgR.

FIG. 5 shows the partial amino acid sequence of a bacterial adhesion protein, CbpA (SEQ ID NO:__). Emboldened and underlined amino acid sequences indicate amino acid sequences that bind, or contain an element that binds, pIgR.

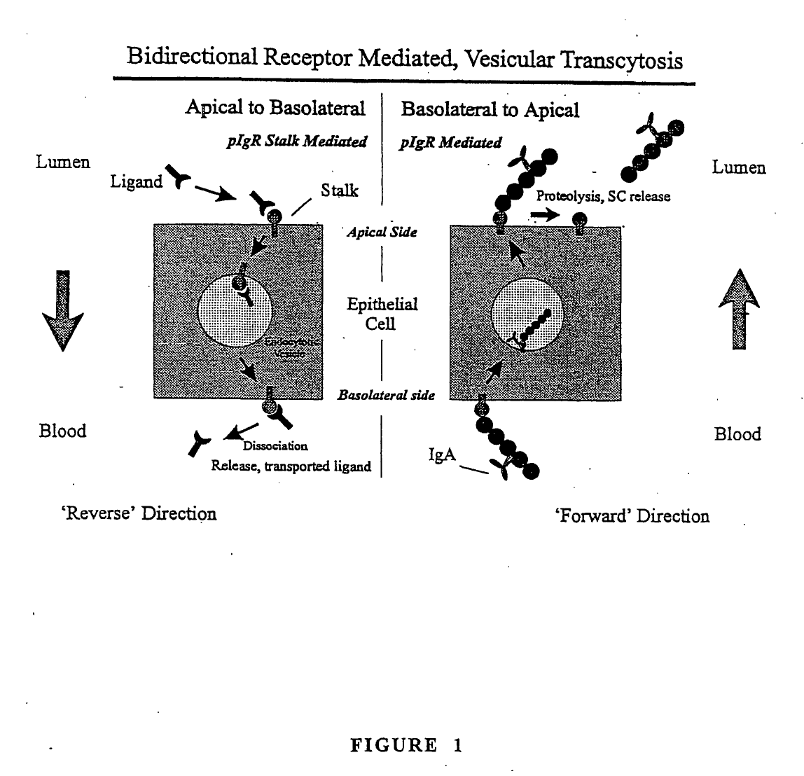

### DETAILED DESCRIPTION OF THE INVENTION

I. Introduction

The present invention provides compositions and methods for obtaining and characterizing compounds that are or comprise targeting elements (ligands) directed to target molecules such as pIgR and fragments thereof

Structure and Function of pIgR

A pIgR molecule has several structurally and functionally distinct regions that are defined as follows. It has been mentioned above that, in the art, a pIgR molecule is generally described as consisting of two different, loosely defined regions called the “stalk” and the “secretory component” (SC). A pIgR molecule binds polymeric immunoglobulins (IgA or IgM) on the basolateral side, and then transports the immunoglobulin to the apical side. Proteolyic cleavage of pIgR takes place on the apical side of an epithelial cell between the SC and the stalk, the former of which remains bound to and protects the immunoglobulins, and the latter of which remains bound to the apical membrane (see “Mucosal Immunoglobulins” by Mestecky et al. in: Mucosoal Immunology, edited by P. L. Ogra, M. E. Lamm, J. Bienenstock, and J. R. McGhee, Academic Press, 1999).

Particularly preferred pIgR molecule are those described in U.S. Pat. No. 6,042,833, the simian pIgR described herein, although it is understood that, in the context of this invention, pIgR also refers to any of that receptor's family or superfamily members, any homolog of those receptors identified in other organisms, any isoforms of these receptors, as well as any fragments, derivatives, mutations, or other modifications expressed on or by cells such as those located in the respiratory tract, the gastrointestinal tract, the urinary and reproductive tracts, the nasal cavity, buccal cavity, ocular surfaces, dermal surfaces and any other mucosal epithelial cells. Preferred pIgR and pIgR-like proteins are those that direct the endocytosis or transcytosis of proteins into or across epithelial cells.

As used herein, the terms “secretory component” and “SC” refers to the smallest (shortest amino acid sequence) portion of an apical proteolyzed pIgR molecule that retains the ability to bind immunoglobulins (IgA and IgM). After proteolytic cleavage of pIgR, some amino acid residues remain associated with SC:immunoglobulin complexes but are eventually degaraded and/or removed from such complexes (Ahnen et al., J. Clin. Invest. 77:1841-1848, 1986). According to the definiton of the secretory component used herein, such amino acids are not part of the SC. In certain embodiments of the invention, pIgR-targeting elements that do not recognize or bind to the SC are preferred.

Another way in which different portions of a pIgR molecule can be delineated is by reference to the domains thereof. A protein “domain” is a relatively small (i.e., <about 150 amino acids) globular unit that is part of a protein. A protein may comprise two or more domains that are linked by relatively flexible stretches of amino acids. In addition to having a semi-independent structure, a given domain may be largely or wholly responsible for carrying out functions that are normally carried out by the intact protein. In addition to domains that have been determined by in vitro manipulations of protein molecules, it is understood in the art that a “domain” may also have been identified in silico, i.e, by software designed to analyze the amino acid sequences encoded by a nucleic acid in order to predict the limits of domains. The latter type of domain is more accurately called a “predicted” or “putative” domain but, in the present disclosure, the term domain encompasses both known and predicted domains unless stated otherwise.

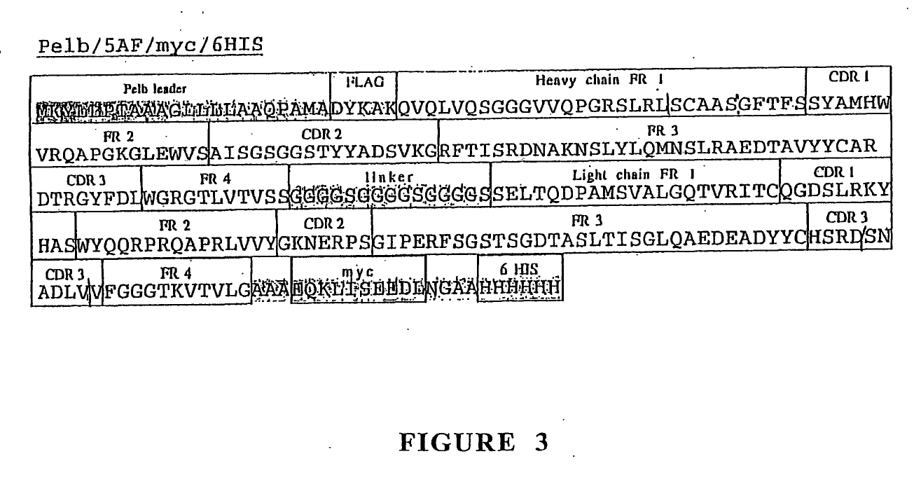

Extracellular domains 1 through 6 of pIgR molecules from several species are indicated in FIG. 3 of Piskurich et al. (J. Immunol. 154:1735-1747, 1995). In rabbit pIgR, domains 2 and 3 are encoded by a single exon that is sometimes deleted by alternative splicing. A transmembrane domain is also present in pIgR, as is an intracellular domain. The intracellular domain contains signals for transcytosis and endocytosis. Domains of a pIgR molecule that are of particular interest in the present disclosure include but are not limited to domain 5, domain 6, the transmembrane domain and the intracellular domain.

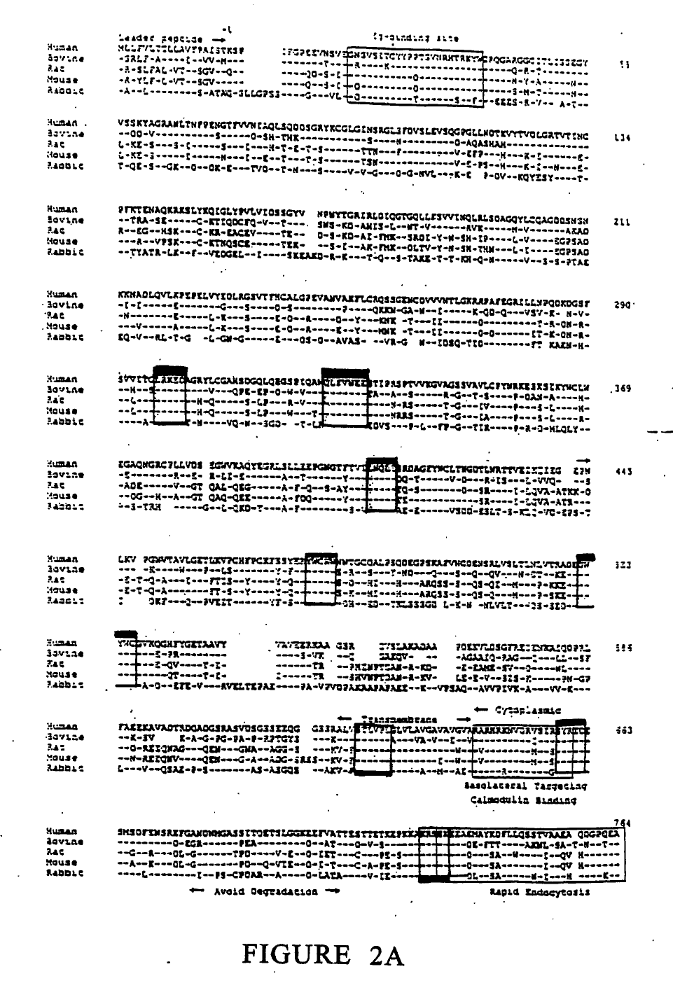

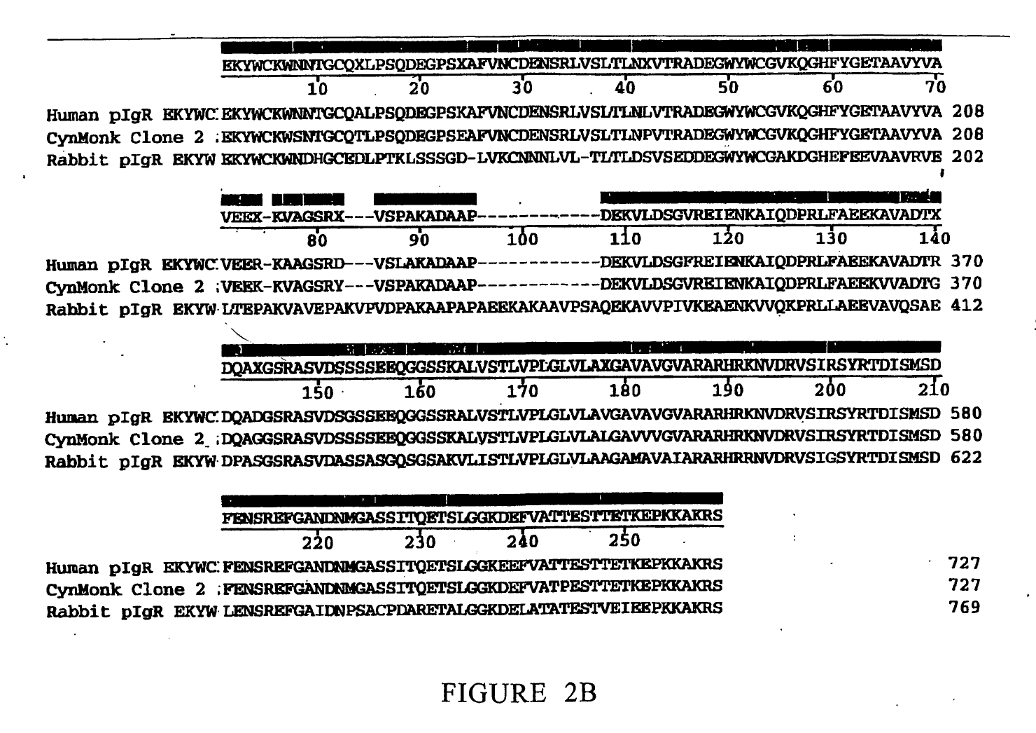

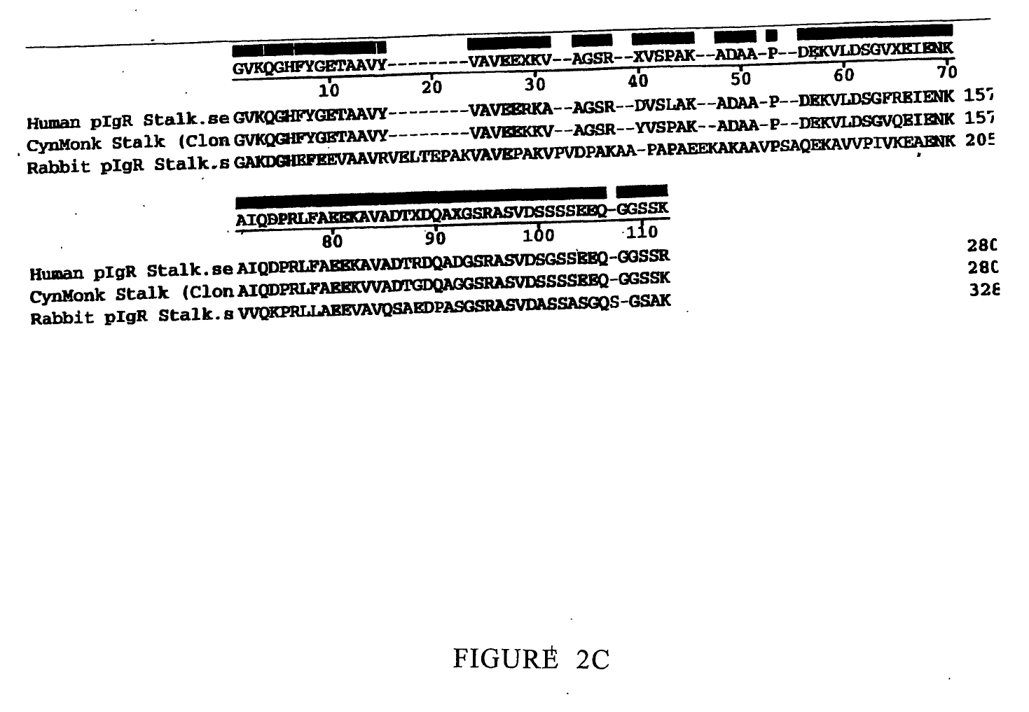

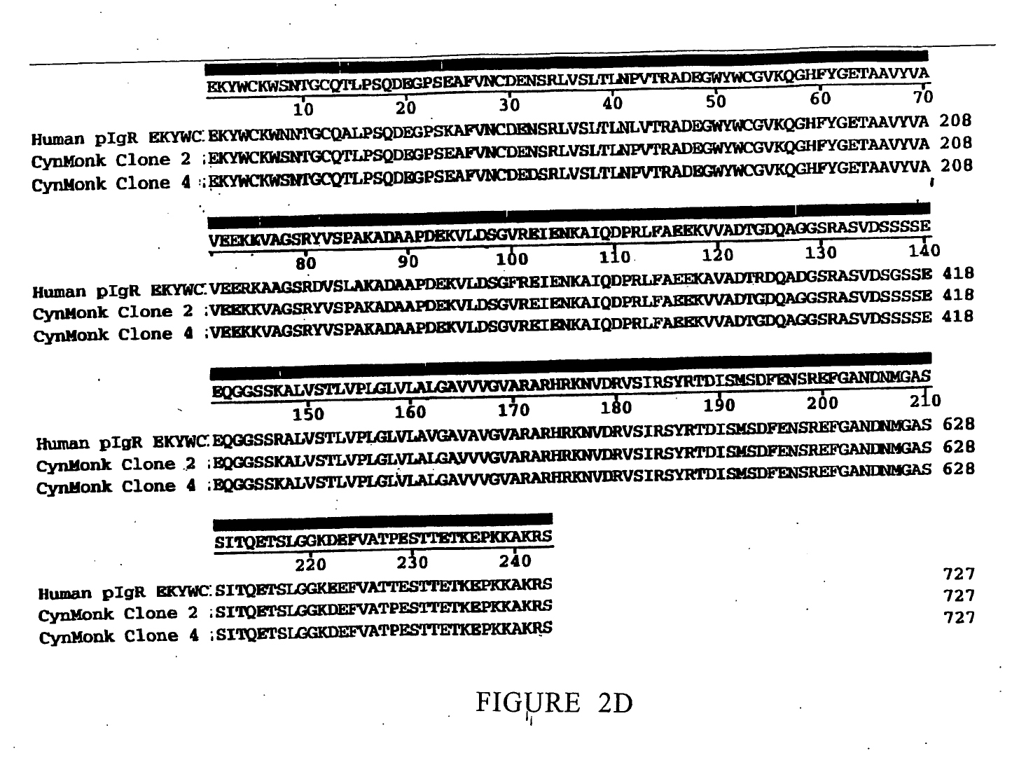

Another way in which different portions of a pIgR molecule can be defined is by reference to amino acid sequences that are conserved between pIgR homologs (i.e, pIgR molecules isolated from non-human species; see below). Non-limiting examples of conserved amino acid sequences include those found in Table 2; see also FIG. 2. (For brevity's sake, the one letter abbreviations for amino acids is used in Table 2, but a version of each sequence that employs the three letter amino acid designations may be found in the Sequence Listing; see also Table 1.)

TABLE 1
Abbreviations for Amino Acids

| | Three-letter | One-letter |
|---|---|---|
| Amino acid | Abbreviation | symbol |
| Alanine | Ala | A |
| Arginine | Arg | R |
| Asparagine | Asn | N |
| Aspartic Acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE 2
Amino Acid Sequences that are Conserved in pIgR Homologs

| | Position of Amino | |
|---|---|---|
| Amino Acid Sequence | Acid Residues in Human | |
| Conserved among pIgR | pIgR Relative to Amino | |
| Homologs | Terminal Methionine* | SEQ ID NO: |
| LRKED | 297-301, inclusive | |
| QLFVNEE | 325-331, inclusive | |
| LNQLT | 410-414, inclusive | |
| YWCKW | 476-480, inclusive | |
| GWYWC | 522-526, inclusive | |
| STLVPL | 624-629, inclusive | |
| SYRTD | 658-662, inclusive | |
| KRSSK | 732-737, inclusive | |

*As described in FIG. 3 of Mostov and Kaetzel, Chapter 12 in: Mucosal Immunology, Academic Press, 1999, pages 181-211.

Thus, for example, a specific internal portion of a given pIgR molecule might be defined as a region that has an amino-terminal border that has the amino acid sequence EKYWCKW and a carboxy-terminal border having the amino acid sequence side having the amino acid sequence DEGWYWCG. In human pIgR, the region so defined would be the amino acid sequence of residues 474 through 529. In the present invention, regions of any given pIgR molecule that are of particular interest include but are not limited to the following regions that are not conserved between pIgR homologs from different species:

R1 From KRSSK to the carboxy terminus,

R2a From SYRTD to the carboxy terminus,

R2b From SYRTD to KRSSK,

R3a From STLVPL to the carboxy terminus,

R3b From STLVPL to KRSSK,

R3c From STLVPL to SYRTD,

R4a From GWYWC to the carboxy terminus,

R4b From GWYWC to KRSSK,

R4c From GWYWC to SYRTD,

R4d From GWYWC to STLVPL,

R5a From YWCKW to the carboxy terminus,

R5b From YWCKW to KRSSK,

R5c From YWCKW to SYRTD,

R5d From YWCKW to STLVPL,

R5e From YWCKW to GWYWC,

R6a From LNQLT to the carboxy terminus,

R6b From LNQLT to KRSSK,

R6c From LNQLT to SYRTD,

R6d From LNQLT to STLVPL,

R6e From LNQLT to GWYWC,

R6f From LNQLT to YWCKW,

R7a From QLFVNEE to the carboxy terminus,

R7b From QLFVNEE to KRSSK,

R7c From QLFVNEE to SYRTD,

R7d From LNQLT to STLVPL,

R7e From QLFVNEE to GWYWC,

R7f From QLFVNEE to YWCKW,

R7g From QLFVNEE to LNQLT,

R8a From LRKED to the carboxy terminus,

R8b From LRKED to KRSSK,

R8c From LRKED to SYRTD,

R8d From LRKED to STLVPL,

R8e From LRKED to GWYWC,

R8f From LRKED to YWCKW,

R8g From LRKED to LNQLT, and

R8h From LRKED to QLFVNEE.

Homologs of pIgR are also within the scope of the invention. Homologs of pIgR are pIgR proteins from species other than Homo sapiens. By way of non-limiting example, pIgR proteins from various species include those from humans, the rat, mouse, rabbit, cow and possum (Table 3). See also FIG. 3 in Mostov and Kaetzel, Chapter 12, “Immunoglobulin Transport and the Polymeric Immunoglobulin Receptor” in Mucosal Immunity, Academic Press, 1999, pages 181-211; and Piskurich et al., J. Immunol. 154:1735-1747, 1995).

TABLE 3
pIgR and pIgR-like Proteins From Non-Human Species

| Organism | Accession Number(s) |
|---|---|
| Zebrafish (Brachydanio | 9863256, 8713834, 8282255, & 7282118 |
| rerio) | |
| Mouse (Mus musculus) | 8099664, 2804245, 6997240, 4585867, |
| | 4585866, 2688814, 2688813, 2688812, |
| | 2688811, 2688810, 2688809, 2688808, |
| | 2688807, 3097245, 3046754, 3046752, |
| | 3046751, 3046756, 3046755, 3046750, |
| | 3046748, 3046747 and 2247711 |
| Rat (Rattas norvegicus) | 2222806, 475572, 475571, 473408, 603168 |
| | and 603167 |
| Cow (Bos taurus) | 388279 |
| Possum (Trichosuras | 5305520, 5305518, 5305514 and 5305512 |
| vulpecula) | |

Also within the scope of the invention are pIgR-like proteins. A “pIgR-like protein” is a protein that has an amino acid sequence having homolgy to a known pIgR protein. In many instances, the amino acid sequences of such pIgR-like molecules have been generated by the in silico translation of a nucleic acid, wherein the nucleotide sequence of the nucleic acid has been determined but is not known to encode a protein. By way of non-limiting example, pIgR-like proteins include PIGRL1 (U.S. Pat. No. 6,114,515); a mouse gene having an exon similar to one of pIgR's (GenBank Accession No. 6826652); and human proteins translated in silico that have homology to pIgR proteins (GenBank Accession Nos. 1062747 and 1062741).

Substantially Identical and Homologous pIgR Molecules

As used herein, a “homolog” of a pIgR protein or a pIgR-like protein is a protein is an isoform or mutant of human pIgR, or a protein in a non-human species that either (i) is “identical” with or is “substantially identical” (determined as described below) to an amino acid sequence in human pIgR, or (ii) is encoded by a gene that is identical or substantially identical to the gene encoding human pIgR. Non-limiting examples of types of pIgR isoforms include isoforms of differing molecular weight that result from, e.g., alternate RNA splicing or proteolytic cleavage; and isoforms having different post-translational modifications, such as glycosylation; and the like.

Two amino acid sequences are said to be “identical” if the two sequences, when aligned with each other, are exactly the same with no gaps, substitutions, insertions or deletions. Two amino acid sequences are defined as being “substantially identical” if, when aligned with each other, (i) no more than 30%, preferably 20%, most preferably 15% or 10%, of the identities of the amino acid residues vary between the two sequences; or (ii) the number of gaps between or insertions in, deletions of and subsitutions of, is no more than 10%, preferably 5%, of the number of amino acid residues that occur over the length of the shortest of two aligned sequences. The entire amino acid sequence of two proteins may be substantially identical to one another, or sequences within proteins may demonstrate identity or substantial identity with sequences of similar length in other proteins. In either case, such proteins are substantially identical to each other. Typically, stretches of identical or substantially identical sequences occur over 5 to 25, preferably 6 to 15, and most preferably 7 to 10, nucleotides or amino acids.

One indication that nucleotide sequences encoding pIgR proteins are substantially identical is if two nucleic acid molecules hybridize to each other under stringent conditions. Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is about 0.02 M at pH 7 and the temperature is at least about 60° C.

Another way by which it can be determined if two sequences are substantially identical is by using an appropriate algorithm to determine if the above-described critera for substantially identical sequences are met. Sequence comparisons between two (or more) polynucleotides or polypeptides are typically performed by algorithms such as, for example, the local homology algorithm of Smith and Waterman (Adv. Appl. Math. 2:482, 1981), by the homology aligument algorithm of Needleman and Wunsch (J. Mol. Biol. 48:443, 1970), by the search for similarity method of Pearson and Lipman (Proc. Natl. Acad. Sci. U.S.A. 85:2444, 1988), by computerized implementations of these algorithms (GAP, BESTFIT, FASTA, and TFASTA in the Wisconsin Genetics Software Package, Genetics Computer Group (GCG), 575 Science Dr., Madison, Wis.), or by visual inspection.

pIgR Ligands

As used herein, the terms “ligand” and “targeting element” are synonymous and encompasses any type of molecule that is capable of binding to a preselected target molecule. A ligand may, by way of non-limiting example, be a small molecule, a nucleic acid, a polypeptide, and derivatives and conjugates thereof. The terms “ligand” and “targeting element” encompass any type of molecule or moiety that is capable of binding to its target moelcule, and a “pIgR ligand” or “pIgR targeting element” encompasses molecules and moieties that can bind pIgR or a fragment thereof. Preferred fragments are the pIgR stalk molecule and oligopeptides containing amino acid sequences from pIgR

Any ligand that binds pIgR to an effective degree is identifiable using the compositions and methods of the invention. By way of non-limiting example, a pIgR ligand may be an antibody; a bacterial protein that binds pIgR; a polypeptide identified by various methods described herein; a nucleic acid such as an aptamer; a small molecule identified by various methods described herein; a derivative of any of the preceding ligands; or a conjugate or mixture of two or more of the preceding ligands or derivatives thereof.

The binding of a ligand is target-specific in the sense that, although other molecules may be present in a mixture in which ligands and target molecules are contacted with each other, the ligand does not appreciably bind to other (non-target) molecules.

It is recognized that the strength of binding between pIgR and a pIgR ligand, i.e., the affinity of a pIgR ligand for pIgR, is a matter of degree. As used herein, “target-specific” means that the pIgR ligand has a stronger affinity for its target molecule (pIgR) than for contaminating molecules, and this difference in affinity is sufficient for a given aspect of the invention. In general, the target specificity of a pIgR ligand for pIgR is comparable to the specificity of antibodies for their antigens. Thus, by way of non-limiting example, the specificity for a ligand for pIgR should be at least approximately that of a single chain antibody (sFv) for pIgR. Examples of sFv's that can be used to evaluate the target specificity of a pIgR ligand include but are not limited to sFv-5A and derivatives thereof, such as sFv-5AF, which bind to the stalk of pIgR and are described herein; and sFv's that bind to the secretory component (SC) such as, e.g., those described in U.S. Pat. No. 6,072,041.

The specificity of the binding is defined in terms of the values of absolute and relative binding parameters, such as the comparative dissociation constants (Kd) of a ligand for its target molecule as compared to the dissociation constant with respect to the ligand and unrelated molecules and compositions. Typically, the Kd of a ligand with respect to its target molecule will be 2-fold, preferably 5-fold, more preferably 10-fold less, than the Kd of the ligand for unrelated molecules and compositions. Even more preferably the Kd will be 50-fold less, more preferably 100-fold less, and more preferably 200-fold less.

The binding affinity of the ligands with respect to target molecules is defined in terms of the dissociation constant (Kd). The value of Kd can be determined directly by well-known methods, and can be computed even for complex mixtures by methods such as those, for example, set forth in Caceci, M., et al., Byte (1984) 9:340-362. In some situations, direct determination of Kd is problematic and can lead to misleadingly results. Under such circumstances, a competitive binding assay can be conducted to compare the affinity of a ligand for its target molecule with the affinity of molecules known to bind the target molecule. The value of the concentration at which 50% inhibition occurs (Ki) is, under ideal conditions, roughly equivalent to Kd. Moreover, Ki cannot be less than Kd; determination of Ki sets a maximal value for the value of Kd. Under circumstances where technical difficulties preclude accurate measurement of Kd, measurement of Ki can conveniently be substituted to provide, at the very least, an upper limit for Kd.

Kd may be measured in solution using techniques and compositions described in the following publications. Blake, D. A.; Blake, R. C.; Khosraviani, M.; Pavlov, A. R. “Immunoassays for Metal Ions.” Analytica Chimica Acta 1998, 376, 13-19. Blake, D. A.; Chakrabarti, P.; Khosraviani, M.; Hatcher, F. M.; Westhoff, C. M.; Goebel, P.; Wylie, D. E.; Blake, R. C. “Metal Binding Properties of a Monoclonal Antibody Directed toward Metal-Chelate Complexes.” Journal of Biological Chemistry 1996, 271(44), 27677-27685. Blake, D. A.; Khosraviani, M.; Pavlov, A. R.; Blake, R. C. “Characterization of a Metal-Specific Monoclonal Antibody.” Aga, D. S.; Thurman, E. M., Eds.; ACS Symposium Series 657; American Chemical Society: Washington, DC, 1997; pp 49-60.

Kd is measured using immobilized binding components on a chip, for example, on a BIAcore chip using surface plasmon resonance. Surface plasmon resonance is used to characterize the microscopic association and dissociation constants of reaction between sFv directed against pIgG associated molecules and pIgR and pIgR fragments. Such general methods are described in the following references and are incorporated herein by reference (Vely F. Trautmann A. Vivier E., BIAcore analysis to test phosphopeptide-SH2 domain interactions, Methods in Molecular Biology. 121:313-21, 2000; Liparoto S F. Ciardelli T L., Biosensor analysis of the interleukin-2 receptor complex, Journal of Molecular Recognition. 12:316-21, 1999; Lipschultz C A. Li Y. Smith-Gill S., Experimental design for analysis of complex kinetics using surface plasmon resonance, Methods. 20):310-8, 2000; Malmqvist M., BIACORE: an affinity biosensor system for characterization of biomolecular interactions, Biochemical Society Transactions. 27:335-40, 1999; Alfthan K., Surface plasmon resonance biosensors as a tool in antibody engineering, Biosensors & Bioelectronics. 13:653-63, 1998; Fivash M. Towler E M. Fisher R J., BIAcore for macromolecular interaction, Current Opinion in Biotechnology. 9:97-101, 1998; Price M R. Rye P D. Petrakou E. Murray A. Brady K. Imai S. Haga S. Kiyozuka Y. Schol D. Meulenbroek M F. Snijdewint F G. Von Mensdorff-Pouinly S. Verstraeten R A. Kenemans P. Blockzjil A. Nilsson K. Nilsson O. Reddish M. Suresh M R. Koganty R R. Fortier S. Baronic L. Berg A. Longenecker M B. Hilgers J. et al.; Summary report on the ISOBM TD-4 Workshop: analysis of 56 monoclonal antibodies against the MUC1 mucin. San Diego, Calif., Nov. 17-23, 1996, Tumour Biology. 19 Suppl 1:1-20, 1998; Malmqvist M. Karlsson R, Biomolecular interaction analysis: affinity biosensor technologies for functional analysis of proteins, Current Opinion in Chemical Biology. 1:378-83, 1997; O'Shannessy D J. Winzor D J., Interpretation of deviations from pseudo-first-order kinetic behavior in the characterization of ligand binding by biosensor technology, Analytical Biochemistry. 236:275-83, 1996; Malmborg A C. Borrebaeck C A, BIAcore as a tool in antibody engineering, Journal of Immunological Methods. 183:7-13, 1995; Van Regenmortel M H., Use of biosensors to characterize recombinant proteins, Developments in Biological Standardization. 83:143-51, 1994; O'Shannessy D J., Determination of kinetic rate and equilibrium binding constants for macromolecular interactions: a critique of the surface plasmon resonance literature, Current Opinions in Biotechnology. 5:65-71, 1994). Additionally or alternatively, binding constants and kinetic constants are estimated using calorimetry, equilibrium dialysis, and stopped flow methods using absorbance, fluorsescence, light scattering, turbidity, fluorescence anisotropy, and the like.

pIgR Binding Assays

The ability of a pIgR ligand of the invention to bind different pIgR molecules, fragments and derivaties thereof, and to undergo endocytosis, transcytosis, and/or exocytosis is an important attribute of these proteins. The pIgR-binding capacity of fusion proteins are examined using the following techniques. Such assays include the following:

Ex Vivo Testing of Ligand Binding

The ex vivo pIgR binding capacity of a pIgR-targeted protein is assessed by measuring endocytosis or transcytosis of bound ligand in mammalian epithelial cells. Receptor-mediated endocytosis provides an efficient means of causing a cell to ingest material which binds to a cell surface receptor. (See Wu et al., J. Biol. Chem. 262:4429-4432, 1987; Wagner et al., Proc. Natl. Acad. Sci. USA 87:3410-3414, 1990, and published EPO patent application EP-A1 0388758). Any number of well known methods for assaying endocytosis may be used to assess binding. For example, binding, transcytosis, and internalization assays are described at length in Breiftfeld et al. (J. Cell Biol. 109:475-486, 1989).

Ligand-pIgR binding is measured by a variety of techniques known in the art, e.g., immunoasays and immunoprecipitation. By way of example, antibodies to the biologically active portion of a protein conjugate can be used to bind and precipitate detectably labeled pIgR molecules; the amount of labeled material that is precipitated corresponds to the degree of pIgR binding to a ligand such as, e.g., a protein conjugate having a pIgR-targeting element (see Tajima, J. Oral Sci. 42:27-31, 2000).

Apical Endocytosis is conveniently measured by binding a ligand such as a Fab fragment to the stalk at the apical surface of Madin-Darby canine kidney (MDCK) cells at 4° C., warming to 37° C. for brief periods (0-10 min), and cooling the cells back down to 4° C. Methods of pIgR expression in MDCK cells are known in the art (Breitfeld et al., Methods in Cell Biology 32:329-337, 01989). Fab remaining on the surface are removed by stripping at pH 2.3. Intracellular Fab molecules are those that remain cell-associated after the stripping, while surface-bound Fab are those removed by the acid wash. Controls for non-specific sticking include using pre-immune Fab and/or MDCK cells that are not transfected with pIgR.

Apical to Basolateral (“Reverse”) Transcytosis is assessed by allowing MDCK cells to bind the Fab at the apical surface at 4° C., warming up to 37° for 0 to 240 min, and then measuring the amount of Fab delivered into the basolateral medium. This basolaterally-delivered Fab is compared to the sum of Fab that remains associated with the cells (intracellular or acid-stripped) and the Fab released back into the apical medium. Alternatively, transcystosis is assessed by continuously exposing cells to the Fab in the apical medium and measuring accumulation of Fab in the basolateral medium. This method avoids cooling the cells, but does not provide the kinetics of transporting a single cohort of ligand. In both methods degradation of the Fab can be assessed by running aliquots of the transcytosed Fab on SDS-PAGE and probing a Western with antibodies.

Basolateral Endocytosis is assessed by methods such as those described by Tajima (J. Oral Sci. 42:27-31, 2000). Non-specific transport (e.g. due to fluid phase endocytosis and transcytosis, or paracellular leakage between cells) can be controlled for by using MDCK cells that are not transfected with the pIgR and/or pre-immune Fab.

Testing of Ligand Binding in vivo

In vivo Transcytosis is assessed using pathogen-free experimental animals such as Sprague-Dawley rats. Detectably labeled ligand (e.g., a radioiodinated antibody) is administered into, e.g., the nares (the pair of openings of the nose or nasal cavity of a vertebrate) or the intestine (see Example 8). As will be understood by those of skill in the art, a “detectable label” is a composition or moiety that is detectable by spectroscopic, photochemical, biochemical, immunochemical, electromagnetic, radiochemical, or chemical means such as fluorescence, chemifluoresence, or chemiluminescence, or any other appropriate means

In vivo Apical to Basolateral (“Reverse”) Transcytosis is assessed by measuring the delivery of a pIgR-targeting protein into the circulation as measured by the presence of a detectable label that has been incorporated into the protein that is being tested. The integrity of the ligand recovered from the circulation can be assessed by analyzing the ligand on SDS polyacrylamide gel electrophoresis.

II. Compound Libraries, Screening Assays and Compound Optimization

In brief, ligands are identified, isolated from, or have their structure determined from collections (libraries, pools, mixtures) of compounds, wherein some of the compounds in the collection are not ligands. These candidate ligands are derivitized, conjugated or other wise modified, or are used to provide structural information in order to generate lead compounds, pharmacophores and drugs. “Structural information” includes by way of non-limiting example, nucleotiode and amino acid sequences, chemical formulae, three-dimesional (3D) models of the atoms found at an active site and interactions between such atoms. By “active site” it is meant that portion of a ligand that binds a target.

Compound Libraries

A “compound library” or “library” is a collection of different compounds, i.e., molecules having chemically different structures. A compound library is screenable, that is, the compound library members therein may be subject to screening assays.

By “members of the compound library” it is meant those compounds within a compound library that may be screened for a preselected attribute. Any type of molecule that is capable of potentially having the preselected attribute may be a member of a compound library. “Screening assays” are selective assays designed to identify, isolate, and/or determine the structure of, compounds within the library that have a preselected, typically desirable, attribute.

Libraries of candidate ligands are assayed by the fluorescence polarization assay. Libraries may consist of chemically synthesized peptides; peptidomimetics; and arrays of combinatorial chemicals that are large or small, focused or nonfocused. By “focused” it is meant that the collection of compounds is prepared using the structure of previously characterized ligands and/or pharmacophores

Compound libraries may contain molecules isolated from natural sources, artificially synthesized molecules, or molecules synthesized, isolated, or otherwise prepared in such a manner so as to have one or more moieties variable, e.g., moieties that are independently isolated or randomly synthesized. Types of molecules in compound libraries include but are not limited to organic compounds, polypeptides and nucleic acids as those terms are used herein, and derivatives, conjugates and mixtures thereof.

Compound libraries of the invention may be prepared or obtained by any means including, but not limited to, combinatorial chemistry techniques, fermentation methods, plant and cellular extraction procedures and the like (see, e.g., Cwirla et al., Biochemistry 1990, 87, 6378-6382; Houghten et al., Nature 1991, 354, 84-86; Lam et al., Nature 1991, 354, 82-84; Brenner et al., Proc. Natl. Acad. Sci. USA 1992, 89, 5381-5383; R. A. Houghten, Trends Genet. 1993, 9, 235-239; E. R. Felder, Chimia 1994, 48, 512-541; Gallop et al., J. Med. Chem. 1994, 37, 1233-1251; Gordon et al., J. Med. Chem. 1994, 37, 1385-1401; Carell et al., Chem. Biol. 1995, 3, 171-183; Madden et al., Perspectives in Drug Discovery and Design 2, 269-282; Lebl et al., Biopolymers 1995, 37 177-198); small molecules assembled around a shared molecular scaffold; collections of chemicals that have been assembled by various commercial and noncommercial groups, natural products; extracts of marine organisms, fungi, bacteria, and plants.

Preferred libraries are prepared in a homogenous reaction mixture, and separation of unreacted reagents from members of the library is not required prior to screening. Although many combinatorial chemistry approaches are based on solid State chemistry, liquid phase combinatorial chemistry is capable of generating libraries (Sun C M., Recent advances in liquid-phase combinatorial chemistry, Combinatorial Chemistry & High Throughput Screening. 2:299-318, 1999). Preferred libraries are prepared in a homogenous reaction mixture, i.e., one in which separation of unreacted reagents from members of the library is not required prior to screening.

Derivatives and Conjugates

A “derivative” of a compound (the “parent compound”) is a chemically modified form of that compound. Parent compounds can be members of a compound mixture or library, candidate compounds, lead compounds, pharmacophores and drugs. Preferred derivatives are those that retain one or more desirable attributes or activities, including biological activities, of the parent compound. More preferred derivatives are those that are enhanced with regard to one or more desirable properties or activities of the parent compound. Most preferred derivatives are those that have been enhanced with regard to one or more desirable properties or activities of the parent compound, in order to maximize (or minimize) a preselected desirable (or undesirable) property or activity in order to optimize a preselected attribute of the derivative. As one non-limiting example, sFv-5AF is a derivative of sFv-5A. Examples of derivatives for various types of ligands are given below in the description of each type of ligand. Because it is a polypeptide, derivatives of pIgR include proteins, fusion proteins, oligopeptides and polypeptide derivatives as these terms are described herein.

Conjugate molecules are also within the scope of the invention. A “conjugate molecule” or “conjugate” is a chemical compound or complex comprising two or more independently preparable molecules (i.e., each may be independently synthesized, isolated or otherwise prepared) in tight association with each other. Non-limiting examples of tight associations are strong intramolecular surface interactions (e.g., avidin-biotin; GST-glutathione; nucleic acid base-pairing; and the like) and chemical bonds. The association may be stable, that is, resistant to degradative processes and conditions such as those that occur during the manufacture, formulation, storage and administration of conjugate molecules. The association may be labile. A conjugate can be designed to separate into various components in certain conditions. The term conjugate thus encompasses conjugates, and separable components thereof, that separate into two or more components in vivo (e.g., prodrugs). The term conjugate also encompasses conjugates, and associable components thereof, that result from the in vivo assembly of molecules into a conjugate.

By “conjugating” it is meant to place two or molecules in tight association with each other. Conjugate molecules consist essentially of a first moiety that is a pIgR ligand and a second moiety that is biologically active, wherein preferred conjugates retains, to an effective degree, the biological activity of the second moiety; that is, preferred conjugate are biologically active. Preferred conjugates of the invention comprise one or more pIgR ligands and one or more other type of moiety (i.e., a moiety that is not a pIgR-targeting element) that are biologically active. Preferred conjugates retain the functionality of the pIgR ligand or the biological activity of the other moiety, and most preferred conjugates retain the functionality of the pIgR ligand and the biological activity of the second moiety.

Conjugates are classified herein as simple, mixed or complex conjugates. The term “simple conjugate” refers to a conjugate of a small molecule, nucleic acid, or polypeptide with another molecule of the same type (i.e., another small molecule, nucleic acid, or polypeptide, respectively). A non-limiting example of a simple conjugate is a pair of small molecules that have been chemically bonded to each other. Another non-limiting example of a simple conjugate is a nucleic acid molecule in which nucleotide sequences from two or more different nucleic acids have been combined into one nucleic acid via, e.g., chemical synthesis of a nucleic acid product having sequences derived from two or more nucleic acids; directed polymerase chain reaction (PCR) of two or more nucleic acid substrates using primers designed to generate a single nucleic acid as a product of the PCR; ligation of or recombination between two or more nucleic acids that generates a recombinant nucleic acid as a product; optionally followed in each instance by biological (cloning) or chemical (PCR) amplification of the product. A fusion protein, which comprises two or more polypeptide chains from different sources and which may be produced via recombinant DNA technology, is another non-limiting example of a simple conjugate. Because the other molecule(s) of the conjugate may have identical structures, the conjugate may be a multimeric form of a single molecular species (e.g., a dimer protein comprising two identical polypeptide molecules).

The term “mixed conjugate” refers to a conjugate comprising two different types of molecules. Non-limiting examples of mixed conjugates according to the invention include a conjugate of one type of targeting element (e.g., a pIgR ligand) with one or more other molecules of a different type. Non-limiting examples include a small molecule pIgR ligand conjugated to a polypeptide or a nucleic acid; a polypeptide pIgR ligand (e.g., sFv-5A and derivatives thereof) conjugated to a nucleic acid or small molecule; a nucleic acid pIgR ligand conjugated to a small molecule or polypeptide pIgR ligand, etc. Complex conjugates comprise three or more different types of molecules.

Compositions and methods for conjugating pIgR ligands are described in U.S. patent application Ser. Nos. 60/248,478 and 60/248,819 (attorney docket Nos. 030854.0009.PRV2 and 030854.0009.PRV3 entitled “Protein Conjugates of pIgR Ligands for the Delivery of Therapeutic and Diagnostic Proteins” by Houston, L. L., and Hawley, Stephen), filed Nov. 13, 2000 and Nov. 14, 2000 respectively, which is hereby incorporated by reference.

Screening Assays and Compound Optimization

“Screening assays” are selective assays designed to identify, isolate, and/or determine the structure of, compounds within the library that have a preselected, typically desirable, attribute. Non-limiting examples of screening assays for isolating ligands to pIgR and the pIgR stalk molecule are described herein. Any apparatus for a screening that is appropriate for a particular selective assay or type of compound library may be used. See, for example, U.S. Pat. No. 6,054,047.

By “identify a compound” it is meant to measure a detectable signal produced by compounds in the library that have the preselected attribute. By “isolate a compound” it is meant to prepare a composition that is enriched for candidate compounds, or that comprises partially purified or purified candidate compounds. By “determine of the structure of a compound” it is meant to determine all, or a functional portion of, a compound or set of chemically or functionally related compounds. Such determinations include but are not limited to determining a compound's (i) antigenicity and cross-reactivity with other compounds; (ii) chemical formula; (iii) chemical structure; (iv) in the case of polypeptides and nucleic acids, the amino acid or nucleotide sequence; (v) primary, secondary and/or tertiary structure; (vi) three-dimensional structure; (vii) in the case of compounds that associate with the same or other compounds, the number of compound molecules present in the multimeric or multi-protein complex formed from such association, and/or structure at the compound's interface with other molecules in a complex; and (viii) structure in a conjugate, particularly in circumstances wherein the portion of the conjugate that is not a screenable compound has the same structure of, or serves the same purpose as, other members of the compound library.

Members of the compound library are screened for a preselected attribute in a first screening assay. As determined by the first screening assay, compounds having the preselected attribute have been identified, isolated, and/or have had their structure determined, and are “candidate compounds.” Candidate compounds, conjugates and/or derivatives thereof are subject to reiterative rounds of the same and/or additional screening assays in order to identify, isolate and/or determine the structure of “lead compounds,” i.e., candidate compounds having the highest amount of the preselected attribute, wherein the number of lead compounds is greater than the number of candidate compounds.

The structures of lead compounds, or conjugates and derivatives thereof, are determined and/or approximated in vitro or in silico; and the biological activities of these compounds are measured in vivo and/or in vitro. Such information is used in rational drug design in order to develop structurally related molecules or moieties having the desired biological activity. See, e.g., Padlan et al., Methods Enz. 203:3-21, 1991; Bolger et al., Methods Enz. 203:21-45, 1991; Perez, Methods Enz. 203:510-556, 1991; Martin, Methods Enz. 203:587-613, 1991; Neidle et al., Methods Enz. 203:433-458, 1991; Loechler, Methods Enz. 203:458-476, 1991).

As used herein, the term “pharmacophore” refers to a particular three-dimensional arrangement of chemical moieties that are required for a compound to have a desired attribute. When a pharmacophore is derived from a ligand for a target molecule, it comprises an atom or groups of atoms, and of the associating forces (e.g., hydrogen bonding, van der Waals interactions, etc.) that form the interface between the ligand and the target. Pharmacophores are used, e.g., to search 3-dimensional databases to identify known molecules, or used in molecular modeling to predict the structure of novel molecules having a 3-dimensional arrangement like that of the phramacophore. For example, a pharmacophore derived from a ligand is used to identify or predict arrangements of atoms that will fit into the same “pocket” of the target molecule as the ligand. Pharmacophores are used to identify and predict molecular frameworks that approximate the shape of the phramacophore. These frameworks can be used as the basis to prepare a library, pool or mixture of different, but structurally related, compounds. Such collections are called focused, smart or pharmacophore libraries, pools and mixtures. To the extent that a model of a given pharmacophore is accurate, such collections are enriched for molecules having at least one desirable attribute or activity, and, if so, screening assays of such collections will yield a higher percentage of candidate and lead compounds.

As used herein, “drugs” are compound obtainable via the invention that (i) are (and may have been derivitized, conjugated, modeled or otherwise optimized to be) compounds that have an effective amount of a desirable attribute, which may be a biological activity, (ii) preferably compounds that have (and may have been derivitized, conjugated, modeled or otherwise optimized to have) a minimal amount, or no detectable amount, of one or more undesirable attributes, that (iii) may be formulated into pharmaceutical compositions capable of delivering an effective dose of the drug to an animal in need thereof. A compound that is “capable of delivering an effective dose of the drug” may achieve this effect directly (i.e., the compound is a drug) or indirectly (e.g., the compound is a prodrug that is converted into a drug in vivo). Thus, prodrugs based on the compounds described herein are included in the scope of the invention.

For the compounds of the invention, desirable attributes include but are not limited to binding to a target, which may be a pIgR stalk molecule, stability in vitro or in vivo, ability to be formulated into a preselected pharmaceutical composition, and the ability to undergo endocytosis, transcytosis, intracellular localization or exocytosis. Undesirable attributes include but are not limited to undesirable side effects, instability in vivo or in vitro, little or no ability to be formulated into a desired pharmaceutical composition, and lack of specificity for the target molecule.

If a compound library is screenable for binding to pIgR or a portion thereof, the candidate compounds are candidate pIgR ligands. Lead compounds prepared therefrom, or from structural information thereof, may be derivatives or conjugates of candidate pIgR ligands.

High Throughput Screening (HTS)

Screening assays may be low throughput screening assays, wherein no more than a mixture of compounds is run through a selective assay at one time in one or more iterations of the assay. In high throughput screening (HTS) assays, compounds are screened en masse; i.e., a large collection (at least a pool, preferably a library) of compounds are rapidly run through one or more selective assays, typically with the assistance of automated and semi-automated devices, especially library preparation devices and compound detection devices. Various methods for analyzing the interaction of a known or suspected ligand molecule with its preselected target are known. Such methods include without limitation assays that detect bound or unbound ligands, and/or unbound or bound target molecules. Such assays may utilize mass spectrometry, phosphorescence, chemiluminescence, luminescence, fluorescence polarization, resonance energy transfer, liquid chromatography; assays based on affinity capture and/or competitive inhibition of known or suspected ligands directed to a target molecule; assays that determine binding to the site (e.g., epitope) or measure the degree (e.g., Kd, Ki, etc.) of binding of known or suspected ligands for target molecules; and scintillation proximity assays. In most assays, at least one detectably labeled molecular reagent is used.

HTS techniques, devices and software are described in the following publications, which are incorporated herein by reference: Strege M A, High-performance liquid chromatographic-electrospray ionization mass spectrometric analyses for the integration of natural products with modern high-throughput screening, Journal of Chromatography. B, Biomedical Sciences & Applications. 725:67-78, 1999; Grabley S. Thiericke R., Bioactive agents from natural sources: trends in discovery and application, Advances in Biochemical Engineering-Biotechnology. 64:101-54, 1999; Kenny B A. Bushfield M. Parry-Smith D J. Fogarty S. Treherne J M., The application of high-throughput screening to novel lead discovery, Progress in Drug Research. 51:245-69, 1998; Rodrigues A D., Preclinical drug metabolism in the age of high-throughput screening: an industrial perspective, Pharmaceutical Research. 14:1504-10, 1997; Humphery-Smith I. Cordwell S J. Blackstock W P., Proteome research: complementarity and limitations with respect to the RNA and DNA worlds, Electrophoresis 18:1217-42, 1997.

Kd may be measured in solution using techniques and compositions described in the following publications. Blake, D. A.; Blake, R. C.; Khosraviani, M.; Pavlov, A. R. “Immunoassays for Metal Ions.” Analytica Chimica Acta 1998, 376, 13-19. Blake, D. A.; Chakrabarti, P.; Khosraviani, M.; Hatcher, F. M.; Westhoff, C. M.; Goebel, P.; Wylie, D. E.; Blake, R. C. “Metal Binding Properties of a Monoclonal Antibody Directed toward Metal-Chelate Complexes.” Journal of Biological Chemistry 1996, 271(44), 27677-27685. Blake, D. A.; Khosraviani, M.; Pavlov, A. R.; Blake, R. C. “Characterization of a Metal-Specific Monoclonal Antibody.” Aga, D. S.; Thurman, E. M., Eds.; ACS Symposium Series 657; American Chemical Society: Washington, DC, 1997; pp 49-60.

Kd is measured using immobilized binding components on a chip, for example, on a BIAcore chip using surface plasmon resonance. Surface plasmon resonance is used to characterize the microscopic association and dissociation constants of reaction between sFv directed against pIgG associated molecules and pIgR and pIgR fragments. Such general methods are described in the following references and are incorporated herein by reference (Vely F. Trautmann A. Vivier E., BIAcore analysis to test phosphopeptide-SH2 domain interactions, Methods in Molecular Biology. 121:313-21, 2000; Liparoto S F. Ciardelli T L., Biosensor analysis of the interleukin-2 receptor complex, Journal of Molecular Recognition. 12:316-21, 1999; Lipschultz C A. Li Y. Smith-Gill S., Experimental design for analysis of complex kinetics using surface plasmon resonance, Methods. 20):310-8, 2000; Malmqvist M., BIACORE: an affinity biosensor system for characterization of biomolecular interactions, Biochemical Society Transactions. 27:335-40, 1999; Alfthan K., Surface plasmon resonance biosensors as a tool in antibody engineering, Biosensors & Bioelectronics. 13:653-63, 1998; Fivash M. Towler E M. Fisher R J., BIAcore for macromolecular interaction, Current Opinion in Biotechnology. 9:97-101, 1998; Price M R. Rye P D. Petrakou E. Murray A. Brady K. Imai S. Haga S. Kiyozuka Y. Schol D. Meulenbroek M F. Snijdewint F G. Von Mensdorff-Pouilly S. Verstraeten R A. Kenemans P. Blockzjil A. Nilsson K. Nilsson O. Reddish M. Suresh M R. Koganty R R. Fortier S. Baronic L. Berg A. Longenecker M B. Hilgers J. et al.; Summary report on the ISOBM TD-4 Workshop: analysis of 56 monoclonal antibodies against the MUC1 mucin. San Diego, Calif., Nov. 17-23, 1996, Tumour Biology. 19 Suppl 1:1-20, 1998; Malmqvist M. Karlsson R, Biomolecular interaction analysis: affinity biosensor technologies for functional analysis of proteins, Current Opinion in Chemical Biology. 1:378-83, 1997; O'Shannessy D J. Winzor D J., Interpretation of deviations from pseudo-first-order kinetic behavior in the characterization of ligand binding by biosensor technology, Analytical Biochemistry. 236:275-83, 1996; Malmborg A C. Borrebaeck C A, BIAcore as a tool in antibody engineering, Journal of Immunological Methods. 183:7-13, 1995; Van Regenmortel M H., Use of biosensors to characterize recombinant proteins, Developments in Biological Standardization. 83:143-51, 1994; O'Shannessy D J., Determination of kinetic rate and equilibrium binding constants for macromolecular interactions: a critique of the surface plasmon resonance literature, Current Opinion in Biotechnology. 5:65-71, 1994).

III. Small Molecules & Derivatives

The term “small molecule” includes any chemical or other moiety that can act to affect biological processes. Small molecules can include any number of therapeutic agents presently known and used, or can be small molecules synthesized in a library of such molecules for the purpose of screening for biological function(s). Small molecules are distinguished from macromolecules by size. The small molecules of this invention usually have molecular weight less than about 5,000 daltons (Da), preferably less than about 2,500 Da, more preferably less than 1,000 Da, most preferably less than about 500 Da.

Small molecules include without limitation organic compounds, peptidomimetics and conjugates thereof. As used herein, the term “organic compound” refers to any carbon-based compound other than macromolecules such nucleic acids and polypeptides. In addition to carbon, organic compounds may contain calcium, chlorine, fluorine, copper, hydrogen, iron, potassium, nitrogen, oxygen, sulfur and other elements. An organic compound may be in an aromatic or aliphatic form. Non-limiting examples of organic compounds include acetones, alcohols, anilines, carbohydrates, monosaccharides, oligosaccharides, polysaccharides, amino acids, nucleosides, nucleotides, lipids, retinoids, steroids, proteoglycans, ketones, aldehydes, saturated, unsaturated and polyunsaturated fats, oils and waxes, alkenes, esters, ethers, thiols, sulfides, cyclic compounds, heterocylcic compounds, imidizoles and phenols. An organic compound as used herein also includes nitrated organic compounds and halogenated (e.g., chlorinated) organic compounds. Methods for preparing peptidomimetics are described below. Collections of small molecules, and small molecules identified according to the invention are characterized by techniques such as accelerator mass spectrometry (AMS; see Turteltaub et al., Curr Pharm Des 2000 6(10):991-1007, Bioanalytical applications of accelerator mass spectrometry for pharmaceutical research; and Enjalbal et al., Mass Spectrom Rev 2000 19(3):139-61, Mass spectrometry in combinatorial chemistry.)

Preferred small molecules are relatively easier and less expensively manufactured, formulated or otherwise prepared. Preferred small molecules are stable under a variety of storage conditions. Preferred small molecules may be placed in tight association with macromolecules to form molecules that are biologically active and that have improved pharmaceutical properties. Improved pharmaceutical properties include changes in circulation time, distribution, metabolism, modification, excretion, secretion, elimination, and stability that are favorable to the desired biological activity. Improved pharmaceutical properties include changes in the toxicological and efficacy characteristics of the chemical entity.

IV. Nucleic Acids

Traditionally, techniques for detecting and purifying target molecules have used polypeptides, such as antibodies, that specifically bind such targets. Nucleic acids have long been known to specifically bind other nucleic acids (e.g., ones having complementary sequences). Recently, however, nucleic acids that bind non-nucleic target molecules have been described. See, e.g., Blackwell, T. K., et al., Science (1990) 250:1104-1110; Blackwell, T. K., et al., Science (1990) 250:1149-1152; Tuerk, C., and Gold, L., Science (1990) 249:505-510; Joyce, G. F., Gene (1989) 82:83-87.

As applied to aptamers, the term “binding” specifically excludes the “Watson-Crick”-type binding interactions (i.e., A:T and G:C base-pairing) traditionally associated with the DNA double helix. The term “aptamer” thus refers to a nucleic acid or a nucleic acid derivative that specifically binds to a target molecule, wherein the target molecule is either (i) not a nucleic acid, or (ii) a nucleic acid or structural element thereof that is bound through mechanisms other than duplex- or triplex-type base pairing. Such a molecule is called a “non-nucleic molecule” herein.

Structures of Aptamers

“Nucleic acids,” as used herein, refers to nucleic acids that are isolated a natural source; prepared in vitro, using techniques such as PCR amplification or chemical synthesis; prepared in vivo, e.g., via recombinant DNA technology; or by any appropriate method. Nucleic acids may be of any shape (linear, circular, etc.) or topology (single-stranded, double-stranded, supercoiled, etc.). The term “nucleic acids” also includes without limitation nucleic acid derivatives such as peptide nucleic acids (PNA's) and polypeptide-nucleic acid conjugates; nucleic acids having at least one chemically modified sugar residue, backbone, internucleotide linkage, base, nucleoside, or nucleotide analog; as well as nucleic acids having chemically modified 5′ or 3′ ends; and nucleic acids having two or more of such modifications. Not all linkages in a nucleic acid need to be identical.

Nucleic acids that are aptamers are often, but need not be, prepared as oligonucleotides. Oligonucleotides include without limitation RNA, DNA and mixed RNA-DNA molecules having sequences of lengths that have minimum lengths of 2, 4, 6, 8, 10, 11, 12, 13, 14 or 15 nucleotides, and maximum lengths of about 100, 75, 50, 40, 25, 20 or 15 or more nucleotides, irrespectively. In general, a minimum of approximately 6 nucleotides, preferably 10, and more preferably 14 or 15 nucleotides, is necessary to effect specific binding.

In general, the oligonucleotides may be single-stranded (ss) or double-stranded (ds) DNA or RNA, or conjugates (e.g., RNA molecules having 5′ and 3′ DNA “clamps”) or hybrids (e.g., RNA:DNA paired molecules), or derivatives (chemically modified forms thereof). However, single-stranded DNA is preferred, as DNA is less susceptible to nuclease degradation than RNA. Similarly, chemical modifications that enhance an aptamer's specificity or stability are preferred.

Chemical modifications that may be incorporated into aptamers include with neither limitation nor exclusivity base modifications, sugar modifications, and backbone modifications.

Base modifications: The base residues in aptamers may be other than naturally occurring bases (e.g., A, G, C, T, U, 5MC, and the like). Derivatives of purines and pyrimidines are known in the art; an exemplary but not exhaustive list includes aziridinylcytosine, 4-acetylcytosine, 5-fluorouracil, 5-bromouracil, 5-carboxymethylaminomethyl-2-thiouracil, 5-carboxymethylaminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine (5MC), N6-methyladenine, 7-methylguanine, 5-methylaminomethyluracil, 5-methoxyaminomethyl-2-thiouracil, beta-D-mannosylqueosine, 5-methoxyuracil, 2-methylthio-N-6-isopentenyladenine, uracil-5-oxyacetic acid methylester, pseudouracil, queosine, 2-thiocytosine, 5-methyl-2-thiouracil, 2-thiouracil, 4-thiouracil, 5-methyluracil, uracil-5-oxyacetic acid, and 2,6-diaminopurine. In addition to nucleic acids that incorporate one or more of such base derivatives, nucleic acids having nucleotide residues that are devoid of a purine or a pyrimidine base may also be included in aptamers.

Sugar modifications: The sugar residues in aptamers may be other than conventional ribose and deoxyribose residues. By way of non-limiting example, substitution at the 2′-position of the furanose residue enhances nuclease stability. An exemplary, but not exhaustive list, of modified sugar residues includes 2′ substituted sugars such as 2′-O-methyl-, 2′-O-alkyl, 2′-O-allyl, 2′-S-alkyl, 2′-S-allyl, 2′-fluoro-, 2′-halo, or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and abasic nucleoside analogs such as methyl riboside, ethyl riboside or propylriboside.

Backbone modifications: Chemically modified backbones include, by way of non-limiting example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Chemically modified backbones that do not contain a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages, including without limitation morpholino linkages; siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; and amide backbones.

Preparation and Identification of Aptamers

In general, techniques for identifying aptamers involve incubating a preselected non-nucleic target molecule with mixtures (2 to 50 members), pools (50 to 5,000 members) or libraries (50 or more members) of different nucleic acids that are potential aptamers under conditions that allow complexes of target molecules and aptamers to form. By “different nucleic acids” it is meant that the nucleotide sequence of each potential aptamer may be different from that of any other member, that is, the sequences of the potential aptamers are random with respect to each other. Randomness can be introduced in a variety of manners such as, e.g., mutagenesis, which can be carried out in vivo by exposing cells harboring a nucleic acid with mutagenic. agents, in vitro by chemical treatment of a nucleic acid, or in vitro by biochemical replication (e.g., PCR) that is deliberately allowed to proceed under conditions that reduce fidelity of replication process; randomized chemical synthesis, i.e., by synthesizing a plurality of nucleic acids having a preselected sequence that, with regards to at least one position in the sequence, is random. By “random at a position in a preselected sequence” it is meant that a position in a sequence that is normally synthesized as, e.g., as close to 100% A as possible (e.g., 5′-C-T-T-A-G-T-3′) is allowed to be randomly synthesized at that position (C-T-T-N-G-T, wherein N indicates a randomized position where, for example, the synthesizing reaction contains 25% each of A,T,C and G; or x % A, w % T, y % C and z % G, wherein x+w+y+z=100. In later stages of the process, the sequences are increasingly less randomized and consensus sequences may appear; in any event, it is preferred to ultimately obtain an aptamer having a unique nucleotide sequence.

Aptamers and pools of aptamers are prepared, identified, characterized and/or purified by any appropriate technique, including those utilizing in vitro synthesis, recombinant DNA techniques, PCR amplification, and the like. After their formation, target:aptamer complexes are then separated from the uncomplexed members of the nucleic acid mixture, and the nucleic acids that can be prepared from the complexes are candidate aptamers (at early stages of the technique, the aptamers generally being a population of a multiplicity of nucleotide sequences having varying degrees of specificity for the target). The resulting aptamer (mixture or pool) is then substituted for the starting apatamer (library or pool) in repeated iterations of this series of steps. When a limited number (e.g., a pool or mixture, preferably a mixture with less than 10 members, most preferably 1) of nucleic acids having satisfactory specificity is obtained, the aptamer is sequenced and characterized. Pure preparations of a given aptamer are generated by any appropriate technique (e.g., PCR amplification, in vitro chemical synthesis, and the like).

For example, Tuerk and Gold (Science (1990) 249:505-510) describe the use of a procedure termed “systematic evolution of ligands by exponential enrichment” (SELEX). In this method, pools of nucleic acid molecules that are randomized at specific positions are subjected to selection for binding to a nucleic acid-binding protein (see, e.g., PCT International Publication No. WO 91/19813 and U.S. Pat. No. 5,270,163). The oligonucleotides so obtained are sequenced and otherwise characterization. Kinzler, K. W., et al. (Nucleic Acids Res. (1989) 17:3645-3653) used a similar technique to identify synthetic double-stranded DNA molecules that are specifically bound by DNA-binding polypeptides. Ellington, A. D., et al. (Nature (1990) 346: 818-822) describe the production of a large number of random sequence RNA molecules and the selection and identification of those that bind specifically to specific dyes such as Cibacron blue.

Another technique for identifying nucleic acids that bind non-nucleic target molecules is the oligonucleotide combinatorial technique described by Ecker, D. J. et al. (Nuc. Acids Res. 21, 1853 (1993)) known as “synthetic unrandomization of randomized fragments” (SURF), which is based on repetitive synthesis and screening of increasingly simplified sets of oligonucleotide analogue libraries, pools and mixtures (Tuerk, C. and Gold, L. (Science 249, 505 (1990)). The starting library consists of oligonucleotide analogues of defined length with one position in each pool containing a known analogue and the remaining positions containing equimolar mixtures of all other analogues. With each round of synthesis and selection, the identity of at least one position of the oligomer is determined until the sequences of optimized nucleic acid ligand aptamers are discovered.

Once a particular candidate aptamer has been identified through a SURF, SELEX or any other technique, its nucleotide sequence can be determined (as is known in the art), and its three-dimensional molecular structure can be examined by nuclear magnetic resonance (NMR). These techniques are explained in relation to the determination of the three-dimensional structure of a nucleic acid ligand that binds thrombin in Padmanabhan, K. et al., J. Biol. Chem. 24, 17651 (1993); Wang, K. Y. et al., Biochemistry 32, 1899 (1993); and Macaya, R. F. et al., Proc. Nat'l. Acad. Sci. USA 90, 3745 (1993). Selected aptamers may be resynthesized using one or more modified bases, sugars or backbone linkages. Aptamers consist essentially of the minimum sequence of nucleic acid needed to confer binding specificity, but may be extended on the 5′ end, the 3′ end, or both, or may be otherwise derivatized or conjugated.

V. Polypeptides & Derivatives

As used herein, the term “polypeptide” includes proteins, fusion proteins, oligopeptides and polypeptide derivatives, with the exception that peptidomnimetics are considered to be small molecules herein. Although they are polypeptides, antibodies and their derivatives are described in a separate section. Antibodies and antibody derivatives are described in a separate section, but antibodies and antibody derivatives are, for purposes of the invention, treated as a subclass of the polypeptides and derivatives.

A “protein” is a molecule having a sequence of amino acids that are linked to each other in a linear molecule by peptide bonds. The term protein refers to a polypeptide that is isolated from a natural source, or produced from an isolated cDNA using recombinant DNA technology; and has a sequence of amino acids having a length of at least about 200 amino acids.

A “fusion protein” is a type of recombinant protein that has an amino acid sequence that results from the linkage of the amino acid sequences of two or more normally separate polypeptides. Methods of preparing and using fusion proteins are described in U.S. patent application Ser. No. 60/237,929 (attorney docket No. 030854.0009 entitled “Genetic Fusions of pIgR Ligands and Biologically Active Polypeptides for the Delivery of Therapeutic and Diagnostic Proteins” by Houston, L. L., Glynn, Jacqueline M., and Sheridan, Philip L.), filed Oct. 2, 2000, which is incorporated in its entirety herein.

A “protein fragment” is a proteolytic fragment of a larger polypeptide, which may be a protein or a fusion protein. A proteolytic fragment may be prepared by in vivo or in vitro proteolytic cleavage of a larger polypeptide, and is generally too large to be prepared by chemical synthesis. Proteolytic fragments have amino acid sequences having a length from about 200 to about 1,000 amino acids.

An “oligopeptide” is a polypeptide having a short amino acid sequence (i.e., 2 to about 200 amino acids). An oligopeptide is generally prepared by chemical synthesis.

Although oligopeptides and protein fragments may be otherwise prepared, it is possible to use recombinant DNA technology and/or in vitro biochemical manipulations. For example, a nucleic acid encoding an amino acid sequence may be prepared and used as a template for in vitro transcription/translation reactions. In such reactions, an exogenous nucleic acid encoding a preselected polypeptide is introduced into a mixture that is essentially depleted of exogenous nucleic acids that contains all of the cellular components required for transcription and translation. One or more radiolabeled amino acids are added before or with the exogenous DNA, and transcription and translation are allowed to proceed. Because the only nucleic acid present in the reaction mix is the exogenous nucleic acid added to the reaction, only polypeptides encoded thereby are produced, and incorporate the radiolabelled amino acid(s). In this manner, polypeptides encoded by a preselected exogenous nucleic acid are radiolabeled. Although other proteins are present in the reaction mix, the preselected polypeptide is the only one that is produced in the presence of the radiolabeled amino acids and is thus uniquely labeled.

As is explained in detail below, “polypeptide derivatives” include without limitation mutant polypeptides, chemically modified polypeptides, and peptidominetics.

The polypeptides of this invention, including the analogs and other modified variants, may generally be prepared following known techniques. Preferably, synthetic production of the polypeptide of the invention may be according to the solid phase synthetic method. For example, the solid phase synthesis is well understood and is a common method for preparation of polypeptides, as are a variety of modifications of that technique [Merrifield (1964), J. Am. Chem. Soc., 85: 2149; Stewart and Young (1984), Solid Phase polypeptide Synthesis, Pierce Chemical Company, Rockford, Ill.; Bodansky and Bodanszky (1984), The Practice of polypeptide Synthesis, Springer-Verlag, N.Y.; Atherton and Sheppard (1989), Solid Phase polypeptide Synthesis: A Practical Approach, IRL Press, New York]. See, also, the specific method described in Example 1 below.

Alternatively, polypeptides of this invention may be prepared in recombinant systems using polynucleotide sequences encoding the polypeptides. For example, fusion proteins are typically prepared using recombinant DNA technology.

Polypeptide Derivatives

A “derivative” of a polypeptide is a compound that is not, by definition, a polypeptide, i.e., it contains at least one chemical linkage that is not a peptide bond. Thus, polypeptide derivatives include without limitation proteins that naturally undergo post-translational modifications such as, e.g., glycosylation. It is understood that a polypeptide of the invention may contain more than one of the following modifications within the same polypeptide. Preferred polypeptide derivatives retain a desirable attribute, which may be biological activity; more preferably, a polypeptide derivative is enhanced with regard to one or more desirable attributes, or has one or more desirable attributes not found in the parent polypeptide. Although they are described in this section, peptidomimetics are taken as small molecules in the present disclosure.

A. Mutant Polypeptides

A polypeptide having an amino acid sequence identical to that found in a protein prepared from a natural source is a “wildtype” polypeptide. Mutant oligopeptides can be prepared by chemical synthesis, including without limitation combinatorial synthesis.

Mutant polypeptides larger than oligopeptides can be prepared using recombinant DNA technology by altering the nucleotide sequence of a nucleic acid encoding a polypeptide. Although some alterations in the nucleotide sequence will not alter the amino acid sequence of the polypeptide encoded thereby (“silent” mutations), many will result in a polypeptide having an altered amino acid sequence that is altered relative to the parent sequence. Such altered amino acid sequences may comprise substitutions, deletions and additions of amino acids, with the proviso that such amino acids are naturally occurring amino acids.

Thus, subjecting a nucleic acid that encodes a polypeptide to mutagenesis is one technique that can be used to prepare mutant polypeptides, particularly ones having substitutions of amino acids but no deletions or insertions thereof. A variety of mutagenic techniques are known that can be used in vitro or in vivo including without limitation chemical mutagenesis and PCR-mediated mutagenesis. Such mutagenesis may be randomly targeted (i.e., mutations may occur anywhere within the nucleic acid) or directed to a section of the nucleic acid that encodes a stretch of amino acids of particular interest. Using such techniques, it is possible to prepare randomized, combinatorial or focused compound libraries, pools and mixtures.

Polypeptides having deletions or insertions of naturally occurring amino acids may be synthetic oligopeptides that result from the chemical synthesis of amino acid sequences that are based on the amino acid sequence of a parent polypeptide but which have one or more amino acids inserted or deleted relative to the sequence of the parent polypeptide. Insertions and deletions of amino acid residues in polypeptides having longer amino acid sequences may be prepared by directed mutagenesis.

B. Chemically Modified Polypeptides

As contemplated by this invention, the term “polypeptide” includes those having one or more chemical modification relative to another polypeptide, i.e., chemically modified polypeptides. The polypeptide from which a chemically modified polypeptide is derived may be a wildtype protein, a mutant protein or a mutant polypeptide, or polypeptide fragments thereof; an antibody or other polypeptide ligand according to the invention including without limitation single-chain antibodies, bacterial proteins and polypeptide derivatives thereof; or polypeptide ligands prepared according to the disclosure. Preferably, the chemical modification(s) confer(s) or improve(s) desirable attributes of the polypeptide but does not substantially alter or compromise the biological activity thereof. Desirable attributes include but are limited to increased shelf-life; enhanced serum or other in vivo stability; resistance to proteases; and the like. Such modifications include by way of non-limiting example N-terminal acetylation, glycosylation, and biotinylation.

Polypeptides with N-Terminal or C-Terminal Chemical Groups

An effective approach to confer resistance to peptidases acting on the N-terminal or C-terminal residues of a polypeptide is to add chemical groups at the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum (Powell et al. (1993), Pharma. Res. 10: 1268-1273). Other chemical modifications which enhance serum stability include, but are not limited to, the addition of an N-terminal alkyl group, consisting of a lower alkyl of from 1 to 20 carbons, such as an acetyl group, and/or the addition of a C-terminal amide or substituted amide group.

Polypeptides with a Terminal D-Amino Acid

The presence of an N-terminal D-amino acid increases the serum stability of a polypeptide that otherwise contains L-amino acids, because exopeptidases acting on the N-terminal residue cannot utilize a D-amino acid as a substrate. Similarly, the presence of a C-terminal D-amino acid also stabilizes a polypeptide, because serum exopeptidases acting on the C-terminal residue cannot utilize a D-amino acid as a substrate. With the exception of these terminal modifications, the amino acid sequences of polypeptides with N-terminal and/or C-terminal D-amino acids are usually identical to the sequences of the parent L-amino acid polypeptide.

Polypeptides with Substitution of Natural Amino Acids by Unnatural Amino Acids

Substitution of unnatural amino acids for natural amino acids in a subsequence of a polypeptide can confer or enhance desirable attributes including biological activity. Such a substitution can, for example, confer resistance to proteolysis by exopeptidases acting on the N-terminus. The synthesis of polypeptides with unnatural amino acids is routine and known in the art (see, for example, Coller, et al. (1993), cited above).

Post-Translational Chemical Modifications

Different host cells will contain different post-translational modification mechanisms that may provide particular types of post-translational modification of a fusion protein if the amino acid sequences required for such modifications is present in the fusion protein. A large number (˜100) of post-translational modifications have been described, a few of which are discussed herein. One skilled in the art will be able to choose appropriate host cells, and design chimeric genes that encode protein members comprising the amino acid sequence needed for a particular type of modification.

Glycosylation is one type of post-translational chemical modification that occurs in many eukaryotic systems, and may influence the activity, stability, pharmacogenetics, immunogenicity and/or antigenicity of proteins. However, specific amino acids must be present at such sites to recruit the appropriate glycosylation machinery, and not all host cells have the appropriate molecular machinery. Saccharomyces cerevisieae and Pichia pastoris provide for the production of glycosylated proteins, as do expression systems that utilize insect cells, although the, pattern of glyscoylation may vary depending on which host cells are used to produce the fusion protein.

Another type of post-translation modification is the phosphorylation of a free hydroxyl group of the side chain of one or more Ser, Thr or Tyr residues. Protein kinases catalyze such reactions. Phosphorylation is often reversible due to the action of a protein phosphatase, an enzyme that catalyzes the dephosphorylation of amino acid residues.

Differences in the chemical structure of amino terminal residues result from different host cells, each of which may have a different chemical version of the methionine residue encoded by a start codon, and these will result in amino termnini with different chemical modifications.

For example, many or most bacterial proteins are synthesized with an amino terminal amino acid that is a modified form of methionine, i.e, N-formyl-methionine (fMet). Although the statement-is often made that all bacterial proteins are synthesized with an fmet initiator amino acid; although this may be true for E. coli, recent studies have shown that it is not true in the case of other bacteria such as Pseudomonas aeruginosa (Newton et al., J. Biol. Chem. 274:22143-22146, 1999). In any event, in E. coli, the formyl group of fMet is usually enzymatically removed after translation to yield an amino terminal methionine residue, although the entire fMet residue is sometimes removed (see Hershey, Chapter 40, “Protein Synthesis” in: Escherichia Coli and Salmonella Typhimurium: Cellular and Molecular Biology, Neidhardt, Frederick C., Editor in Chief, American Society for Microbiology, Washington, D.C., 1987, Volume 1, pages 613-647, and references cited therein.) E. coli mutants that lack the enzymes (such as, e.g., formylase) that catalyze such post-translational modifications will produce proteins having an amino terminal fMet residue (Guillon et al., J. Bacteriol. 174:4294-4301, 1992).

In eukaryotes, acetylation of the initiator methionine residue, or the penultimate residue if the initiator methionine has been removed, typically occurs co- or post-translationally. The acetylation reactions are catalyzed by N-terminal acetyltransferases (NATs, a.k.a. N-alpha-acetyltransferases), whereas removal of the initiator methionine residue is catalyzed by methionine aminopeptidases (for reviews, see Bradshaw et al., Trends Biochem. Sci. 23:263-267, 1998; and Driessen et al., CRC Crit. Rev. Biochem. 18:281-325, 1985). Amino terminally acetylated proteins are said to be “N-acetylated,” “N alpha acetylated” or simply “acetylated.”

Another post-translational process that occurs in eukaryotes is the alpha-amidation of the carboxy terminus. For reviews, see Eipper et al. Annu. Rev. Physiol. 50:333-344, 1988, and Bradbury et al. Lung Cancer 14:239-251, 1996. About 50% of known endocrine and neuroendocrine peptide hormones are alpha-amidated (Treston et al., Cell Growth Differ. 4:911-920, 1993). In most cases, carboxy alpha-amidation is required to activate these peptide hormones.

C. Peptidomimetics

In general, a polypeptide mimetic (“peptidomimetic”) is a molecule that mimics the biological activity of a polypeptide but is no longer peptidic in chemical nature. By strict definition, a peptidomimetic is a molecule that contains no peptide bonds (that is, amide bonds between amino acids). However, the term peptidomimetic is sometimes used to describe molecules that are no longer completely peptidic in nature, such as pseudo-peptides, semi-peptides and peptoids. Examples of some peptidomimetics by the broader definition (where part of a polypeptide is replaced by a structure lacking peptide bonds) are described below. Whether completely or partially non-peptide, peptidomimetics according to this invention provide a spatial arrangement of reactive chemical moieties that closely resembles the three-imensional arrangement of active groups in the polypeptide on which the peptidomimetic is based. As a result of this similar active-site geometry, the peptidorimetic has effects on biological systems that are similar to the biological activity of the polypeptide.

There are several potential advantages for using a mimetic of a given polypeptide rather than the polypeptide itself. For example, polypeptides may exhibit two undesirable attributes, i.e., poor bioavailability and short duration of action. Peptidomimetics are often small enough to be both orally active and to have a long duration of action. There are also problems associated with stability, storage and immunoreactivity for polypeptides that are not experienced with peptidomimetics.

Candidate, lead and other polypeptides having a desired biological activity can be used in the development of peptidomimetics with similar biological activities. Techniques of developing peptidomimetics from polypeptides are known. Peptide bonds can be replaced by non-peptide bonds that allow the peptidomimetic to adopt a similar structure, and therefore biological activity, to the original polypeptide. Further modifications can also be made by replacing chemical groups of the amino acids with other chemical groups of similar structure. The development of peptidomnimetics can be aided by determining the tertiary structure of the original polypeptide, either free or bound to a ligand, by NMR spectroscopy, crystallography and/or computer-aided molecular modeling. These techniques aid in the development of novel compositions of higher potency and/or greater bioavailability and/or greater stability than the original polypeptide (Dean (1994), BioEssays, 16: 683-687; Cohen and Shatzmiller (1993), J. Mol. Graph., 11: 166-173; Wiley and Rich (1993), Med. Res. Rev., 13: 327-384; Moore (1994), Trends Pharmacol. Sci., 15: 124-129; Hruby (1993), Biopolymers, 33: 1073-1082; Bugg et al. (1993); Sci. Am., 269: 92-98, all incorporated herein by reference].

Thus, through use of the methods described above, the present invention provides compounds exhibiting enhanced therapeutic activity in comparison to the polypeptides described above. The peptidomrimetic compounds obtained by the above methods, having the biological activity of the above named polypeptides and similar three-dimensional structure, are encompassed by this invention. It will be readily apparent to one skilled in the art that a peptidomimetic can be generated from any of the modified polypeptides described in the previous section or from a polypeptide bearing more than one of the modifications described from the previous section. It will furthermore be apparent that the peptidomrnimetics of this invention can be further used for the development of even more potent non-peptidic compounds, in addition to their utility as therapeutic compounds.

Specific examples of peptidomirnetics derived from the polypeptides described in the previous section are presented below. These examples are illustrative and not limiting in terms of the other or additional modifications.

Peptides with a Reduced Isostere Pseudopeptide Bond

Proteases act on peptide bonds. It therefore follows that substitution of peptide bonds by pseudopeptide bonds confers resistance to proteolysis. A number of pseudopeptide bonds have been described that in general do not affect polypeptide structure and biological activity. The reduced isostere pseudopeptide bond is a suitable pseudopeptide bond that is known to enhance stability to enzymatic cleavage with no or little loss of biological activity (Couder, et al. (1993), Int. J. Polypeptide Protein Res. 41:181-184, incorporated herein by reference). Thus, the amino acid sequences of these compounds may be identical to the sequences of their parent L-amino acid polypeptides, except that one or more of the peptide bonds are replaced by an isostere pseudopeptide bond. Preferably the most N-terminal peptide bond is substituted, since such a substitution would confer resistance to proteolysis by exopeptidases acting on the N-terminus.

Peptides with a Retro-Inverso Pseudopeptide Bond

To confer resistance to proteolysis, peptide bonds may also be substituted by retro-inverso pseudopeptide bonds (Dalpozzo, et al. (1993), Int. J. Polypeptide Protein Res. 41:561-566, incorporated herein by reference). According to this modification, the amino acid sequences of the compounds may be identical to the sequences of their L-amino acid parent polypeptides, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus.

Peptoid Derivatives

Peptoid derivatives of polypeptides represent another form of modified polypeptides that retain the important structural determinants for biological activity, yet eliminate the peptide bonds, thereby conferring resistance to proteolysis (Simon, et al., 1992, Proc. Natl. Acad. Sci. USA, 89:9367-9371 and incorporated herein by reference). Peptoids are oligomers of N-substituted glycines. A number of N-alkyl groups have been described, each corresponding to the side chain of a natural amino acid.

VI. Antibodies and Antibody Derivatives, Including Single Chain Antibodies

The term “antibody” is meant to encompass an immunoglobulin molecule obtained by in vitro or in vivo generation of an immunogenic response, and includes both polyclonal, monospecific and monoclonal antibodies. An “immunogenic response” is one that results in the production of antibodies directed to one or more proteins after the appropriate cells have been contacted with such proteins, or polypeptide derivatives thereof, in a manner such that one or more portions of the protein function as epitopes. An epitope is a single antigenic determinant in a molecule. In proteins, particularly denatured proteins, an epitope is typically defined and represented by a contiguous amino acid sequence. However, in the case of nondenatured proteins, epitopes also include structures, such as active sites, that are formed by the three-dimensional folding of a protein in a manner such that amino acids from separate portions of the amino acid sequence of the protein are brought into close physical contact with each other.

Wildtype antibodies have four polypeptide chains, two identical heavy chains and two identical light chains. Both types of polypeptide chains have constant regions, which do not vary or vary minimally among antibodies of the same class (i.e, IgA, IgM, etc.), and variable regions. As is explained below, variable regions are unique to a particular antibody and comprise a recognition element for an epitope.

Each light chain of an antibody is associated with one heavy chain, and the two chains are linked by a disulfide bridge formed between cysteine residues in the carboxy-terminal region of each chain, which is distal from the amino terminal region of each chain that constitutes its portion of the antigen binding domain. Antibody molecules are further stabilized by disulfide bridges between the two heavy chains in an area known as the hinge region, at locations nearer the carboxy terminus of the heavy chains than the locations where the disulfide bridges between the heavy and light chains are made. The hinge region also provides flexibility for the antigen-binding portions of an antibody.

An antibody's specificity is determined by the variable regions located in the amino terminal regions of the light and heavy chains. The variable regions of a light chain and associated heavy chain form an “antigen binding domain” that recognizes a specific epitope; an antibody thus has two antigen binding domains. The antigen binding domains in a wildtype antibody are directed to the same epitope of an immunogenic protein, and a single wildtype antibody is thus capable of binding two molecules of the immunogenic protein at the same time.

Compositions of antibodies have, depending on the manner in which they are prepared, different types of antibodies. Types of antibodies of particular interest include polyclonal, monospecific and monoclonal antibodies.

Polyclonal antibodies are generated in a immunogenic response to a protein having many epitopes. A composition of polyclonal antibodies thus includes a variety of different antibodies directed to the same and to different epitopes within the protein. Methods for producing polyclonal antibodies are known in the art (see, e.g., Cooper et al., Section. III of Chapter 11 in: Short Protocols in Molecular Biology, 2nd Ed., Ausubel et al., eds., John Wiley and Sons, New York, 1992, pages 11-37 to 11-41).

Monospecific antibodies (a.k.a. antipeptide antibodies) are generated in a humoral response to a short (typically, 5 to 20 amino acids) immunogenic polypeptide that corresponds to a few (preferably one) isolated epitopes of the protein from which it is derived. A plurality of monospecific antibodies includes a variety of different antibodies directed to a specific portion of the protein, i.e, to an amino acid sequence that contains at least one, preferably only one, epitope. Methods for producing monospecific antibodies are known in the art (see, e.g., Cooper et al., Section III of Chapter 11 in: Short Protocols in Molecular Biology, 2nd Ed., Ausubel et al., eds., John Wiley and Sons, New York, 1992, pages 11-42 to 11-46).

A monoclonal antibody is a specific antibody that recognizes a single specific epitope of an immunogenic protein. In a plurality of a monoclonal antibody, each antibody molecule is identical to the others in the plurality. In order to isolate a monoclonal antibody, a clonal cell line that expresses, displays and/or secretes a particular monoclonal antibody is first identified; this clonal cell line can be used in one method of producing the antibodies of the invention. Methods for the preparation of clonal cell lines and of monoclonal antibodies expressed thereby are known in the art (see, for example, Fuller et al., Section II of Chapter 11 in: Short Protocols in Molecular Biologyi 2nd Ed., Ausubel et al., eds., John Wiley and Sons, New York, 1992, pages 11-22 to 11-11-36).

Variants and derivatives of antibodies include antibody and T-cell receptor fragments that retain the ability to specifically bind to antigenic determinants. Preferred fragments include Fab fragments (i.e, an antibody fragment that contains the antigen-binding domain and comprises a light chain and part of a heavy chain bridged by a disulfide bond); Fab′ (an antibody fragment containing a single anti-binding domain comprising an Fab and an additional portion of the heavy chain through the hinge region); F(ab′)2 (two Fab′ molecules joined by interchain disulfide bonds in the hinge regions of the heavy chains; the Fab′ molecules may be directed toward the same or different epitopes); a bispecific Fab (an Fab molecule having two antigen binding domains, each of which may be directed to a different epitope); a single chain Fab chain comprising a variable region, a.k.a., a sFv (the variable, antigen-binding determinative region of a single light and heavy chain of an antibody linked together by a chain of 10-25 amino acids); a disulfide-linked Fv, or dsFv (the variable, antigen-binding determinative region of a single light and heavy chain of an antibody linked together by a disulfide bond); a camelized VH (the variable, antigen-binding determinative region of a single heavy chain of an antibody in which some amino acids at the VH interface are those found in the heavy chain of naturally occurring camel antibodies); a bispecific sFv (a sFv or a dsFv molecule having two antigen-binding domains, each of which may be directed to a different epitope); a diabody (a dimerized sFv formed when the VH domain of a first sFv assembles with the VL domain of a second sFv and the VL domain of the first sFv assembles with the VH domain of the second sFv; the two antigen-binding regions of the diabody may be directed towards the same or different epitopes); and a triabody (a trimerized sFv, formed in a manner similar to a diabody, but in which three antigen-binding domains are created in a single complex; the three antigen binding domains may be directed towards the same or different epitopes). Derivatives of antibodies also include one or more CDR sequences of an antibody combining site. The CDR sequences may be linked together on a scaffold when two or more CDR sequences are present.

The term “antibody” also includes genetically engineered antibodies and/or antibodies produced by recombinant DNA techniques and “humanized” antibodies. Humanized antibodies have been modified, by genetic manipulation and/or in vitro treatment to be more human, in terms of amino acid sequence, glycosylation pattern, etc., in order to reduce the antigenicity of the antibody or antibody fragment in an animal to which the antibody is intended to be administered (Gussow et al., Methods Enz. 203:99-121, 1991).

Methods of Preparing Antibodies and Antibody Variants

The antibodies and antibody fragments of the invention may be produced by any suitable method, for example, in vivo (in the case of polyclonal and monospecific antibodies), in cell culture (as is typically the case for monoclonal antibodies, wherein hybridoma cells expressing the desired antibody are cultured under appropriate conditions), in in vitro translation reactions, and in recombinant DNA expression systems (the latter method of producing proteins is described in more detail herein in the section entitled “Methods of Producing Fusion Proteins”). Antibodies and antibody variants can be produced from a variety of animal cells, preferably from mammalian cells, with murine and human cells being particularly preferred. Antibodies that include non-naturally occurring antibody and T-cell receptor variants that retain only the desired antigen targeting capability conferred by an antigen binding site(s) of an antibody can be produced by known cell culture techniques and recombinant DNA expression systems (see, e.g., Johnson et al., Methods in Enzymol. 203:88-98, 1991; Molloy et al., Mol. Immunol. 32:73-81, 1998; Schodin et al., J. Immunol. Methods 200:69-77, 1997). Recombinant DNA expression systems are typically used in the production of antibody variants such as, e.g., bispecific antibodies and sFv molecules. Preferred recombinant DNA expression systems include those that utilize host cells and expression constructs that have been engineered to produce high levels of a particular protein. Preferred host cells and expression constructs include Escherichia coli; harboring expression constructs derived from plasmids or viruses (bacteriophage); yeast such as Sacharomyces cerevisieae or Fichia pastoras harboring episomal or chromosomally integrated expression constructs; insect cells and viruses such as Sf 9 cells and baculovirus; and mammalian cells harboring episomal or chromosomally integrated (e.g., retroviral) expression constructs (for a review, see Verma et al., J. Immunol. Methods 216:165-181, 1998). Antibodies can also be produced in plants (U.S. Pat. No. 6,046,037; Ma et al., Science 268:716-719, 1995) or by phage display technology (Winter et al., Annu. Rev. Immunol. 12:433-455, 1994).

XenoMouse strains are genetically engineered mice in which the murine IgH and Igk locci have been functionally replaced by their Ig counterparts on yeast artificial YAC transgenes. These human Ig transgenes can carry the majority of the human variable repertoire and can undergo class switching from IgM to IgG isotypes. The immune system of the xenomouse recognizes administered human antigens as foreign and produces a strong humoral response. The use of XenoMouse in conjunction with well-established hybridomas techniques, results in fully human IgG mAbs with sub-nanomolar affinities for human antigens (for a review, see Green, J. Immunol. Methods 231:11-23, 1999).

The single-chain antibody sFv-5A, and derivatives thereof such as sFv-5AF, is used in competition assays to identify molecules that prevent the binding of sFv-5A to this region of the polyimmunoglobulin receptor (pIgR) and/or the pIgR stalk protein, or derivatives or conjugates thereof.

The sFv-5A compound is a non-limiting example of a sFv (or antibodies and fragments derived therefrom) that may be used as a pIgR targeting element in compounds that are intended to undergo endocytosis, transcytosis and/or exocytosis across an epithelial surface. Any sFv that reacts with the pIgR stalk molecule or transcytotic molecule is used to deliver macromolecules into, through and out of cell, especially epithelial cells, but some may have better binding and transport features than others.

Anti-pIgR sFvs are characterized with respect to their epitope binding sites. However, some sFv molecules may not react with pIgR in such a way as to identify a linear epitope, because the eptiope that is recognized may be comprised of regions of amino acid sequence that are remote in the linear sequence of pIgR. Reduced reactivity or no reactivity at all with a nest set of oligopeptides is usually achieved with ‘non-linear’ or ‘conformational’ epitopes.

These and other sFvs are tagged with antigenic peptides, such as myc and flag, to which commercially available antibodies and antibody-conjugates are available. The tag sequence is placed on the amino terminus or the carboxy terminus of the sFv and is separated by a flexible tether, such as (Gly4Ser)x, where x is 1 to 4 and preferably 2 to 3. Tagged sFv is quantitated by reaction with antibody-conjugates that are detectable. Detection is achieved by using horse radish peroxidase or alkaline phosphate or other detectable systems attached to the antibody that has specificity for the tag.

VII. Characterization and Optimization of Compounds

Molecular attributes of compounds that are or comprise a ligand of pIgR, including but not limited to candidate compounds, lead compounds, pharmacophores and drugs, as well as derivatives and conjugates thereof, are characterized and optimized according to techniques appropriate for the type of molecule in question.

Molecular attributes of particular interest include but are nor limited to parameters associated with ligand:pIgR binding; antigericity; histocompatibility; protease resistance; stability in serum or other bodily fluids or portions ex vivo, in vivo or in pharmaceutical compositions; half-life in vitro (e.g., in a pharmaceutical composition), ex vivo or in vivo; and the like. Such characterization may, but need not, be carried out in the course of preparing (e.g., identifying, isolating, synthesizing, derivativizing, purifying or optimizing) pIgR ligands, and derivatives and conjugates thereof.

Binding Parameters. As explained in detail supra, parameters associated with binding of a ligand and its target molecule, such as Kd or Ki, may be determined or estimated according to techniques and formulae known in the art.

Serum Stability. A compound is detectably labeled, or is capable of binding a detectably labeled reagent, and predetermined amounts of the compound are incubated in sera over a period of time, and samples are taken at regular intervals. The rate of decrease of signal is inversely proportional to the serum stability of the compound. The half-life of the compound in serum is calculated according to formulae known in the art.

Epitope Determination of Antibodies and Antibody Derivatives. The specific site of a molecule that binds an antibody or antibody derivative is an “epitope.” As is described herein, the amino acid sequences of epitopes for a single-chain antibody, sFv-5A, were determined by measuring the immunoreactivity of a nested set of oligopeptides, each about 15 amino acids long, that included amino acid sequences found in the pIgR stalk (see Example 2). Computer predictions of the location of epitopes in polypeptides based on their primary structures can be used to help determine the sequence of polypeptide epitopes (see, e.g., Pellequer et al., Methods Enz. 203:176-201, 1991).

Direct binding of a IgR ligand is detected by several methods including those using biotinylated and radiolabelled small molecules. Selection using peptides displayed on filamentous phage is also effective in isolating small molecules that can be refined by methods known to those skilled in the art. Such methods include molecular modelling and refinement and chemical modifications of candidate and lead compounds and pharmacophores.

Once a pharmacophore has shown to bind pIgR, that structure is refined and improved in its binding characteristics. For example, peptidomimetics are designed from conformational studies of peptides that may have reduced conformational flexibility, such as those with cystine loops in their structure. Small focused libraries are produced that capture the essential parts of the pharmacophore and add or subtract moieties therefrom. Analysis of each of these derivatives in quantitative assays leads to a data set of quantitative information that relate the structure of the compounds to their ability to bind to pIgR. Specificity and selectivity information when carefully analyzed will lead to improved candidates with better selectivity, specificity, and binding characteristics.

Pharmacophores that bind specifically and selectively with pIgR are conjugated to target drugs that are to be delivered across epithelial barriers. The pharmacophores are designed so that linkages between the pharmacophore and the target drug is achieved. The linkages are either highly stable (strong covalent bonds) or unstable (disulfide, ester, peptide, or noncovalent bonds). Examples of highly stable bonds include thiol-maleimide conjugates, hindered disulfides where the carbons adjacent to the disulfide bond have methyl groups attached, some ester bonds, some peptide bonds, ethers, etc. Examples of unstable bonds include disulfides, ester bonds that are subject to attack by esterases, peptide bonds that are subject to attack by proteases and peptidases, ester bonds that are unstable to the lower pH in the endosomes that are encountered during transcytosis, peptide bonds that are acted upon by proteases in the endosomes, disulfides that are reduced in the endosomes, etc.

In general, small pharmacophores will be less likely to produce an immune response, but will have a much shorter half-life in blood than larger pharmacophores. Preferred pharmacophores are metabolized to nontoxic and nonimmunogenic compounds.

If a pharmacophore is a polypeptide, it may be incorporated into a fusion protein that is intended to be used as a drug. Sites within the target molecule may also be identified in which to insert a pharmacophore. For example, a loop in a three dimensional structure is modified and extended to incorporate the pharmacophore without altering the pharmacological function of the target drug in unacceptable ways. A loop is identified on the surface of the target drug that if extended would not interfere with its binding properties to its normal ligand. Modification of this loop by inserting a peptide sequence is possible if the peptide insertion has the ability to assume a conformation that returns it to the surface of the target drug in such a way that the overall conformation of the target drug is retained.

VIII. Pharmaceutical Compositions and Therapeutic Methods

Another aspect of the invention is drawn to compositions, including but not limited to pharmaceutical compositions. According to the invention, a “composition” refers to a mixture comprising at least one carrier, preferably a physiologically acceptable carrier, and one or more pIgR-targeting protein conjugates. The term “carrier” defines a chemical compound that does not inhibit or prevent the incorporation of the biologically active peptide(s) into cells or tissues. A carrier typically is an inert substance that allows an active ingredient to be formulated or compounded into a suitable dosage form (e.g., a pill, a capsule, a gel, a film, a tablet, a microparticle (e.g., a microsphere), a solution; an ointment; a paste, an aerosol, a droplet, a colloid or an emulsion etc.). A “physiologically acceptable carrier” is a carrier suitable for use under physiological conditions that does not abrogate (reduce, inhibit, or prevent) the biological activity and properties of the compound. For example, dimethyl sulfoxide (DMSO) is a carrier as it facilitates the uptake of many organic compounds into the cells or tissues of an organism. Preferably, the carrier is a physiologically acceptable carrier, preferably a pharmaceutically or veterinarily acceptable carrier, in which the chimeric pIgR-targeting protein is disposed.

A “pharmaceutical composition” refers to a composition wherein the carrier is a pharmaceutically acceptable carrier, while a “veterinary composition” is one wherein the carrier is a veterinarily acceptable carrier. The term “pharmaceutically acceptable carrier” or “veterinarily acceptable carrier” includes any medium or material that is not biologically or otherwise undesirable, i.e, the carrier may be administered to an organism along with a chimeric pIgR-targeting protein conjugate, composition or compound without causing any undesirable biological effects or interacting in a deleterious manner with the complex or any of its components or the organism. Examples of pharmaceutically acceptable reagents are provided in The United States Pharmacopeia, The National Formulary, United States Pharmacopeial Convention, Inc., Rockville, Md. 1990, hereby incorporated by reference herein into the present application. The terms “therapeutically effective amount” or “pharmaceutically effective amount” mean an amount sufficient to induce or effectuate a measurable response in the target cell, tissue, or body of an organism. What constitutes a therapeutically effective amount will depend on a variety of factors which the knowledgeable practitioner will take into account in arriving at the desired dosage regimen.

The compositions of the invention can further comprise other chemical components, such as diluents and excipients. A “diluent” is a chemical compound diluted in a solvent, preferably an aqueous solvent, that facilitates dissolution of the chimeric pIgR-targeting protein in the solvent, and it may also serve to stabilize the biologically active form of the chimeric pIgR-targeting protein or one or more of its components. Salts dissolved in buffered solutions are utilized as diluents in the art. For example, preferred diluents are buffered solutions containing one or more different salts. A preferred buffered solution is phosphate buffered saline (particularly in conjunction with compositions intended for pharmaceutical administration), as it mimics the salt conditions of human blood. Since buffer salts can control the pH of a solution at low concentrations, a buffered diluent rarely modifies the biological activity of a biologically active peptide.

An “excipient” is any more or less inert substance that can be added to a composition in order to confer a suitable property, for example, a suitable consistency or to form a drug. Suitable excipients and carriers include, in particular, fillers such as sugars, including lactose, sucrose, mannitol, or sorbitol cellulose preparations such as, for example, maize starch, wheat starch, rice starch, agar, pectin, xanthan gum, guar gum, locust bean gum, hyaluronic acid, casein potato starch, gelatin, gum tragacanth, polyacrylate, methyl cellulose, hydroxypropylmethyl-cellulose, sodium carboxymethylcellulose, and/or polyvinylpyrrolidone (PVP). If desired, disintegrating agents can also be included, such as cross-linked polyvinyl pyrrolidone, agar, or alginic acid or a salt thereof such as sodium alginate. Other suitable excipients and carriers include hydrogels, gellable hydrocolloids, and chitosan. Chitosan microspheres and microcapsules can be used as carriers. See WO 98/52547 (which describes microsphere formulations for targeting compounds to the stomach, the formulations comprising an inner core (optionally including a gelled hydrocolloid) containing one or more active ingredients, a membrane comprised of a water insoluble polymer (e.g., ethylcellulose) to control the release rate of the active ingredient(s), and an outer layer comprised of a bioadhesive cationic polymer, for example, a cationic polysaccharide, a cationic protein, and/or a synthetic cationic polymer; U.S. Pat. No. 4,895,724. Typically, chitosan is cross-linked using a suitable agent, for example, glutaraldehyde, glyoxal, epichlorohydrin, and succinaldehyde. Compositions employing chitosan as a carrier can be formulated into a variety of dosage forms, including pills, tablets, microparticles, and microspheres, including those providing for controlled release of the active ingredient(s). Other suitable bioadhesive cationic polymers include acidic gelatin, polygalactosamine, polyamino acids such as polylysine, polyhistidine, polyornithine, polyquaternary compounds, prolamine, polyimine, diethylaminoethyldextran (DEAE), DEAE-imine, DEAE-methacrylate, DEAE-acrylamide, DEAE-dextran, DEAE-cellulose, poly-p-aminostyrene, polyoxethane, copolymethacrylates, polyamidoarnines, cationic starches, polyvinylpyridine, and polythiodiethylaminomethylethylene.

The compositions of the invention can be formulated in any suitable manner. The chimeric pIgR-targeting proteins therein may be uniformly (homogeneously) or non-uniformly (heterogenously) dispersed in the carrier. Suitable formulations include dry and liquid formulations. Dry formulations include freeze dried and lyophilized powders, which are particularly well suited for aerosol delivery to the sinuses or lung, or for long term storage followed by reconstitution in a suitable diluent prior to administration. Other preferred dry formulations include those wherein a composition according to the invention is compressed into tablet or pill form suitable for oral administration or compounded into a sustained release formulation. When the composition is intended for oral administration but the chimeric pIgR-targeting protein is to be delivered to epithelium in the intestines, it is preferred that the formulation be encapsulated with an enteric coating to protect the formulation and prevent premature release of the chimeric pIgR-targeting proteins included therein. As those in the art will appreciate, the compositions of the invention can be placed into any suitable dosage form. Pills and tablets represent some of such dosage forms. The compositions can also be encapsulated into any suitable capsule or other coating material, for example, by compression, dipping, pan coating, spray drying, etc. Suitable capsules include those made from gelatin and starch. In turn, such capsules can be coated with one or more additional materials, for example, and enteric coating, if desired. Liquid formulations include aqueous formulations, gels, and emulsions.

Some preferred embodiments concern compositions that comprise a bioadhesive, preferably a mucoadhesive, coating. A “bioadhesive coating” is a coating that allows a substance (e.g., a composition or chimeric pIgR-targeting protein according to the invention) to adhere to a biological surface or substance better than occurs absent the coating. A “mucoadhesive coating” is a preferred bioadhesive coating that allows a substance, for example, a composition according to the invention, to adhere better to mucosa occurs absent the coating. For example, micronized particles (e.g., particles having a mean diameter of about 5, 10, 25, 50, or 100 μm) can be coated with a mucoadhesive. The coated particles can then be assembled into a dosage form suitable for delivery to an organism. Preferably, and depending upon the location where the cell surface transport moiety to be targeted is expressed, the dosage form is then coated with another coating to protect the formulation until it reaches the desired location, where the mucoaclesive enables the formulation to be retained while the chimeric pIgR-targeting proteins interact with the target cell surface transport moiety.

The compositions of the invention facilitate administration of chimeric pIgR-targeting proteins to an organism, preferably an animal, preferably a mammal, bird, fish, insect, or arachnid. Preferred mammals include bovine, canine, equine, feline, ovine, and porcine animals, and non-human primates. Humans are particularly preferred. Multiple techniques of administering or delivering a compound exist in the art including, but not limited to, oral, rectal (e.g. an enema or suppository) aerosol (e.g., for nasal or pulmonary delivery), parenteral, and topical administration. Preferably, sufficient quantities of the biologically active peptide are delivered to achieve the intended effect. The particular amount of biologically active peptide to be delivered will depend on many factors, including the effect to be achieved, the type of organism to which the composition is delivered, delivery route, dosage regimen, and the age, health, and sex of the organism. As such, the particular dosage of chimeric pIgR-targeting protein included in a given formulation is left to the ordinarily skilled artisan's discretion.

Those skilled in the art will appreciate that when the compositions of the present invention are administered as agents to achieve a particular desired biological result, which may include a therapeutic or protective effect(s) (including vaccination), it may be necessary to combine the fusion proteins of the invention with a suitable pharmaceutical carrier. The choice of pharmaceutical carrier and the preparation of the fusion protein as a therapeutic or protective agent will depend on the intended use and mode of administration. Suitable formulations and methods of administration of therapeutic agents include those for oral, pulmonary, nasal, buccal, ocular, dermal, rectal, or vaginal delivery.

Depending on the mode of delivery employed, the context-dependent functional entity can be delivered in a variety of pharmaceutically acceptable forms. For example, the context-dependent functional entity can be delivered in the form of a solid, solution, emulsion, dispersion, micelle, liposome, and the like, incorporated into a pill, capsule, tablet, suppository, areosol, droplet, or spray. Pills, tablets, suppositories, areosols, powders, droplets, and sprays may have complex, multilayer structures and have a large range of sizes. Aerosols, powders, droplets, and sprays may range from small (1 micron) to large (200 micron) in size.

Pharmaceutical compositions of the present invention can be used in the form of a solid, a lyophilized powder, a solution, an emulsion, a dispersion, a micelle, a liposome, and the like, wherein the resulting composition contains one or more of the compounds of the present invention, as an active ingredient, in admixture with an organic or inorganic carrier or excipient suitable for enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The carriers which can be used include glucose, lactose, mannose, gum acacia, gelatin, mannitol, starch paste, magnesium trisilicate, talc, corn starch, keratin, colloidal silica, potato starch, urea, medium chain length triglycerides, dextrans,and other carriers suitable for use in manufacturing preparations, in solid, semisolid, or liquid form. In addition auxiliary, stabilizing, thickening and coloring agents and perfumes may be used. Examples of a stabilizing dry agent includes triulose, preferably at concentrations of 0.1% or greater (See, e.g., U.S. Pat. No. 5,314,695).

The active compound (i.e, protein conjugate) is included in the pharmaceutical composition in an amount sufficient to produce the desired effect upon the process or condition of diseases.

Uses of Ligands

The invention's compositions facilitate administration of pIgR-targeting protein conjugates to an organism, preferably an animal, preferably a mammal, bird, fish, insect, or arachnid. Preferred mammals include bovine, canine, equine, feline, ovine, and porcine animals, and non-human primates. Humans are particularly preferred. Multiple techniques of administering or delivering a compound exist in the art including, but not limited to, oral, aerosol (e.g., for nasal or pulmonary delivery), parenteral, and topical administration. Preferably, sufficient quantities of the biologically active peptide are delivered to achieve the intended effect. The particular amount of biologically active peptide to be delivered will depend on many factors, including the effect to be achieved, the type of organism to which the composition is delivered, delivery route, dosage regimen, and the age, health, and sex of the organism. As such, the particular dosage of pIgR-targeting protein conjugate included in a given formulation is left to the ordinarily skilled artisan's discretion.

Thus, another aspect of the invention relates to delivering a composition according to the invention to an organism. As a result, a pIgR-targeting protein conjugate of the composition is delivered to cells expressing the pIgR protein. The pIgR expressing cell of the present invention is preferably a mammalian cell and more preferably a mammalian epithelial cell that normally secretes IgA. Such epithelial cells that normally secrete IgA can be found in the intestinal tract, the oral cavity, the nasal cavity, the respiratory tract, the ocular surfaces, and the dermal surfaces of a mammal.

A related aspect concerns various applications for the pIgR-targeting protein conjugates and compositions of the invention. These include prophylactic and therapeutic applications. For example, a preferred prophylactic application is vaccination, wherein a chimeric pIgR-targeting protein or composition according to the invention allows an antigen including a super antigen, presented as the biologically active peptide, to be delivered and elicit an immune response, preferably a protective immune response, in the organism to which the composition was administered. In general, the vaccines of the invention are administered in a manner compatible with the dosage formulation, and in such amount as will be therapeutically effective and immunogenic. The quantity to be administered depends on the subject to be treated, capacity of the subject's immune system to synthesize antibodies, and the degree of protection desired. Precise amounts of active ingredient required to be administered depend on the judgment of the practitioner and are peculiar to each individual. However, suitable dosage ranges are of the order of several hundred micrograms active ingredient per individual. Suitable regimes for initial administration and booster shots are also variable, but are typified by an initial administration followed in one or two week intervals by a subsequent injection or other administration.

In another therapeutic context, the pIgR-targeting protein conjugates and compositions allow a biologically active peptide having a therapeutic effect to be efficaciously delivered as part of a pIgR-targeting protein conjugate. Because pIgR-targeting protein conjugates are delivered into cells by active transport, the instant compositions afford better control over bioavailability of biologically active peptides, as compared to passive transport mechanisms. As such, the pIgR-targeting protein conjugates and compositions of the invention enable improved uptake and utilization of the biologically active peptide.

The invention's compositions facilitate administration of pIgR-targeting protein conjugates to an organism, preferably an animal, preferably a mammal, bird, fish, insect, or arachnid. Preferred mammals include bovine, canine, equine, feline, ovine, and porcine animals, and non-human primates. Humans are particularly preferred. Multiple techniques of administering or delivering a compound exist in the art including, but not limited to, oral, aerosol (e.g., for nasal or pulmonary delivery), parenteral, and topical administration. Preferably, sufficient quantities of the biologically active peptide are delivered to achieve the intended effect. The particular amount of biologically active peptide to be delivered will depend on many factors, including the effect to be achieved, the type of organism to which the composition is delivered, delivery route, dosage regimen, and the age, health, and sex of the organism. As such, the particular dosage of pIgR-targeting protein conjugate included in a given formulation is left to the ordinarily skilled artisan's discretion.

The protein conjugates of the invention are also useful in diagnostic and related applications. Another aspect of the invention is compositions and methods comprising the protein conjugates of the invention for the diagnosis and monitoring of certain diseases and disorders, preferably in kit form. This aspect is useful for assaying and monitoring the course of the diagnosis and prognosis of disease, for monitoring the effectiveness and/or distribution of a therapeutic agent or an endogenous protein, in a patient as well as other related functions.

In this aspect of the invention, it may be desirable to monitor or determine if, or dtermine the degree to which, a patient's pIgR-displaying cells are capable of, or presently are, endocytosing a detectably labeled fusion protein of the invention. Such methods are used in a variety of systems depending on the nature of the pIgR-targeting element(s) of a given protein conjugate.

For example, the degree to which a patient, or a biological sample therefrom, endocytoses a protein conjugate that has a pIgR-targeting element derived from a bacterial protein that binds pIgR is a measure of a patient's susceptibility to infection by bacteria having that element. A higher degree or rate of uptake of the detectably labeled protein conjugate indicates that the patient is more susceptible to such infection.

As another example, the activity, distribution and/or concentration of endogenous pIgR proteins may be altered in various ways during the course of a disease or disorder. The pIgR proteins in a patient are measured over the course of a disease for diagnostic and prognostic purposes, as well as over the course of treatment of a disease or disorder, in order to monitor the effects on pIgR proteins. Diseases to which this aspect of the invention can be applied include but are not limited to diseases that involve the respiratory system, such as lung cancer and tumors, asthma, pathogenic infections, allergy-related disorders, and the like; the gastrointestinal tract, including cancers, tumors, pathogenic infections, disorders relating to gastroinstestinal hormones, Chron's disease, eating disorders, and the like; and any disease or disorder that is known or suspected to involve pIgR-displaying cells.

Protein conjugates may be detectably labeled by virute of comprising a detectable polypeptide such as, e.g., a green fluorescent protein (GFP) or a derivative thereof. If the protein conjugate comprises an epitope for which antibodies are available (including but nor limited to commercially avalable ones such as c-myc epitope and the FLAG-tag), it may be detected using any of a variety of immunoassays such as enzyme-linked immunosorbent assay (ELISA) or a radioimmunoassay (RIA).

The contents of the articles, patent, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications or other documents.

### EXAMPLES

### Example 1

### Nucleotide Sequences of a Simian pIgR

1.1. Isolation of pIgR cDNA from Monkey Intestinal Tissue

Rhesus and Cynomolgus monkey intestinal tissue was obtained from Yerkes Regional Primate Center (Atlanta, Ga.). At least 30 grams of tissue specimens were each prepared from ileum and colon sections where the tissue was excised within one-half hour postmortem, rinsed free of feces with PBS, and then rapidly frozen using liquid nitrogen, shipped overnight on dry ice and stored frozen at −80° C.

A section of cynomolgus colon weighing 5.3 grams (wet weight) was placed in a 50 ml conical tube and rapidly washed 3-5 times with approximately a 30 ml volume of PBS to remove residual fecal material. The colon segment was removed to a very small plastic weigh boat and a longitudinal incision was made exposing the luminal surface, which was quickly and gently rinsed with ˜50 mls of PBS. One (1) ml of TRIzol reagent (Life Technologies) was layered and massaged on the luminal surface, collected in a 15 ml conical tube, and total cellular RNA isolated as per manufacturer's instructions. Briefly, the RNA solution was centrifuged at 12,000×g to remove insoluble cellular debri, and 700 uls of total solution transferred to an microfuge tube. 140 uls of chloroform was added the solution centrifuged at 14,000 rpms for 15 minutes at 4° C. 430 uls of aqueous phase was collected, 215 uls of isopropanol added, incubated at room temperature for 10 minutes, and the RNA precipitated by centrifugation at 14,000 rpms for 10 minutes at 4° C. The white pellet was washed with 1 ml of 75% ethanol, air dried for 5-10 minutes, and the RNA pellet resuspended in 50 uls of DEPC-treated water. Quantitation of total RNA was determined by spectrometry using the value of 1 OD₂₆₀ value+40 ug RNA/ml.

Synthetic degenerate DNA primers used in the first strand cDNA synthesis (RT-PCR) and PCR amplification of the cynomolgus monkey partial cDNA.

RT-PCR primer:
EPKKAKRS-Low Reverse primer (Genset, Inc.)
5′-GTATCGATCTTTTTGCCTTCTTGGGYTC-′3

PCR Forward primer: EKYWCKW Forward primer (Genset, Inc.)

PCR Forward primer:
EKYWCKW Forward primer (Genset, Inc.)
5′-GGAATTCGARAARTAYTGGTGYAARTGG-′3

*R designates either an A or G purine base; and Y designates a either an C or T pyrimidine base.

PCR Reverse primer: EPKKAK-Low Reverse primer (Genset, Inc.)

PCR Reverse primer:
EPKKAK-Low Reverse primer (Genset, Inc.)
5′-GTATCGATCXRTTXGCRTTRTTNGGRTC-′3

*N designates either of the A, C, G or T bases; R designates either an A or G purine base; Y designates a either an C or T pyrimidine base; X designates a nucleotide analog

An oligonucleotide primer (SEQ. ID# RT-PCR primer) was used together with the SuperScript First Strand Synthesis Kit (Life Technologies, cat.#11904-018) to synthesize the first strand cDNA from 5 ug of total cynomolgus monkey RNA as per manufacturer's instructions. Briefly, 100 pmols of primer (SEQ. ID# RT-PCR primer) and 5 ug of total RNA was included in a 10 ul RT-PCR reaction, heated to 70° C. for 10 minutes, then cooled to 4° C. A 9 ul 10× RT-buffer mixture was then added to the RT-PCR reaction and incubated at 42° C. for 2 minutes, followed by the addition of 1 ul of SuperScript II enzyme to each reaction. The reverse transcription reaction allowed to proceed at 42° C. for 50 minutes. Proper control reactions were also assembled and run simultaneously. The reactions were terminated by heating to 70° C. for 15 minutes. To prevent interference of the RNA in the subsequent PCR amplification step, 1 ul of RNase H was added and the reaction incubated at 37° C. for 20 minutes before storing the single stranded cDNA material at −20° C.

1.2. Isolation, Identification and Sequencing of Simian pIgR Sequences

A 2 ul aliquot of the cynomolgus monkey cDNA reaction was used in a 50 ul PCR reaction and a partial cynomolgous double stranded cDNA amplified using 0.2 uM concentration of the Forward (SEQ. ID# PCR Forward primer) and Reverse (SEQ. ID# PCR Reverse primer) primers together with 2.5 uits of High Fidelity Platinum Taq (Life Technologies, cat.#11304-029). Amplification was carried out as per manufacturer's instructions and thermocycling conditions as follows: 1) denaturation at 94° C. for 10 minutes; 2) 30 cycles of denaturation for 1 minute at 94° C., primer annealing for 1 minute at 60° C., primer extension for 30 seconds at 72° C., and 3) a final 4° C. storage step. The correct size of the 729 bp PCR product was confirmed by agarose gel electrophoresis. The entire PCR reaction was run on a preparative agarose gel and the 730 bp partial cDNA fragment separated from contaminating primers and purified using the Qiagen QIAquick purification kit. The purified partial cDNA fragment was re-amplified and purified as described above. Due to the utilization of Taq DNA polymerase, all PCR products will contain a 3′-A overhang and will be easily ligated into an intermediate vector using the TOPO TA Cloning Kit (Invitrogen, cat.#450640), The resulting PCR product was ligated into the pCR-II vector (Invitrogen) as per manufacturer's instructions and the ligation reactions transformed into TOPO One-shot competent cells (Invitrogen). Colonies were selected and 3 ml mini-cultures grown, miniprep DNA prepared using the Qiagen Miniprep Kit (Qiagen, cat.#27106), and positive clones identified by an Eco RI restriction enzyme analysis.

Eco RI digestion identified 4 positive clones containing the PCR DNA product. Maxiprep DNA was prepared (Qiagen DNA Maxikit, cat.#12163) for two (2) clones and the DNA nucleotide sequence determined following sequencing of the DNA with both Sp6 (SEQ. ID# Sp6) and T 7 (SEQ. ID# T7) sequencing primers (SDSU Microchemical Core Facility).

1.3 Results

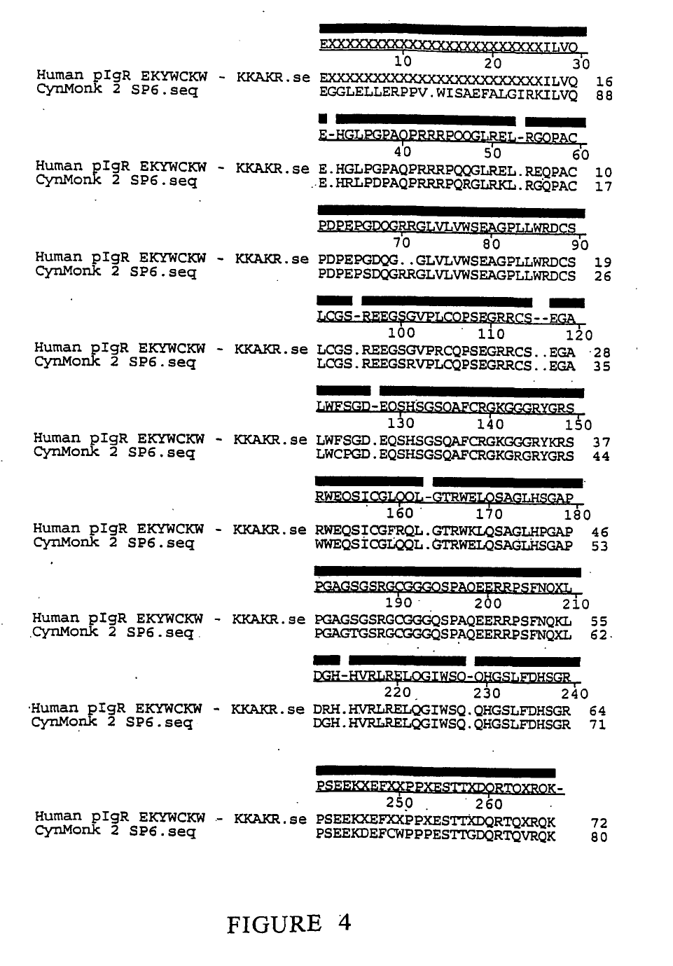

Degenerate oligonucleotides were used to clone a 730 nucleotide region of cynomolgus monkey pIgR cDNA from monkey intestinal tissue. This partial cDNA sequence encodes for most of domain 5 through the cytoplasmic domain (homologous to a region of the human pIgR molecule corresponding to amino acids Glu474 through Ser717). Detailed sequence and alignment analysis comparing the human and cynomolgus monkey pIgR cDNAs demonstrate that the sequences differ in 18 amino acids within this 242 amino acid region (Glu474 through Ser717). The nucleotide (SEQ ID NO:1) and amino acid (SEQ ID NO:2) sequences for a simian pIgR are shown in FIG. 4.

1.4. Utility

Derivatives of a polypeptide that has the amino acid sequence set forth SEQ ID NO:2, including without limitation oligopeptides, proteolytic fragments, fusion proteins, and peptidomimetics, are used as target molecules in the methods of the invention. Derivatives of a nucleic acid that has the nucleotide sequence set forth in SEQ ID NO:1 are used to produce the above polypeptides (in particular, fusion proteins) via recombinant DNA technology, and to identify and isolate nucleic acids having sequences that encode pIgR homologs from different species.

### Example 2

### Molecular Reagents

Various molecular reagents useful in the methods of the invention are derived from known and characterized pIgR targets, and from known and characterized pIgR ligands.

2.1. pIgR-Derived Target Molecules and Competitive Inhibitors

Reagents that are derived from a characterized pIgR target are used as target molecules for novel pIgR ligands, and as competitive inhibitors of the binding of pIgR targets.

2.1.1. pIgR-Derived Target Molecules

A pIgR-derived target molecule can be pIgR, fragments thereof, preferably the pIgR stalk molecule, as well as oligopeptides containing amino acid sequences from pIgR, and pIgR domains or regions that undergo apical endocytosis, reverse transcytosis, and/or basolateral exocytosis. The term “pIgR target” includes any pIgR isoform, pIgR homolog, pIgR-like protein, as well as fragments, derivatives or conjugates of any of the preceding molecules that undergo apical endocytosis, reverse transcytosis, and/or basolateral exocytosis.

2.1.2. GST-pIgR Fusion Proteins

GST-pIgR fusion proteins are one type of pIgR target molecule. The GST (glutathionine-s-transferase) polypeptide has several illustrative desirable attributes. It specifically binds glutathione, and with a sufficiently high affinity that it can be used to attach fusion proteins to solid surfaces coated with glutathione, and many such surfaces are commercially available; detectably labeled antibodies directed to GST epitopes are commercially available; and the GST amino acid sequences allow some fusion proteins to have enhanced attributes such as, e.g., enhanced solubility, biologically active conformations, and the like.

GST-pIgR fusion proteins may optionally comprise elements useful for the detection, isolation, purification and manipulation of the GST-pIgR fusion protein. Non-limiting examples of such elements include elements such as a 6×His tag, a FLAG tag, a c-myc epitope, a fluorescent polypeptide (e.g., GFP), an enzymatic polypeptide, or a biotin-binding (avidin or streptavidin) polypeptide.

GST-pIgR fusion proteins are prepared, purified and attached to solid surfaces by known techniques (see, e.g., Smith et al., Unit 16.7, “Expression and Purification of Glutathione-S-Transferase Fusion Proteins” in Short Protocols in Molecular Biology, 2nd Ed., Ausubel et al., Editors, John Wiley & Sons, pp. 16-28 to 16-31, 1992).

2.1.3 Construction of GST-Cynmonkey pIgRstalk Construct

A. Generation of a Cynomolgus Monkey pIgR Stalk Insert DNA Fragment.

A plasmid comprising cynomolgus monkey pIgR sequences (“pTA-CynMonk-pIgR,” which is a derivative of PCR pCR-II plasmid, Invitrogen, having simian pIgR sequences) is used as a template for PCR amplification of the cynomolgus monkey pIgR stalk region using the CynMpIgRstalk-5′ FOR and CynMpIgRstalk-3′REV sequencing primers. These primers allow for the use of a directional cloning strategy (BglII to EcoRI ligation) and result in the incorporation of a C-terminal His6-tag that can be used to isolate or attach the fusion protein to a solid surface.

CynMpIgRstalkGST 5′FOR
(5′-Forward PCR primer containing a Bgl II site)
5′- CG GGA AGA TCT GGA GTG AAG CAG GGC CAC TTC
TAT GG -3′

CynMpIgRstalkGST 3′REV (3′-Reverse PCR primer containing an in-frame His6-tag and Eco RI site)

CynMpIgRstalkGST 3′REV
(3′-Reverse PCR primer containing an in-frame
His6-tag and Eco RI site)
5′- CG GAA TTC CTA GTG ATG GTG ATG GTG ATG TTT GGA
GCT CCC ACC TTG TTC CTC AGA GC -3′

The 309 bp PCR fragment is purified and subject to restriction digests using EcoRI and BglII enzymes. The resulting 305 bp fragment is gel-purified. The purified EcoRI-BglII fragment is cloned into BamHI- and EcoRI-digested pGEX-2TK digested vector DNA as is described below for rabbit pIgR sequences.

B. Generation of a GST-pIgR Fusion Protein

A plasmid comprising cynomolgus rabbit pIgR sequences (“pGST-RabpIgRStalk”; for rabbit pIgR sequences, see FIGS. 2b and 2c) is digested with BamHI and EcoRI, which liberates a 312 bp fragment. The 312 bp fragment is cloned into BglII- EcoRI-treated pGEX-2TK vector, a plasmid that has a GST-encoding nucleic acid sequence that can be fused in-frame with a cloned DNA. The resulting plasmid is subjcet to DNA sequence analysis to confirm the absence of any PCR-induced mutations and to verify that the GST and pIgR sequences are linked in-frame with each other.

2.1.4. pIgR-Derived Competitive Inhibitors

In competition assays, suspected or known pIgR derivatives, isoforms and homologs are detected and/or characterized. A first, known and characterized pIgR target is used as competitive inhibitors of binding of a pIgR ligand to a second pIgR target. The pIgR-derived competitive inhibitor is detectably labeled, and the binding of a pIgR ligand thereto is detected and measured. A decrease in a signal corresponding to the binding of the detectably labeled pIgR-derived competitive inhibitor indicates an increase in the amount or extent of binding of unlabeled target molecules.

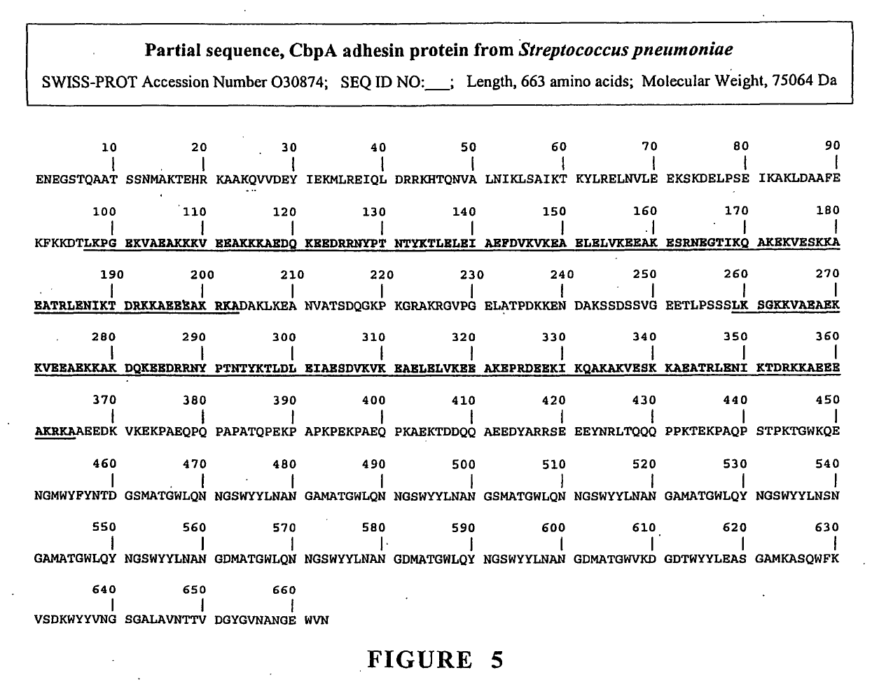

A pIgR-derived competitive inhibitor, or a collection of such molecules, is used to identify or characterize known or suspected pIgR ligands. Such methods include by way of non-limiting example the following. (1) Collections of pIgR stalk-derived oligopeptides of known sequence, some of which are competitive inhibitors of sFv-5A (an antibody derivative that binds a known epitope, i.e., QDPRLF), are used to identify the minimum amino acid sequence needed to serve as an epitope for sFv-5A. (2) Fragments derived from the bacterial protein CpbA, which is stated to bind to the secretory component (SC), are used in competition reactions to identify what portions of CpbA are required for binding (Zhang et al., Cell 102:827-837, 2000; see FIG. 5 for examples of such sequences). (3) Oligopeptides having sequences unique to sequences encoding different isoforms and homologs of a pIgR target are used to identify pIgR ligands that are specific for each isoform or homolog. (4) Oligopeptides having sequences conserved in sequences encoding different isoforms and homologs of a pIgR stalk or other pIgR molecules are “universal competitive inhibitors” that are used to identify and characterize novel pIgR, pIgR-like, pIgR stalk and pIgR stalk-like polypeptides, and other pIgR target's, including partially purified proteins, that are homologs from a variety of species of organisms, as well as isoforms and derivatives that are specific to certain cell or tissue types and/or developmental periods of an organism. (5) Oligopeptides having amino acid sequences unique to the pIgR molecule, the secretory component (SC) molecule, the pIgR stalk molecule, and other pIgR-derived pIgR target's are used to identify ligands that are specific for each of the 4 molecules.

A known, characterized pIgR stalk molecule or other pIgR target is used as a competitive inhibitor of binding of a pIgR stalk molecule or other pIgR target; this method is used to obtain novel pIgR stalk molecules and other pIgR target's. Non-limiting examples of pIgR stalk molecules and other pIgR target's that are used as competitive inhibitors include oligopeptides of from 4, 5 or 6 to about 50 amino acids having sequences derived from those of a pIgR stalk or other pIgR target, proteolytic fragments comprising amino acid sequences of a pIgR stalk or other pIgR target, and fusion proteins comprising amino acid sequences of such oligopeptides and proteolytic fragments.

2.2. Competitive Inhibitors and Target Molecules Derived from pIgR Ligands

Reagents that are derived from known or previously identified pIgR ligands are used as competitive inhibitors of the binding of novel pIgR ligands to pIgR stalk molecules and other pIgR target's, and as target molecules for novel pIgR stalk molecules and other pIgR target's.

2.2.1. Competitive Inhibitors Derived from pIgR Ligands

In some competition assays, a molecule that is or is derived from a known ligand directed to a pIgR stalk molecule or other pIgR target is used as a competitive inhibitor of the binding of other ligands. Competition assays using known competitive inhibitors of the binding of pIgR ligands are used to identify and characterize novel pIgR ligands.

2.2.2. Antibodies and Antibody Derivatives

Antibodies and antibody derivatives are one type of known pIgR ligand that can be detectably labeled and used in competition and other assays. Depending on the intended result, different types of antibody compositions are used. Compositions of polyclonal antibodies include antibodies that recognize a variety of epitopes on pIgR and may thus be useful for assays and screens where the site of interaction between pIgR and the ligand is not preselected. If it is desired to target a specific site on pIgR, monospecific or monoclonal antibodies, including derivatives thereof such as single-chain antibody fragments (sFv's), are preferred.

A non-limiting example of a known pIgR ligand is the single chain antibody sFv-5A and derivatives thereof such as sFv-5AF. As is described in more detail elsewhere herein, sFv-5A binds to an epitope having a defined amino acid sequence. When sFv-5A is used as a competitive inhibitor of pIgR ligands, the ligands whose binding will be inhibited includes those that interact with the surface of the sFv-5A epitope (competitive binding per se) and those that interact with a surface that is not the sFv epitope but is in close enough proximity thereto that bound sFv-5A precludes access to that surface (steric hinderance).

2.3. Detectable Labels and Detection Methods

The molecule or moeity that binds to pIgR is detectably labelled by any of several agents that are detectable, including, without limitation, polypeptides such as antibodies and antibody derivatives, horse radish peroxidase, alkaline phosphatase, avidin and streptavidin; radiodactive compounds containing 125I, 131I, 35S, 14C, 3H, etc.; fluorescent molecules such as fluoroscein, rhodamine and the like, and compounds that are detected by various physical techniques (i.e., spin labels).

An amino acid sequence of a pIgR ligand that is incorporated into a fusion protein may for detection purposes be joined with a fluorescent polypeptide (e.g., green fluorescent proteins, red protein), a light-emitting enzyme such as Luciferase, an enzyme such as beta-galactosidase that has a colorimetric substrate or product, a metal-binding amino acid sequence such as 6×His, an epitope that is detectable using a monoclonal antibody directed thereto (e.g., GST, c-myc, FLAG tag, and the like), an enzyme that can non-catalytically bind its substrate (GST), or an amino acid sequence that binds biotin (avidin, streptavidin).

### Example 3

### Screening Assays

The following are exemplary of screening assays of the invention.

3.1. Scintillation Proximity Assay (SPA)

3.1.1. In General

In SPA's, the detectable label on the ligand is a radioisotope that emits a particle that travels only a small distance in an aqueous solution; 125I, 33P, 35S, and 3H are suitable radioisotopes. The microsphere contains a scintillant that, when activated by particles emitted from a ligand in close proximity (i.e., bound to the surface of the spheres) emits light. The light so emitted is detected and measured by a photomultiplier. Labelled molecules that do not bind to the microspheres remain at a distance that is greater than that needed to activate the scintillant to emission of light. The method does not require the removal of unreacted chemical substrates reactants used to produce the libraries of compounds prior to screening.

3.1.2. Identification of pIgR Ligands via SPA

Fusion proteins comprising pIgR target sequences are attached to glutathione-coated microspheres by virtue of GST sequences contained therein, or to Nickel-plated microspheres by virtue of poly-His sequences therein. Other detection formats include without limitation microspheres coated with antibodies or antibody derivatives that react with tags or epitopes, such as V5, c-myc, FLAGG tag and the like that are cemically linked or otherwise associated with pIgR target molecules.

A mixture, pool or library of potential pIgR ligands is contacted with the pIgR-coated microspheres. A signal is generated from the microspheres when a radiolabeled ligand comes into close proximity to the microspheres, as occurs when labeled pIgR ligands bind the pIgR target fusion protein attached to the microspheres. As is preferable, separation of unreacted reagents is not required prior to screening.

3.2. Competition Assays

Competition assays are used to screen for and characterize pIgR ligands. Detectably labeled reagents prepared from characterized antibodies, sFv molecules or other known pIgR ligands are used in competition assays to identify novel and characterize known or suspected pIgR ligands. When present in a sample, novel and/or uncharacterized pIgR ligands compete with the detectably labeled known pIgR ligand for binding to limited number of pIgR target molecules. Competitive inhibition of the binding of a detectably labeled known pIgR ligand will result in a decrease in the signal relative to that generated when the known ligand is bound in the absence of other pIgR ligands. Changes in the signal from the detectable label are measured qualitatively to determine the presence of a pIgR ligand, or quantitatively to characterize binding attributes of known or suspected pIgR ligands.

Competition screens and assays can be designed to be used to select or enrich for pIgR ligands that bind to a particular portion of pIgR. For example, in an assay wherein immobilized pIgR is the target molecule, the inclusion of soluble secretory component (sSC) will result in a greater likelihood that ligands that bind to the immobilized pIgR are specific for the stalk. Ligands that would otherwise bind to the secretory component of the immobilized pIgR bind to the sSC and thus remain in solution.

In similar fashion, competition screens and assays can be designed to preferentially remove pIgR ligands that are cross-reactive between different species of animals. For example, the inclusion of soluble monkey pIgR target molecules in a screen for pIgR ligands that bind to immobilized human pIgR molecules would be expected to enrich for pIgR ligands that bind well to human pIgR but poorly or not at all to monkey pIgR. For screens and assays used for the pharmacological development of pIgR ligands, monoclonal antibodies and sFvs that specifically react with murine, rat, rabbit, simian or human pIgR are used.

Competition screens and assays may be designed that are specific for a particular region of or amino acid sequence within pIgR by using reagents that bind to a known region of pIgR. For example, as described herein, a single chain antibody (sFv) known as “sFv-5A” binds to a specific eptitope in pIgR. It is thus expected that pIgR ligands that competitively inhibit the binding of sFv-5A to pIgR bind to the same region of pIgR as does sFv-5A, or to a site that is in a position to enable steric hinderance of the binding of sFv-5A.

3.3. Solid State Assays

A pIgR ligand, such as a polypeptide (such as an sFv or Mab) that binds pIgR is immobilized on the wells of a multiwell (e.g., 48, 96 or more wells) plate. The polypeptide is non-specifically bound to the plates by, e.g., overnight incubation, or are specifically bound. As one example of specific binding, a pIgR polypeptide ligand is biotinylated in such a fashion that the biotinylation modification does not interfere with the ligand's interactions with pIgR. The biotinylated pIgR ligand is incubated with streptavidin coated plates. After the plates are washed, biotinylated polypeptides remain bound to the streptavidin-coated plate and are thus retained on the plates. The bound biotinylated polypeptides recognize and bind target pIgR molecules, e.g., GST-pIgR fusion proteins.

3.4. Fluorescence Polarization

A small molecule that binds to the target protein (pIgR) is labelled with a fluorescent probe. The rotational characteristics of the small fluorescent molecule are different than the unlabelled target protein. When the small fluorescent molecule binds to the target molecule its rotational characteristics take on those of the large target molecule. The small fluorescent molecule tumbles rapidly; whereas the movement of the larger target molecule is much slower. Fluorescence polarization measures the rotation of the fluorescent molecule and compares its rotation when it is free and when it is bound to the target molecule. Fluorescence polarization values range from 0 to 0.5 and are usually reported in mP (millip) units. The technique is very sensitive and is independent of fluorescence intensity, and colored solutions and cloudy solutions can be used without clarification. A minimal amount of purification of a library of molecules to be screened from the components of the reactions used to create the library is thus required before the assay is performed.

A small molecule that binds to pIgR is labelled with any of a number of fluorescent molecules, such as fluorescein, rhodamine, Texas Red, etc. Depending on the available chemical groups on the small molecule, different methods of linking the fluorescent tag to the small molecule are anticipated. Activated esters (N-hydroxysuccinimide ester), thiol groups, maleimides, and other functional groups on the parent fluorescent compounds are used to modify amines, thiols, and carboxyl groups on the small molecule that interacts with pIgR.

By incubating the small fluorescent labelled peptide with a pIgR (or GST-pIgR) target molecule, an increase in fluorescence polarization will be observed. The increase in polarization will be dependent on the concentration of the small fluorescent labelled peptide. A concentration of the peptide is chosen so that a clear and distinguishable signal is obtained. The concentration should not be so high as to make it inordinately difficult for another molecule to compete with the small fluorescent labelled peptide for the binding site on pIgR.

Competition of candidate ligands and the small fluorescent labelled peptide is used to determine the ability of the candidate ligands to bind to the same site as the small fluorescent labelled peptide. An assay using the same concentration of small fluorescent labelled peptide in all reactions with a given concentration of the candidate ligands is used to determine which, if any, of the candidate ligands can displace the small fluorescent labelled peptide from pIgR. The concentration of the candidate ligands is varied so that ‘hits’ with higher or lower affinity for pIgR are detected. Concentrations of the candidate ligand can range from picomolar to nanomolar concentrations to yield high affinity candidate ligands. If the concentrations of the candidate ligands are in the micromolar to millimolar range, then candidate ligands with lower affinity will be detected.

3.5 Detecting Signals that Vary as Intramolecular Distances Change

The interaction of either a sFv or a Mab with a particular site on the pIgR stalk involves a target and a ligand, both of which are polypeptides, and represents a non-limiting example of target:ligand interaction. Signals that vary with intermolecular distances between two or more labeled moieties are used, such as those that are detected via resonance energy transfer. If two fluorescent molecules that are suitably matched in their emission and excitation properties, and they are within a close distance, typically within 10 to 100 angstroms, and preferably within 10 to 50 angstroms, the two molecules, donor and acceptor, their interaction is detected by the quenching of the donor fluorescence and the appearance of acceptor fluorescence. The donor and acceptor molecules are matched so that the absorption spectrum of the acceptor overlaps the emission spectrum of the fluorescence of the donor. Furthermore, the dipoles of the transitions must be nearly parallel.

By labeling the Mab's or the sFv's that react with specific portions of the surface of pIgR or GST-pIgR with donor and acceptor fluorescent compounds, resonance energy changes are monitored in the presence of compounds that disrupt the transfer. Compounds that disrupt the resonance energy transfer in a competitive manner are assumed to react at or near the site of binding of the two molecules, thereby preventing them from approaching near enough to allow resonance energy transfer.

A standard set of conditions of the donor labelled pIgR and acceptor labelled sFv, for example, is monitored. By adding various concentrations of chemical libraries, natural products, peptides, etc., resonance energy measurements reveal which of these molecules disrupted resonance energy transfer. Those that disrupt and reduce resonance energy transfer are further analyzed quantitatively for the range of concentrations under which they disrupt resonance energy transfer. By this analysis, the compounds are ranked for their ability to bind to the pIgR binding site. A compound may react with pIgR or with the sFv, either interaction would disrupt resonance energy transfer.

Additional characterization using direct binding assays, such as calorimetry, and such as calorimetry and stopped flow methods using absorbance, fluorsescence, light scattering, turbidity, fluorescence anisotropy, etc. or equilbrium dialysis, demonstrates that the compound reacts with pIgR. Additional rapid separation methods that separate complexes from unbound ligand provide the quantitative basis for analysis of binding constants (Kd) (Flass, T., Biospecific binding and kinetics—A fundamental advance in measurement technique, Biomedical Products 20, 122-123, 1995). One such analysis is performed on the Sapidyne KinExA 3000 instrument. Derivatives of the candidate compound candidate are made to determine which protein, pIgR or sFv, was the target. A fluorescent derivative could be used in fluorescence polarization studies. A biotinylated derivative could be used in ELISA assays or bead capture assays (streptavidin-beads).

### Example 4

### Isolation of Monoclonal Antibodies Directed to a Preselected Portion of pIgR

4.1. ELISA for Monoclonal Antibodies

An assay is prepared by applying purified pIgR stalk molecules or GST-pIgR stalk molecules, or any other pIgR target, to multiwell (48-well, 96-well and other size plates and allowing the protein to adhere to the wells of the plates during overnight incubation. The plates are washed to remove unbound proteins. Samples of the serum from the immunized mice are incubated with the pIgR or GST-pIgR coated plates. After 1 to 2 hours of incubation (gentle shaking at room temperature), the plate is washed free of unreacted immune serum proteins. Mouse antibodies that react with an immobilized GST-pIgR protein are detected by adding to each well a sample of a goat antibody that has been raised against and is directed to mouse immunoglobulin, i.e., all subclasses of murine immunoglobulins. The goat antibody is conjugated to an enzyme that is used for detection; non-limiting examples include horse radish peroxidase and alkaline phosphatase. After unreacted horse radish peroxidase or alkaline phosphatase conjugated goat anti-mouse immunoglobulin has been washed from the wells, the substrate of horse radish peroxidase or alkaline phosphatase is added. When the color is sufficiently developed, the reaction is stopped and quantitated using a spectrophotometer. In the positive wells, antibodies against the GST-pIgR protein will be present. Some of these antibodies are directed to the GST portion of the protein if GST-pIgR is used. By assaying against other GST fusion proteins, it is determined if the antibodies are against GST or pIgR. This assay is also used to identify antibody producing cells and clones in 96-well plates that are part of the process of isolating clones of hybridomas that produce the desired monoclonal antibody.

Beads that bind GST moieties on GST-fusion proteins are also used for assays. GST-pIgR bound to beads is reacted with sera that contain antibodies directed against pIgR. The antibodies that react with and bind to pIgR can then be detected by an anti-antibody conjugated to horse radish peroxidase or alkaline phosphatase. If the antibodies that react with pIgR are derived from mice, then the antibodies that detected the presence of the mouse antibody is obtained from another animal species, such as goat or sheep. Those skilled in the art will know how to adjust the source and specificity of the detecting antibody conjugates (i.e. horse radish peroxidase or alkaline phosphatase conjugated to anti-FLAG tag antibody) to obtain the desired results.

4.2. Preparation of Monoclonal Antibodies (Mabs)

Monoclonal antibodies are created by immunizing mice with portions of pIgR, generally prepared as oligopeptides having defined amino acid sequences. For example, a nucleic acid encoding an amino acid sequence found in a conserved region of pIgR, such as those described in Table 2, or a amino acid sequence that varies between homolgs, such as, e.g., R1, R2a, R2b, R3a, R3b, R3c (etc.) is used to create a pIgR-target-GST fusion protein that is expressed in a host cell such as E. coli. The GST portion of the fusion protein is used to isolate the fusion protein, and the purified GST-pIgR protein is mixed with adjuvant and injected into mice to produce an immune response. The extent of the immune response is measured over time by removing blood from the immunized mice at regular intervals and measuring the level of antibodies directed to the GST-pIgR fusion protein using an immunoassay, e.g., an ELISA.

Once the immunized mouse has been shown to be producing antibodies directed to the GST-pIgR fusion protein, the spleen of the mouse is harvested, and cells therefrom are prepared for fusion with immortalized fusion partners, such as the NS/1 cell line, according to Kohler and Milstein, in order to create Mab-producing hybridoma cell lines. Independently isolated clones and subclones are grown to an appropriate density, the cell supernatant is assayed using an ELISA to determine if antibodies that react with the GST-pIgR fusion proteins are produced by each clone or subclone. Positive wells are assayed using limiting dilution, and clonal and subclonal cell lines are eventually obtained that produce Mabs against either the GST-pIgR fusion protein.

By assaying and comparing results from assays using commercially available monoclonal antibodies directed to GST, and GST fusion proteins that do not contain pIgR, as well as polyclonal antibodies directed to pIgR, it is possible to identify isolated Mabs that either are pIgR specific or are specific to an epitope not present in either pIgR or GST but which occurs at the junction thereof. The Mabs can additionally be tested for specificity using MDCK cells and MDCK cells that have been transfected with different species of pIgR (human, rat, mouse, pig, rabbit, monkey, etc.).

The goal is to assemble a large panel of monoclonal antibodies and sFvs that cumulatively bind to every part of the surface of pIgR domain 6, which includes the pIgR stalk. Each of the sFvs and the Mabs are epitope mapped using the nested set of overlapping 15 ′mer peptides. Linear epitopes and conformational epitopes are identified on the strength of their binding and the location of the peptides in the nested set.

### Example 5

### Single Chain Antibodies Directed to Transcytotic Molecules and pIgR Target Molecules

5.1. Single-Chain Antibody Fragments (sFv's) Directed to pIgR

A useful pIgR ligand that may be used as a reagent is an antibody directed to pIgR, or an active fragment or derivative of such an antibody. Any sFv that binds to pIgR may be used in the methods to identify small molecules that bind to pIgR.

As one example of a useful sFv, sFv-5A is human sFv that recognizes an epitope on human and rat polymeric immunoglobulin receptor (pIgR). The sFv-5A compound is described in U.S. patent application Ser. No. 09/818,247 (attorney docket no. 18062E-000900 entitled “Ligands Directed to the Non-Secretory Component, Non-Stalk Region of pIgR and Methods of Use Thereof” by Mostov, Keith E., and Chapin, Steven J.), filed Mar. 27, 2000, describes the B region of pIgR and ligands directed to the B region of pIgR; and U.S. patent application Ser. No. 60/192,198 (attorney docket no. 18062E-003000US entitled “Anti-pIgR Antibodies With Improved Transcytosis by Mostov, Keith E., Chapin, Steven J., and Richman-Eisenstat, Janice), filed Mar. 27, 2000

Another type of sFv of note is one that binds the secretory component (SC) and is thus useful to distinguish between ligands that bind that molecule and those that bind the pIgR stalk molecule. One example of an SC-directed sFv′ is taught in U.S. Pat. No. 6,072.

5.2. Preparation of Phage Display Libraries

Libraries of polypeptides are displayed on gene III, gene VIII, or gene VI on filamentous fd phage. The polypeptides are linear and may contain cysteine residues that will form disulfide bonds to restrict the number of conformations that the peptide may assume. An even number of cysteines is used for this purpose, 2 or 4 being preferred. The cysteines are closely spaced to form a loop of 3 to about 20 residues held between the cysteines. The cysteines are closely spaced to further immobilize the peptide conformation, such as in A-Cys-Y-Cys-X-Cys-Z-Cys-B, where A and B are independently selected amino acid sequences that are from 1 to 10 residues long that link the polypeptide to the filamentous phage and provide flexibility; Y is 0 to 5; Z is 0 to 5; and X is 3 to about 20. The X region is an immobilized loop. At each position except for the cysteine positions, any amino acid is placed to produce a completely random peptide sequence containing all possible amino acid sequence combinations. Amino acids in the Y and Z regions are generally smaller in size compared to tryptophan to allow cystine bonds to form. Also within the present invention are linerar peptides 4 to about 20 amino acids in length that are displayed on gene III, gene VIII, or gene VI on filamentous fd phage.

5.3. Phage Display Selection for pIgR Ligands

Microtiter plates that contain immobilized pIgR-derived target molecules are prepared. Preferred plates are those having wells large enough to accept 0.2 to 3 mls of a liquid.

The GST portion of the GST-pIgR fusion protein described herein is used to bind the fusion protein to solid surfaces that are coated with, e.g., commercially available monoclonal antibodies that are directed to a GST-specific antigen, or GST's natural substrate, glutathione. Alternatively, a pIgR target molecule that contains a N- or C-terminal cysteine at the end of a spacer may be attached to a solid surface via a chemical bond. Plates that contain maleimido groups to which the cysteine thiol can react are used for this purpose. Alternatively, the cysteine thiol is reacted with a maleimido-biotin compound in order to covalently link a biotin moiety to a pIgR target; a plate coated with streptavidin or avidin is then used to bind biotinylated pIgR target molecules. A plate that contains a thiol group to which a cysteine thiol can react is also used. The cysteine thiol on the plate is reacted with thiol containing compounds to covalently link the compount

A phage preparation containing a pool or library of random peptides is contacted with plates coated with immobilized pIgR. The plates are gently agitated on a rotary table at room temperature for a preselected period of time. After the plates are rapidly washed several (3 to 5) times with buffer, any remaining (pIgR-bound) phage are removed using a solution of urea or other appropriate reagent (e.g., an excess of free gluatathione in instances where a GST-pIgR fusion protein is bound to glutathione-coated beads). The phage are collected, concentrated and freed of urea and any other undesirable components, and grown on E. coli host cells to increase their number. This selection process is carried out additional times to further increase the specificity of ligands that are selected by the screening assays. Three to ten rounds of selection reduce the number of phage that (i) react with pIgR and (ii) contain a unique sequence of amino acids. After the desired number of selection rounds has been performed, several (10 to 25) phage plaques are selected randomly from a lawn of E. coli that has been overlaid with the phage and the sequence of the amino acid residues in the peptide determined by DNA sequencing. The design of the members of the phage library is such that oligonucleotide primers can be used for the purpose of sequencing through the amino acids of the random peptide.

It is possible that more than one amino acid sequence will be isolated; however, there are similarities among the sequences that suggest certain residues in the sequence are found more frequently and are, therefore, likely to be residues that interact directly with the pIgR moiety.

5.4. ELISA Assay for sFv Supernatants

As described above, an assay is constructed by applying the purified pIgR or GST-pIgR to multiwell, preferably 48- or 96-well plates, and allowing the protein to adhere to the wells during overnight incubation. The excess proteins are removed by washing, and supernatents of E. coli that contain filamentous phage that express sFv as a secreted protein and which are part of a sFv library, for example, are incubated with the pIgR or GST-pIgR coated plates. Alternatively, soluble extracts of E. coli that express sFv intracellularly are contacted with the pIgR- or GST-pIgR-coated plates.

After incubation and washing, sFv's that remain bound to the immobilized GST-pIgR protein are detected by adding to each well an antibody that binds humans sFv's in general, and detecting the signal thereby amplified, or by detecting a signal from a detectable label that has been attached to an invariable portion of the sFv gene and thus is present in every member of a collection. The detection is performed essentially as in the preceding section on Elisa assays on monoclonal antibodies. Those skilled in the art will know how to adjust the source of the detecting antibody conjugates (i.e. horse radish peroxidase or alkaline phosphatase) to obtain the desired results. Luminescence and chemiluminescence is also used. Substrates such as 1,2_dioxetane contain phosphate groups that are cleaved and produce chemiluminescence.

Beads that bind GST moieties on GST-fusion proteins are also used for assays. GST-pIgR bound to beads is reacted with solutions containing sFvs directed against pIgR. The sFvs that react with and bind to pIgR are then detected by an anti-sFv or anti-tag antibody conjugated to horse radish peroxidase or alkaline phosphatase.

### Example 6

### Phage Displaying Polypeptides for Penetrating Epithelial Cell Layers

The present example provides methods and compositions for isolating phage that undergo reverse (apical to basolateral) transcytosis in epithelial cells or paracellular transport, i.e, transport through gap junctions found between epithelial cells. More generally, the disclosure provides methods and compositions for isolating phage that undergo any type of active or passive transport across an epithelial cell or through an epithelial barrier.

Techniques for selecting of phage that are capable of cellular internalization have been described by Becerril et al. (Biochem. Biophys. Res. Commun. 255, 386-393, 1999) and Ivanenkov et al., (Biochimica et Biophysica Acta 1448 (1999) 450-462; Id., 463-472). Ivanenkov and Menon describe techniques used to isolate phage that transcytose from a basolateral compartment to an apical compartment that were used to identify peptide ligands that bind the an integrin protein (Biochem. and Biophys. Res. Commun. 276, 251-257, 2000).

In the present example, MDCK cells are grown on porous transwell membranes. The porous membrane separates the two cellular compartments, the basolateral compartment and the apical compartment. The basolateral compartment is the membrane side on which the cells rest. The apical side of the MDCK cell layer does not contact the porous membrane. The MDCK cells, or other appropriate cells known to those skilled in the art, form a continuous and contiguous layer that is substantially impermeable to large molecules unless a specific transport system exists for those substances.

MDCK cells may be transfected with various homologs, isoforms and derivatives (e.g., fusion proteins) of pIgR so that the pIgR is expressed and displayed on the surface of the cells. Expression vectors comprising any homologs of pIgR may be transfected into MDCK cells. Preferred pIgR homolgs include rat, mouse, monkey, rabbit, simian and human pIgR. Chinese Hamster Ovary (CHO) cells transfected with expression vectors for mouse pIgR are described by Asano et al. (J. Immunol. Methods 214, 131-139, 1998); and transgenic mice that overexpress murine pIgR are described by de Groot et al. (Transgenic Res. 8, 125-135,1999).

Other epithelial cells may be grown on transwell membranes and used to identify peptides and proteins that enhance the transcytosis or paracelullar transport of phage. Such cells may or may not express pIgR. In cells where pIgR is not present, another type of transcytocic or paracellular transporting element or mechanism casuses, promotes, enhances or mediates trancytosis or paracellular transport of phage that display certain polypeptides.

Phage are placed in the apical compartment and isolated in the basolateral compartment. Phage that penetrate the cell layer are found in the basolateral compartment as opposed to the apical compartment into which they were initially introduced. Although phage may be guided around the cell and through the junctions of adjacent cells (paracellular transport), phage will also be internalized and further guided across the cell by transcytotic processes, including processes involving pIgR targets.

The media of the basolateral compartment is diluted and plated on a lawn of E. coli host cells. Each individual phage produces a plaque (a circular zone of lysis) that results from the replication and growth of the phage; each plaque thus contains many copies of a specific phage. The number of phage isolated using control phage that lack displayed peptides and proteins is compared to the phage displaying candidate liquids. The difference in the number of control and experimental phage reflects the presence of transcytotic phage in the latter.

By amplifying the number of phage in successive rounds, preferably 3 to 6 rounds, or cycles of selection, an enriched population of phage displayed peptides and proteins is produced. The amino acid sequences of polypeptides that confer the properties of being able to penetrate the epithelial cell layer, to undergo paracellular transport, and/or apical to basolateral transcytosis are identified as follows. DNA is isolated from a phage population that is grown in E. coli cells in suspension culture, and the portion of the phage DNA that encodes the polypeptide displayed thereby is determined. The nucleotide sequence is translated in silico to yield an amino acid sequence that is or comprises a sequence that confers upon the phage the ability to undergo active or passive transport across an epithelial cell or through an epithelial barrier. A polypeptide comprising the amino acid sequence is prepared by, e.g., in vitro chemical synthesis or recombinant DNA technologies.

Individual phage are mutagenized in order to generate focused libraries of amino acid sequences that confer transcytotic or paracellular transporting properties, or of polypeptides that undergo any type of active or passive transport through an epithelial cell or barrier. Similarly, the amino acid sequences identified in this manner can be used as candidate compounds that can be chemically modified and otherwise derivatized in order to yield polypeptides and peptidomimetics having such desirable atttributes.

Polypeptides that confer transcytotic or paracellular transporting properties, or polypeptides that undergo any type of active or passive transport through an epithelial cell or barrier, are used to identify molecules within the epithelial layer that mediate such processes. Polypeptides from that contain amino acid sequences from a ligand displayed by a phage that confer transcytotic or paracellular transporting properties to the phage are detectably labeled by, e.g, iodination using ICl, Chloramine T, Iodobeads, Bolton-Hunter reagent or other appropriate reagents. Fluorescent labels may also be covalently attached to polypeptides in order to render them detectably labeled.

Identification of Cellular Transport Molecules

The detectably labeled polypeptides are chemically cross-linked to preparations of cells including without limitations lysates, cell membrane preparations, mitochondrial preparations, and the like. Cell membrane preparations are preferred for the identification of cellular surface molecules. Limited chemical or enzymatic digestion of the cross-linked cellular molecules are prepared, and molecules therefrom are resolved by electrophoresis. Cellular molecules, or fragments thereof, to which the ligand is attached are thus detectably labeled, and are further characterized. For example, for a cell-displayed polypeptide, amino acid sequences present in the labeled cross-linked material are determined. Although a complete polypeptide sequence is not necessarily thereby obtained, such amino acid sequences are used to design nucleic acids (probes, PCR primers) that are used to identify and characterize nucleic acids that encode the entire polypeptide.

Polypeptides that confer the property of being able to penetrate epithelial cell barriers and/or transcytotic or paracellular transporting properties to phage are prepared using the amino acid sequence of the phage-displayed protein. The polypeptides are incorporated into fusion proteins, chemically linked or conjugated or otherwise brought into association with therapeutic compounds so as to create therapeutic compounds that cross an epithelium, preferably an epithelium facing the lumen of an organ, most preferably the pulmonary lumen or the gastrointestinal lumen. Methods of preparing fusion proteins and chemical conjugates comprising pIgR ligands are described in U.S. patent application Ser. No. 60/237,929 (attorney docket No. 030854.0009 entitled “Genetic Fusions of pIgR Ligands and Biologically Active Polypeptides for the Delivery of Therapeutic and Diagnostic Proteins” by Houston, L. L., Glynn, Jacqueline M., and Sheridan, Philip L.), filed Oct. 2, 2000;and U.S. patent application Ser. Nos. 60/248,478 and 60/248,819 (attorney docket Nos. 030854.0009.PRV2 and 030854.0009.PRV3 entitled “Protein Conjugates of pIgR Ligands for the Delivery of Therapeutic and Diagnostic Proteins” by Houston, L. L., and Hawley, Stephen), filed Nov. 13, 2000 and Nov. 14, 2000 respectively, and these applications are incorporated in their entirety herein.

### Example 7

### Small Molecule pIgR Ligands

7.1. Compound Libraries

Libraries of a variety of types of molecules are prepared in order to obtain members therefrom having one or more preselected attributes that are prepared by a variety of techniques, including but not limited to parallel array synthesis (Houghton, Annu Rev Pharmacol Toxicol 2000 40:273-82, Parallel array and mixture-based synthetic combinatorial chemistry; solution-phase combinatorial chemistry (Merritt, Comb Chem High Throughput Screen 1998 1(2):57-72, Solution phase combinatorial chemistry, Coe et al., Mol Divers 1998-99;4(1):31-8, Solution-phase combinatorial chemistry, Sun, Comb Chem High Throughput Screen 1999 2(6):299-318, Recent advances in liquid-phase combinatorial chemistry); synthesis on soluble polymer (Gravert et al., Curr Opin Chem Biol 1997 1(1):107-13, Synthesis on soluble polymers: new reactions and the construction of small molecules); and the like. See, e.g., Dolle et al., J Comb Chem 1999 1(4):235-82, Comprehensive survey of cominatorial library synthesis: 1998. Freidinger R M., Nonpeptidic ligands for peptide and protein receptors, Current Opinion in Chemical Biology; and Kundu et al., Prog Drug Res 1999;53:89-156, Combinatorial chemistry: polymer supported synthesis of peptide and non-peptide libraries). Compounds may be clinically tagged for ease of identification (Chabala, Curr Opin Biotechnol 1995 6(6):633-9, Solid-phase combinatorial chemistry and novel tagging methods for identifying leads).

Libraries containing oligosaccharides have been made in solution and by solid phase synthesis (Schweizer F. Hindsgaul O., Combinatorial synthesis of carbohydrates, Current Opinion in Chemical Biology. 3:291-8, 1999). The combinatorial synthesis of carbohydrates has been described (Schweizer et al., Curr Opin Chem Biol 1999 3(3):291-8, Combinatorial synthesis of carbohydrates). The synthesis of natural-product based compound libraries has been described (Wessjohann, Curr Opin Chem Biol 2000 4(3):303-9, Synthesis of natural-product based compound libraries).

Libraries of nucleic acids are prepared by various techniques, including by way of non-limiting example the ones described herein for the isolation of aptamers. Libraries that include oligonucleotides and polyaminooligonucleotides (Markiewicz et al., Synthetic oligonucleotide combinatorial libraries and their applications, Farmaco. 55:174-7, 2000) displayed on streptavidin magnetic beads are known and are particularly useful in some aspects of the invention. Nucleic acid libraries are known that can be coupled to parallel sampling and be deconvoluted without complex procedures such as automated mass spectrometry (Enjalbal C. Martinez J. Aubagnac J L, Mass spectrometry in combinatorial chemistry, Mass Spectrometry Reviews. 19:139-61, 2000) and parallel tagging. (Perrin D M., Nucleic acids for recognition and catalysis: landmarks, limitations, and looking to the future, Combinatorial Chemistry & High Throughput Screening 3:243-69).

Peptidomimetics are identified using combinatorial chemistry and solid phase synthesis (Kim H O. Kahn M., A merger of rational drug design and combinatorial chemistry: development and application of peptide secondary structure mimetics, Combinatorial Chemistry & High Throughput Screening 3:167-83, 2000; al-Obeidi, Mol Biotechnol 1998 9(3):205-23, Peptide and peptidomimetric libraries. Molecular diversity and drug design). The synthesis may be entirely random or based in part on a known polypeptide.

Polypeptide libraries are prepared according to various techniques that are described in more detail throughout the specification. In brief, phage display techniques can be used to produce polypeptide ligands (Gram H., Phage display in proteolysis and signal transduction, Combinatorial Chemistry & High Throughput Screening. 2:19-28, 1999) that may be used as the basis for synthesis of peptidomimetics. Polypeptides, constrained peptides, proteins, protein domains, antibodies, single chain antibody fragments, antibody fragments, and antibody combining regions are displayed on filamentous phage for selection.

Large libraries of individual variants of human single chain Fv antibodies have been produced. See, e.g., Siegel R W. Allen B. Pavlik P. Marks J D. Bradbury A., Mass spectral analysis of a protein complex using single-chain antibodies selected on a peptide target: applications to functional genomics, Journal of Molecular Biology 302:285-93, 2000; Poul M A. Becerril B. Nielsen U B. Morisson P. Marks J D., Selection of tumor-specific internalizing human antibodies from phage libraries. Source Journal of Molecular Biology. 301:1149-61, 2000; Amersdorfer P. Marks J D., Phage libraries for generation of anti-botulinum scFv antibodies, Methods in Molecular Biology. 145:219-40, 2001; Hughes-Jones N C. Bye J M. Gorick B D. Marks J D. Ouwehand W H., Synthesis of Rh Fv phage-antibodies using VH and VL germline genes, British Journal of Haematology. 105:811-6, 1999; McCall A M. Amoroso A R. Sautes C. Marks J D. Weiner L M., Characterization of anti-mouse Fc gamma RII single-chain Fv fragments derived from human phage display libraries, Immunotechnology. 4:71-87, 1998; Sheets M D. Amersdorfer P. Finnern R. Sargent P. Lindquist E. Schier R. Hemingsen G. Wong C. Gerhart J C. Marks J D. Lindquist E., Efficient construction of a large nonimmune phage antibody library: the production of high-affinity human single-chain antibodies to protein antigens [published erratum appears in Proc Natl Acad Sci USA 1999 96:795], Proceedings of the National Academy of Sciences of the United States of America 95:6157-62, 1998).

Focused or smart chemical and pharmacophore libraries are designed with the help of sophisticated strategies involving computational chemistry (e.g., Kundu B. Khare S K. Rastogi S K., Combinatorial chemistry: polymer supported synthesis of peptide and non-peptide libraries, Progress in Drug Research 53:89-156, 1999) and the use of structure-based ligands using database searching and docking, de novo drug design and estimation of ligand binding affinities (Joseph-McCarthy D., Computational approaches to structure-based ligand design, Pharmacology & Therapeutics 84:179-91, 1999; Kirkpatrick D L. Watson S. Ulhaq S., Structure-based drug design: combinatorial chemistry and molecular modeling, Combinatorial Chemistry & High Throughput Screening. 2:211-21, 1999; Eliseev A V. Lehn J M., Dynamic combinatorial chemistry: evolutionary formation and screening of molecular libraries, Current Topics in Microbiology & Immunology 243:159-72, 1999; Bolger et al., Methods Enz. 203:21-45, 1991; Martin, Methods Enz. 203:587-613, 1991; Neidle et al., Methods Enz. 203:433458, 1991; U.S. Pat. No. 6,178,384).

7.2. Screening Assays

Preferred collections of potential candidate compounds are prepared in a homogenous reaction mixture, i.e., one in which separation of unreacted reagents from members of the library is not required prior to screening.

Detectable labels such as fluorescent tags are added to molecules using reactive agents that chemically modify amino, carboxyl, and thiol groups on the small target molecules. Small focused libraries are made around a pharmacophore that has already been identified to react with pIgR. Once a pharmacophore has been identified using a fluorescent tagged molecule, the fluorescent tag is removed (i.e., the pharmacophore is resynthesized without the tag) and the candidate pIgR ligand is characterized using, e.g., competitive assays.

Combinatorial libraries can are also detectably labeled with biotin. Various forms of biotin are available to react with amino, carboxyl, and thiol groups on small molecules. These biotinylated compounds are used in ELISA formats to identify small molecule pharmacophores that react with pIgR.

### Example 8

### Assays for the in vivo Delivery of pIgR-Targeted Compounds

A variety of assays are used to determine the extent of delivery of a pIgR targeted fusion protein from the lumen of an organ to the body of an animal. Non-limiting examples of such organs are the gastrointestinal tract and the lung.

For example, in order to determine the delivery of pIgR targeted fusion proteins from the gastrointestinal tract is determined according to the following procedures. A cannula is implanted into the jugular vein of a rat for the purpose of collecting blood samples at various times. Another cannula is implanted into a region of the intestine, jejunum, ileum, or colon, for the purpose of administering the therapeutic entity to the intestine. A 350-375 gram Spraque-Dawley rat is suitable for this purpose although other strains of rats may be used. The cannulae are guided under the skin so that they exit the skin directly between the shoulders of the rat. This position prevents the rat from damaging the cannula. A single rat per cage is required. The fusion protein is administered to the rat 2 to 7 days after the cannulae are implanted. During this time, the rat is observed for its general health and to determine the patency of the cannulae.

The therapeutic entity is given to the rat through the intestinal cannula. Before administration, a sample of blood (approximately 200 microliters) is withdrawn through the jugular vein cannula. Samples of blood are collected over a 8 to 48 hour period. The jugular cannula is kept patent by using saline with a small amount of heparin to prevent clotting. The blood is collected into a 1.5 ml Eppendorf tube that contains 5 microliters of heparin to prevent clotting. The blood is kept on ice for up to 1 hour, but no longer, before it is centrifuged in a table top Eppendorf centrifuge for 30 to 60 seconds. The supernatant is collected (plasma) and stored in a suitable manner, usually by freezing at −80° C.

The presence and amount of the fusion protein is measured using any appropriate assay. For example, the fusion protein is radioiodinated using 125I using any of the usual methods of radioiodination that are known to those skilled in the art. These methods include using chloramine-T, immobilized chloramine-T, iodine monochloride, lactoperoxidase beads, or lodogen.

Radioiodinated fusion proteins are separated from unreacted 125I by chromatography, including size separation on Sephadex or Sepharose, or by dialysis. The weight of the blood is determined by collecting the blood into a preweighed Eppendorf or small glass tube and determining the weight of the blood by subtraction after weighing the tube containing the blood. The entire tube may be counted in a gamma counter and the number of counts per minute divided by the weight of the blood to determine the number of cpm per gram of blood (essentially equivalent to the cpm/ml of blood). A graph of the cpm/ml of blood as a function of time after administration of the radiolabelled therapeutic entity is used to illustrate the transport of the therapeutic entity from the intestine into blood.

Plasma may be obtained by collecting the blood into 5 microliters of heparin (about 5 to 50 units/ml) and centrifuging the tube. The fusion protein plasma may be examined to determine if it has the same molecular weight by SDS-PAGE. A sample of the plasma may be compared on SDS-PAGE with a sample of the radiolabelled therapeutic entity that was administered through the cannula. If the patterns of radioactivity (autoradiography) are the same, then it is concluded that the fusion protein that is present in blood is not degraded. The blood sample is reacted to immunoprecipitate the therapeutic entity. The immunoprecipitated sample is compared to an immunoprecipitated sample from the stock radiolabelled fusion protein by separation and visualization on SDS-PAGE. A quantitative estimate of the amount of therapeutic entity is made by comparing the amount of cpm that was immunoprecipitated from blood samples and from stock radiolabelled fusion protein.

An immunoassay such as, for example, an enzyme linked immunosorbent assay (ELISA) is used to determine the concentration of the fusion protein. In this case, the fusion protein is not radiolabelled. An antibody that recognizes an epitope present in the fusion protein or in an optional detectable element thereof, is coated to the bottom of 96-well plates. After washing, the presence and quantity of the fusion protein is determined by reacting it with a second antibody that is conjugated to a detectable enzyme such as, e.g., horse radish peroxidase or alkaline phosphatase. After washing, a substrate for horse radish peroxidase or alkaline phosphatase is incubated in the well. Substrate is detectable or results in a detectable product. The amount of the product determined by spectrophotometry at an appropriate wavelength. A control curve (using known quantities of the fusion protein) is used to determine the concentration of the fusion protein in the plasma samples.

Similar experiments are conducted to examine a fusion protein's potential as a component as a component of a composition intended for rectal delivery, e.g., a suppository. In these experiments, the fusion protein is administered by a rectal tube. A catheter is inserted through the anus of an anesthetized rat. The urinary catheter inserted 7.5 cm through the anus will result in delivery within the colon.

Similarly, the above described procedures can be used to examine a fusion protein's potential for administration via inhalation. In these experiments, the fusion protein is administered as an aerosol or microparticulate formulation to the nasal or pulmonary cavity.

### Example 9

### In vivo Testing of Ligands Directed to Transcytotic Receptors

Rat cancer models are used to determine the efficacy of therapeutic agents comprising transcytotic properties, including agents such as pIgR-targeted fusion proteins (for an example of the application of such methods, see Beneditti et al., Cancer Res. 59:645-652, 1999). For example, a pIgR-targeted fusion protein that includes a biologically active polypeptide reacts with epidermal growth factor receptor, (EGFR) is tested for its ability to inhibit the growth of tumors implanted into a rat. If the biologically active polypeptide reacts with rat EGFR, the tumor cells that are implanted are of rat origin and grown in a wild type rat. If the biologically active polypeptide reacts with human EGFR, the tumors cells that are implanted are of human origin and are grown in an immune compromised (scid) rat. The rat is prepared for administration of the therapeutic entity by inserting a cannula into a region of the intestine, such as the jejunum, ileum, or colon. After the surgery required to insert the cannula, the rat is optionally allowed to rest for 2 to 7 days to recover. During this time, the rat is observed for its general health and the patency of the cannula. During this time, or shortly before the surgery, tumor cells are injected subcutaneously into the flank of the rat. Depending on the specific tumor cell line used and its ability to form tumors, 10,000 to 5,000,000 cells are injected subcutaneously. The cells are first grown in tissue culture medium and then taken up as a suspension. The cells are injected into the animal subcutaneously.

The tumor cells are allowed to grow for 5 to 14 days before the tumor is treated with the fusion protein administered through an intestinal cannula in a formulation appropriate for the gastrointestinal tract. Alternatively, formulations for the inhalation delivery of proteins are tested via administered through the pulmonary or nasal cavity using an aerosol.

The EGFR-expressing cell line TE8, an esophageal squamous cell carcinoma, and the EGFR-deficient cell line H69 may be used to determine the efficacy of the fusion protein. (Suwa et al., International Journal of Cancer. 75:626-634, 1998). The A431 cell line, a human epidermoid carcinoma tumor cell line, may also be used to test the effects of the fusion protein. The A431 cells are grown in athymic rodents, including rats.

Measurements of the tumor size are made using calipers to measure the dimensions of the tumor in two directions. The volume of the tumor is determined by multiplying the longest dimension times the square of the shortest dimension and dividing the product by 2. By plotting the tumor volume as a function of time (using the average or mean tumor volume) for a group of rats given the fusion protein, and comparing the same plot for a group of untreated rats bearing a tumor prepared in the same manner, one skilled in the art can determine the ability of the fusion protein to inhibit or slow the growth of the tumor and preferably, to eradicate the tumor.

The EGFR-expressing cell line TE8, an esophageal squamous cell carcinoma, and the EGFR-deficient cell line H69 may also (Id.) Athymic nude rats bearing orthotopically implanted LNCaP tumors may be implanted subcutaneously and treated with the fusion protein. (Rubenstein et al., Medical Oncology 14:131-136, 1997).

Tumor cells, such as C6 cells, may also be implanted stereotactically into the right caudate nucleus of Wistar rats. A cannula into the intestine may also be put into these rats for the purpose of administering the fusion protein. Rats with well-established cerebral C6 glioma foci may be given the fusion protein through the intestinal cannula. The mean survival time of tumor bearing rats is about 15-20 days in this model. The efficacy of the fusion protein may be measured by comparing the life span of control rats (i.e, tumor bearing rats given no fusion protein) to rats given the fusion protein (Pu et al., Journal of Neurosurgery 92:132-139, 2000).

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent. applications, or other documents.

SEQUENCE LISTING

```
SEQ ID NO: Partial cDNA sequence of pIgR from
cynomolgus monkey (Macaca fascicularis).
+1
GAGAAGTATTGGTGTAAGTGGAGTAACACAGGCTGCCAGA
CCCTGCCCAGCCAAGACGAAGGCCCCAGCGAGGCCTTCGT
AAACTGTGACGAGAACAGCCGGCTTGTCTCCCTGACCCTG
AACCCAGTGACCAGGGCAGACGAGGGCTGGTACTGGTGTG
GAGTGAAGCAGGGCCACTTCTATGGAGAGACTGCAGCTGT
CTATGTGGCAGTTGAAGAGAAGAAGGTAGCAGGGTCCCGC
TATGTCAGCCCAGCGAAGGCAGACGCTGCTCCTGATGAGA
AGGTGCTAGACTCTGGTGTCCGGGAGATTGAGAACAAAGC
CATTCAGGATCCCAGGCTTTTTGCAGAGGAAAAGGTCGTGG
CAGATACGGGAGATCAAGCTGGTGGGAGCAGAGCATCTG
TGGATTCCAGCAGCTCTGAGGAACAAGGTGGGAGCTCCAA
AGCGCTGGTCTCCACTCTGGTGCCCCTGGGCCTGGTGCTG
GCACTGGGAGCCGTGGTTGTGGGGGTGGCCAGAGCCCGG
CACAGGAAGAACGTCGACCGAGTTTCAATCAGAAGCTACAG
GACAGACATTAGCATGTCAGACTTCGAGAACTCCAGGGAAT
TTGGAGCCAATGACAACATGGGAGCCTCTTCGATCACTC
AGGAGACATCCCTCGGAGGAAAAGATGAGTTTGTTGCCAC
CCCTGAGAGCACCACGGAGACCAAAGAGCCCAAGAAGGCA
AAAAGATCG
                729
SEQ ID NO:2 Amino acid sequence of pIgR from
cynomolgus monkey (Macaca fascicularis).
+474
EKYWCKWSNTGCQTLPSQDEGPSEAFVNCDENSRLVSLTL
NPVTRADEGWYWCGVKQGHFYGETAAVYVAVEEKKVAGS
RYVSPAKADAAPDEKVLDSGVREIENKAIQDPRLFAEEKVVA
DTGDQAGGSRASVDSSSSEEQGGSSKALVSTLVPLGLVLAL
GAVVVGVARARHRKNVDRVSIRSYRTDISMSDFENSREFGA
NDNMGASSITQETSLGGKDEFVATPESTTETKEPKKAKRS
                                                                            +717
```

(The primary amino acid designation refers to the corresponding human amino acid designation as described by Mostov and Kaetzel, Mucosal Immunology. Academic Press, pp. 181-211, 1999.

1. A method of identifying small molecules that specifically bind a transcytotic molecule, comprising contacting a diverse collection of small molecules with at least one transcytotic molecule under conditions where complexes comprising a transcytotic molecule and a small molecule can form, and identifying the small molecules present in said complexes.

2. A method of identifying biologically active small molecules that specifically bind a transcytotic molecule, comprising contacting a diverse collection of small molecules with at least one transcytotic molecule under conditions where complexes comprising a transcytotic molecule and a small molecule can form, and identifying biologically active small molecules present therein.

3. The method of claim 1 or 2, wherein said contacting a diverse collection of small molecules with at least one transcytotic molecule under conditions where complexes comprising a transcytotic molecule and a small molecule can form is repeated at least once.

4. A method of identifying small molecules that specifically bind a pIgR target molecule, comprising contacting a diverse collection of small molecules with at least one pIgR target molecule under conditions where complexes comprising said pIgR target molecule and a small molecule can form, and identifying small molecules present in said complexes.

5. A method of identifying biologically active small molecules that specifically bind a pIgR target molecule, comprising contacting a diverse collection of small molecules with a pIgR target molecule under conditions where complexes comprising said pIgR target molecule and a small molecule can form, and identifying biologically active small molecules present in said complexes.

6. The method of claim 4 or 5, wherein said contacting a diverse collection of small molecules with a pIgR target molecule under conditions where complexes comprising a pIgR target molecule and a small molecule can form is repeated at least once.

7. The method of any one of claims 1, 2, 4 and 5 wherein said collection is a library.

8. The method of any one of claims 1, 2, 4 and 5, wherein said small molecule is capable of undergoing apical endocytosis, apical to basolateral transcytosis, basolateral exocytosis and, additionally or alternatively, apical to basolateral transcytosis.

9. The method of any one of claims 1, 2, 4 and 5 wherein said small molecule is capable of undergoing apical endocytosis.

10. The method of any one of claims 1, 2, 4 and 5 wherein said small molecule is capable of undergoing apical to basolateral transcytosis.

11. The method of claim 10 wherein said small molecule is capable of being delivered to an intercellular location.

12. The method of any one of claims 1, 2, 4 and 5 further comprising separating said complexes from unbound small molecules prior to said identifying.

13. The method of any one of claims 1, 2, 4 and 5 wherein said small molecule is selected from the group consisting of a peptidomimetic and an organic compound.

14. A method of identifying nucleic acids that specifically bind a transcytotic molecule, comprising contacting a diverse collection of nucleic acids with at least one transcytotic molecule under conditions where complexes comprising a transcytotic molecule and a nucleic acid molecule can form, and identifying the nucleic acids present in said complexes.

15. A method of identifying biologically active nucleic acids that specifically bind a transcytotic molecule, comprising contacting a diverse collection of nucleic acids with at least one transcytotic molecule under conditions where complexes comprising a transcytotic molecule and a nucleic acid can form, and identifying nucleic acids present therein that are biologically active.

16. The method of claim 14 or 15, wherein said contacting a diverse collection of nucleic acids with at least one transcytotic molecule under conditions where complexes comprising a transcytotic molecule and a nucleic acid can form is repeated at least once.

17. A method of identifying nucleic acids that specifically bind,a pIgR target molecule, comprising contacting a diverse collection of nucleic acids with at least one pIgR target molecule under conditions where complexes comprising said pIgR target molecule and a nucleic acid can form, and identifying the nucleic acids present in said complexes.

18. A method of identifying biologically active nucleic acids that specifically bind a pIgR target, comprising contacting a diverse collection of nucleic acids with at least one a pIgR target under conditions where complexes comprising a pIgR target and a nucleic acid can form, and identifying biologically active nucleic acids present in said complexes.

19. The method of claim 17 or 18 further comprising separating said complexes from unbound nucleic acids prior to said identifying.

20. The method of claim 17 or 18, wherein said contacting a diverse collection of nucleic acids with a pIgR target molecule under conditions where complexes comprising a pIgR target molecule and a nucleic acid can form is repeated at least once.

21. The method of any one of claims 14, 15, 17 and 18 wherein said collection is a library.

22. The method of any one of claims 14, 15, 17 and 18, wherein said nucleic acid is capable of undergoing apical endocytosis, apical to basolateral transcytosis, basolateral exocytosis and, additionally or alternatively, apical to basolateral transcytosis.

23. The method of any one of claims 14, 15, 17 and 18 wherein said nucleic acid is capable of undergoing apical endocytosis.

24. The method of any one of claims 14, 15, 17 and 18 wherein said nucleic acid is capable of undergoing apical to basolateral transcytosis.

25. The method of claim 24 wherein said nucleic acid is capable of being delivered to an intercellular location.

26. The method of any one of claims 14, 15, 17 and 18 further comprising separating said complexes from unbound nucleic acids prior to said identifying said nucleic acids.

27. The method of any one of claims 14, 15, 17 and 18 wherein said nucleic acid is an aptamer.

28. The method of any one of claims 4, 5, 17 and 18 wherein said pIgR target molecule is a pIgR stalk molecule.

29. The method of any one of claims 4, 5, 17 and 18 wherein said pIgR target molecule is a defined region of pIgR selected from the group consisting of:
R1 From KRSSK (SEQ ID NO: 11) to the carboxy terminus,
R2a From SYRTD (SEQ ID NO: 10) to the carboxy terminus,
R2b From SYRTD (SEQ ID NO: 10) to KRSSK (SEQ ID NO: 11),
R3a From STLVPL (SEQ ID NO: 11) to the carboxy terminus,
R3b From STLVPL (SEQ ID NO: 9) to KRSSK (SEQ ID NO: 11),
R3c From STLVPL (SEQ ID NO: 9) to SYRTD (SEQ ID NO: 10),
R4a From GWYWC (SEQ ID NO: 8) to the carboxy terminus,
R4b From GWYWC (SEQ ID NO: 8) to KRSSK (SEQ ID NO: 11),
R4c From GWYWC (SEQ ID NO: 8) to SYRTD (SEQ ID NO: 10),
R4d From GWYWC (SEQ ID NO: 8) to STLVPL (SEQ ID NO: 9),
R5a From YWCKW (SEQ ID NO: 7) to the carboxy terminus,
R5b From YWCKW (SEQ ID NO: 7) to KRSSK (SEQ ID NO: 11),
R5c From YWCKW (SEQ ID NO: 7) to SYRTD (SEQ ID NO: 10),
R5d From YWCKW (SEQ ID NO: 7) to STLVPL (SEQ ID NO: 9),
R5e From YWCKW (SEQ ID NO: 7) to GWYWC (SEQ ID NO: 8),
R6a From LNQLT (SEQ ID NO: 6) to the carboxy terminus,
R6b From LNQLT (SEQ ID NO: 6) to KRSSK (SEQ ID NO: 11),
R6c From LNQLT (SEQ ID NO: 6) to SYRTD (SEQ ID NO: 10),
R6d From LNQLT (SEQ ID NO: 6) to STLVPL (SEQ ID NO: 9),
R6e From LNQLT (SEQ ID NO: 6) to GWYWC (SEQ ID NO: 8),
R6f From LNQLT (SEQ ID NO: 6) to YWCKW (SEQ ID NO: 7),
R7a From QLFVNEE (SEQ ID NO: 5) to the carboxy terminus,
R7b From QLFVNEE (SEQ ID NO: 5) to KRSSK (SEQ ID NO: 11),
R7c From QLFVNEE (SEQ ID NO: 5) to SYRTD (SEQ ID NO: 10),
R7d From LNQLT (SEQ ID NO: 6) to STLVPL (SEQ ID NO: 9),
R7e From QLFVNEE (SEQ ID NO: 5) to GWYWC (SEQ ID NO: 8),
R7f From QLFVNEE (SEQ ID NO: 5) to YWCKW (SEQ ID NO: 7),
R7g From QLFVNEE (SEQ ID NO: 5) to LNQLT (SEQ ID NO: 6),
R8a From LRKED (SEQ ID NO: 4) to the carboxy terminus,
R8b From LRKED (SEQ ID NO: 4) to KRSSK (SEQ ID NO: 11),
R8c From LRKED (SEQ ID NO: 4) to SYRTD (SEQ ID NO: 10),
R8d From LRKED (SEQ ID NO: 4) to STLVPL (SEQ ID NO: 9),
R8e From LRKED (SEQ ID NO: 4) to GWYWC (SEQ ID NO: 8),
R8f From LRKED (SEQ ID NO: 4) to YWCKW (SEQ ID NO: 7),
R8g From LRKED (SEQ ID NO: 4) to LNQLT (SEQ ID NO: 6), and
R8h From LRKED (SEQ ID NO: 4) to QLFVNEE (SEQ ID NO: 5).

30. The method of any one of claims 4, 5, 17 and 18 wherein said pIgR target molecule is a polypeptide having an amino acid sequence that is conserved among homologs of pIgR.

31. The method of claim 30 wherein said amino acid sequence is selected from the group consisting of LRKED (SEQ ID NO: 4), QLFVNEE (SEQ ID NO: 5, LNQLT (SEQ ID NO: 6), YWCKW (SEQ ID NO: 7), GWYWC (SEQ ID NO: 8), SYRTD (SEQ ID NO: 10) and KRSSK (SEQ ID NO: 11).

32. The method of any one of claims 4, 5, 17 and 18 wherein said pIgR is a pIgR from a vertebrate.

33. The method of claim 32 wherein said vertebrate is a mammal.

34. The method of claim 33 said mammal is selected from the group consisting of human pIgR, simian pIgR, bovine pIgR, murine pIgR, and a possum pIgR.

35. A method of screening for a phage displaying a polypeptide that gives said phage the ability to penetrate a layer of epithelial cells from the apical side of said cells, comprising contacting a diverse collection of phage to the apical side of an epithelial cell layer, and recovering phage on the basolateral side of said layer.

36. A method of screening for a phage displaying a polypeptide that gives said phage paracellular transporting properties, comprising contacting a diverse collection of phage to the apical side of an epithelial cell layer, and recovering phage on the basolateral side of said layer.

37. A method of screening for a phage displaying a polypeptide that gives said phage transcytotic properties, comprising contacting a diverse collection of phage to the apical side of an epithelial cell layer, and recovering phage on the basolateral side of said layer.

38. The method of any one of claims 35, 36 and 37, wherein said epithelial cell line is the MDCK cell line.

43. The method of any one of claims 35, 36 and 37 wherein said cell expresses an exogenous homolog of pIgR.

44. The method of claim 43 wherein said pIgR is from a vertebrate.

45. The method of claim 44 wherein said pIgR is from a mammal.

46. The method of claim 45 said mammal is selected from the group consisting of simian pIgR, bovine pIgR, murine pIgR, and possum pIgR.

47. The method of any one of claims 35, 36 and 37, wherein said apical side is in contact with a first medium that has a different composition than that of a second medium in contact with said basolateral side.

48. The method of claim 49, wherein said second fluid is selected from the group consisting of serum and blood.

49. A method of generating a collection of phage that comprise a focused library of polypeptides that gives said phage the ability to penetrate a layer of epithelial cells from the apical side of said cells, comprising mutagenizing one or more phage identified according to the method of any one of claims 35, 36 and 37.

50. A method of identifying a cellular molecule that causes, enhances or mediates the movement of molecules through epithelial cell barriers.
